(19) United States
(12) Patent Application Publication
(10) Pub. No.: US 2009/0069370 A1
(43) Pub. Date: Mar. 12, 2009

(54) AZACYCLYLISOQUINOLINONE AND ISOINDOLINONE DERIVATIVES AS HISTAMINE-3 ANTAGONISTS

(75) Inventors: Dahui Zhou, East Brunswick, NJ (US); Jonathan Laird Gross, Cranbury, NJ (US); Albert Jean Robichaud, Ringoes, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/208,780

(22) Filed: Sep. 11, 2008

Related U.S. Application Data
(60) Provisional application No. 60/993,554, filed on Sep. 12, 2007.

(51) Int. Cl. A61K 31/4725; C07D 401/04; A61K 31/454; A61P 25/28; C07D 401/10

(52) U.S. Cl. 514/308; 546/141; 514/309; 514/318; 546/200; 546/140

(57) ABSTRACT

The present invention provides a compound of formula I and the use thereof for the treatment of a central nervous system disorder related to or affected by the histamine-3 receptor.

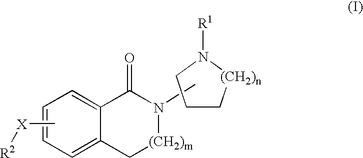

### CROSS-REFERENCE TO RELATED APPLICATIONS

This application claims the benefit under 35 U.S.C. §119(e) to co-pending U.S. provisional application No. 60/993,554, filed Sep. 12, 2007, which is hereby incorporated by reference in its entirety.

### FIELD OF THE INVENTION

The current invention relates to azacyclylisoquinolinone and -isoindolinone compounds, their use in modulation of the histamine-3 (H₃) receptor and treatment of a variety of central nervous system disorders related to or affected by the H₃ receptor. The invention also provides methods of synthesis and pharmaceutical compositions comprising the aminoalkylazole compounds.

### BACKGROUND OF THE INVENTION

The histamine-3 (H₃) receptor is one of four histamine receptor subtypes (H₁-H₄), all of which are members of the G-protein-coupled receptor (GPCR) superfamily. The H₃ receptor is predominantly expressed in the central nervous system. In the brain, it is located in regions associated with learning and memory such as the cerebral cortex, hippocampus and striatum.

The H₃ receptor acts as both an auto- and hetero-receptor to regulate the release of histamine and other neurotransmitters. Within the cortex, the H₃ receptor appears to directly modify GABA release from cortical interneurons. Antagonism of the H₃ receptor produces a decrease in GABA release and disinhibition of the cortical cholinergic system, resulting in increased acetylcholine levels (Bacciottini, L. et al, Behavioral Brain Research, 124, 2001, 183-194). In addition to direct regulation of cholinergic neurotransmission, the H₃ receptor has been shown to modulate the release of dopamine, serotonin and norepinephrine (Leurs, R., et al, Trends in Pharmacological Sciences, 19, 1998, 177-183). Thus, H₃ receptor blockade is able to elevate concentrations of a number of neurotransmitters, including: histamine, acetylcholine, dopamine, serotonin, norepinephrine, and glutamate, and thus offers a means for targeting cognitive processes, which often rely on the integration of multiple neurotransmitter systems.

H₃ agonists have been reported to impair memory in various tasks, such as object recognition, passive avoidance (Blandina, P., et al, British Journal of Pharmacology, 119(8), 1996, 1656-1664) and social olfactory memory (Prast, H., et al, 734, 1996, 316-318), whereas H₃ antagonists have been reported to rescue impairments produced pharmacologically or genetically. Miyazaki, S., et al, Life Sciences, 61, 1997, 355-361; Meguro, K., et al, Pharmacology, Biochemistry and Behavior, 50, 1995, 321-325; Fox, G. B., et. al, Behavioral Brain Research, 131, 2002, 151-161; and Komater, V. A., et al, Psychopharmacology, 167, 2003, 363-372.

H₃ receptors are targets for the control of arousal and vigilance as well as for the treatment of sleep disorders because they colocalize with histaminergic neurons in brain regions that regulate the sleep-wake cycle and they modulate histamine release and levels in the CNS. Passani et al. Trends Pharmacol. Sci. 25, 618-25, 2004. The administration of selective H₃ receptor agonists, such as R-α-methylhistamine, increases sleep time and slow wave sleep in cats and rodents and produces sedation in the guinea pig, whereas H₃ antagonists such as thioperamide increase wakefulness in cats and rats and decrease slow wave sleep and REM sleep in rats. Monti et al. Eur. J. Pharmacol. 205, 283-287, 1991 and Esbenshade et al. Molecular Interventions 6:77-88, 2006.

Studies on memory consolidation and spatial memory impairments, which are particularly prevalent in AD and dimentia, have revealed that the H₃ antagonist thioperamide improves recall in a mouse model of premature senescence as well as in spontaneously hypertensive rat pups, and also prevents scopolamine-induced amnesia. Meguro et al. Pharmacol. Biochem. Behav. 50, 321-325, 1995 and Hancock et al. Expert Opin. Investig. Drugs 13, 1237-1248, 2004. Further, H₃ receptor knockout mice are insensitive to the effects of scopolamine in an inhibitory avoidance paradigm, supporting a role for H₃ receptor modulation of cholinergic function in memory acquisition. Toyota et al. Mol. Pharmacol. 62, 389-397, 2002.

Impairments in social recognition memory are apparent in AD, but may also be relevant to social cognitive impairment in schizophrenia and ADHD. Esbenshade et al. Molecular Interventions 6:77-88, 2006. Social recognition tests have been used to show that the administration of selective histaminergic agonists enhances social memory, whereas recall is disrupted by the inhibition of histamine synthesis. Prast et al. Brain Res. 734, 316-318, 1996. In particular, thioperamide as well as several other H₃ receptor antagonists have been attributed with pro-cognitive effects. Id. In working memory impairments, prevalent in AD, ADHD, and schizophrenia, thioperamide reverses scopolamine-induced deficits. Barbier et al. Br. J. Pharmacol. 143, 649-661, 2004 and Fox et al. J. Pharmacol. Exp. Ther. 305, 897-908, 2003. Thioperamide, ciproxifan, and GT-2331, all H₃ antagonists, are also efficacious in treating impulsivity associated with ADHD in spontaneous hypertensive rat pups. Fox et al. Behav. Brain Res. 131, 151-161, 2002.

The H₃ receptor is also involved in pathological processes in the 6-OHDA (6-hydroxydopamine) lesioned rat brain, a well-characterized model of Parkinson's disease. Increased H₃ receptor mRNA expression and binding may, for example, modulate GABAergic neuronal activity in dopamine-depleted striatum. Anichtchik et al., European Journal of Neuroscience, 12 (11), 3823-3832 2000.

Methamphetamine-induced hyperlocomotor activity, a behaviorally relevant model for psychosis, can be attenuated by ciproxifan in mice (Morisset et al. J. Pharmacol. Exp. Ther. 300, 621-628, 2002), as well as by the antipsychotic drug risperidone and the H₃ receptor antagonist ABT-239. Fox et al. J. Pharmacol. Exp. Ther. 313, 176-190 (2005). H₃ antagonists, such as thioperamide, have also been shown to reduce cumulative food consumption, weight gain and are suggested to have antidepressant activity. Esbenshade et al. supra and Perez-Garcia et al. Psychopharmacologia, 142(2) 215-220. 1999.

Accordingly, there is significant neuroanatomical, neurochemical, pharmacological and behavioral data to support the use of H₃ receptor antagonists for improving cognitive performance in disease states such as neurodegeneration, cognitive impairment, Alzheimer's disease, Parkinson's disease, dementia, psychosis, depression, attention deficit disorder (ADD)/attention deficit hyperactivity disorder (ADHD), schizophrenia, obesity and sleep disorders.

Accordingly, compounds which are inhibitors of the H₃ receptor find use as potential therapeutic agents in the treatment of a variety of central nervous system disorders related to or affected by the H₃ receptor.

### SUMMARY OF THE INVENTION

The present invention provides an azacyclylisoquinolinone or -isoindolinone compound of formula I

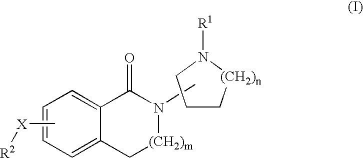

wherein
X is (CR³R⁴)ₚ, CO or O;
m is 0, 1 or 2;
n is 0, 1, 2 or 3;
p is 0, 1 or 2;
R¹ is an alkyl or cycloalkyl group each group optionally substituted;
R² is NR⁵R⁶ or an alkyl, cycloalkyl, cycloheteroalkyl, aryl or heteroaryl group each group optionally substituted with the proviso that when X is O then R² must be other than NR⁵R⁶;
R³ and R⁴ are each independently H, halogen or an optionally substituted alkyl or cycloalkyl group; and
R⁵ and R⁶ each independently H or an alkyl, alkenyl, alkoxy, cycloalkyl, cycloheteroalkyl, aryl or heteroaryl group each group optionally substituted or R⁵ and R⁶ may be taken together with the atom to which they are attached to form an optionally substituted 4- to 7-membered ring optionally containing one or two additional heteroatoms selected from N, O or S or an optionally substituted fused bicyclic or tricyclic 9- to 15-membered aromatic ring system optionally containing one to three additional heteroatoms selected from N, O or S; or
   a stereoisomer thereof or a pharmaceutically acceptable salt thereof;

provided that R¹ is not diphenylpropyl.

In an alternative embodiment of the compound of formula I, R¹ is H.

The present invention also provides methods and compositions useful for the therapeutic treatment of central nervous system disorders related to or affected by the Histamine-3 receptor.

Another embodiment of the present invention provides use of a composition of any one of the embodiments described herein for the treatment of a central nervous system disorder related to or affected by the H₃ receptor. More particularly, the present invention provides for use of a compound of any one of the embodiments described herein for the manufacture of a medicament for the treatment of a central nervous system disorder related to or affected by the H₃ receptor.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

Alzheimer's disease (AD) is characterized by a progressive loss of memory and cognitive function and is the most common cause of dementia in the elderly. AD is believed to affect approximately 15-20 million people worldwide. The goal of treatment in AD, in addition to reversing the disease process, is to improve or at least slow the loss of memory and cognition and to maintain independent function in patients with mild to moderate disease. AD is characterized by numerous deficits in neurotransmitter function (Möller, H-J., European Neuropsychopharmacology, 9, 1999, S53-S59), further a postmortem study in humans suggests that a decrease in brain histamine levels may contribute to the cognitive decline associated with AD, directly or through the cholinergic system (Panula, P., et al, Neuroscience, 82, 1998, 993-997). Histamine-3 (H₃) receptor antagonists have been reported to rescue impairments produced pharmacologically or genetically (Miyazaki, S., et al, Life Sciences, 61, 1997, 355-361; Meguro, K., et al, Pharmacology, Biochemistry and Behavior, 50, 1995, 321-325; Fox, G. B., et. al, Behavioral Brain Research, 131, 2002, 151-161; and Komater, V. A., et al, Psychopharmacology, 167, 2003, 363-372). Neuroanatomical, neurochemical, pharmacological and behavioral data support the belief that H₃ receptor antagonists may improve cognitive performance in disease states such as mild cognitive impairment and Alzheimer's disease and may have therapeutic value in the treatment of attention deficit disorder (ADD)/attention deficit hyperactivity disorder (ADHD), schizophrenia, particularly cognitive dysfunction in schizophrenia, dementia, psychosis, depression, Parkinson's disease, obesity, eating disorders, sleep disorders and neuropathic pain. To that end, compounds which inhibit the H₃ receptor and act as H₃ antagonists are earnestly sought.

Surprisingly it has now been found that pyrrolidinylalkylisoquinolinone and pyrrolidinylalkylisoindolinone compounds of formula I demonstrate H-3 affinity along with significant sub-type selectivity and function as H₃ antagonists. Advantageously, said formula I compounds are effective therapeutic agents for the treatment of central nervous system (CNS) disorders associated with or affected by the H-3 receptor. Accordingly, the present invention provides an azacyclylisoquinolinone or -isoindolinone compound of formula I

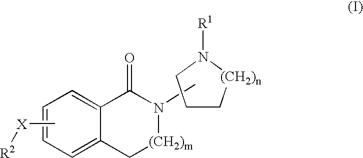

wherein
X is (CR³R⁴)ₚ, CO or O;
m is 0, 1 or 2;
n is 0, 1, 2 or 3;
p is 0, 1 or 2;
R¹ is an alkyl or cycloalkyl group each group optionally substituted;
R² is NR⁵R⁶ or an alkyl, cycloalkyl, cycloheteroalkyl, aryl or heteroaryl group each group optionally substituted with the proviso that when X is O then R₂ must be other than NR⁵R⁶;
R³ and R⁴ are each independently H, halogen or an optionally substituted alkyl or cycloalkyl group; and
R⁵ and R⁶ each independently H or an alkyl, alkenyl, alkoxy, cycloalkyl, cycloheteroalkyl, aryl or heteroaryl group each group optionally substituted or R⁵ and R⁶ may be taken together with the atom to which they are attached to form an optionally substituted 4- to 7-membered ring optionally containing one or two additional heteroatoms selected from N, O or S or an optionally substituted fused bicyclic or tricyclic 9- to 15-membered aromatic ring system optionally containing one to three additional heteroatoms selected from N, O or S; or
   a stereoisomer thereof or a pharmaceutically acceptable salt thereof.
   More particularly, R¹ is not diphenylpropyl.

It is understood that the claims encompass all possible stereoisomers and prodrugs.

Another aspect of the invention provides a method for the treatment of a cognitive disorder related to or affected by the Histamine-3 (H₃) receptor in a patient in need thereof which comprises providing to said patient a therapeutically effective amount of a compound of formula I or any other embodiment thereof described herein. In a more particular embodiment, said disorder is a neurodegenerative disorder. More particular still, said disorder is mild cognitive impairment (MCI), dementia, delirium, amnestic disorder, Alzheimer's disease (AD), Parkinson's disease (PD), Huntington's disease (HD), memory disorder, memory deficits associated with depression, schizophrenia, a psychotic disorder, paranoia, mano-depressive illness, attention deficit disorder (ADD), attention deficit hyperactivity disorder (ADHD), dyslexia, developmental disorders, Down's syndrome, Fragile X syndrome, loss of executive function, loss of learned information, vascular dementia, cognitive decline, neurodegenerative disorder, HIV-induced dimentia, head trauma, Pick's disease, Creutzfeldt-Jakob disease, Body dementia, vascular dementia, surgical procedure-induced cognitive dysfunction, traumatic brain injury or stroke. In another more particular embodiment, said disorder is selected from the group consisting of: Alzheimer's disease, attention deficit disorder, schizophrenia; Parkinsons' disease, frontal temporal dementia or depression.

Another aspect of the invention provides a method for the inhibition of an H₃ receptor comprising contacting said receptor with an effective amount of a compound of formula I or any other embodiment thereof described herein.

An additional aspect of the invention provides a pharmaceutical composition which comprises a pharmaceutically acceptable carrier and an effective amount of a compound of formula I or any other embodiment thereof described herein.

“Treating” or “treatment” of a disease in a subject refers to inhibiting the disease or arresting its development; ameliorating symptoms of the disease; or causing regression of the disease.

Additionally, the compound of the invention may be used in the prevention of a disease described herein.

A “cognitive disease,” “cognitive dysfunction,” or “cognition-related disorder” is a disease or disorder affecting mental processes such as memory, attention, perception, action, problem solving and mental imagery. Cognitive dysfunction generally originates in the central nervous system and can be influenced or derived from neurodegeneration. Particular cognition-related disorders (e.g., cognitive dysfunction) include, without limitation, mild cognitive impairment (MCI), dementia, delirium, amnestic disorder, Alzheimer's disease, Parkinson's disease, Huntington's disease, memory disorders including memory deficits associated with depression, senile dementia, dementia of Alzheimer's disease, cognitive deficits or cognitive dysfunction associated with neurological conditions including, for example, Parkinson's disease (PD), Huntington's disease (HD), Alzheimer's disease, depression and schizophrenia (and other psychotic disorders such as paranoia and mano-depressive illness); cognitive dysfunction in schizophrenia, disorders of attention and learning such as attention deficit disorder (ADD), attention deficit hyperactivity disorder (ADHD), and dyslexia, cognitive dysfunction associated with developmental disorders such as Down's syndrome and Fragile X syndrome, loss of executive function, loss of learned information, vascular dementia, schizophrenia, cognitive decline, neurodegenerative disorder, and other dementias, for example, due to HIV disease, head trauma, Parkinson's disease, Huntington's disease, Pick's disease, Creutzfeldt-Jakob disease, or due to multiple etiologies. Cognition-related disorders also include, without limitation, cognitive dysfunction associated with MCI and dementias such as Lewy Body, vascular, and post stroke dementias. Cognitive dysfunction associated with surgical procedures, traumatic brain injury or stroke may also be treated in accordance with the embodiments described herein.

The term “H₃ antagonist” or “H₃ inhibitor” as used herein refers to a composition that reduces activity of the H₃ receptor. H₃ antagonists described herein can either reduce constitutive H₃ activity independent of agonist interaction (i.e. function as an inverse agonist) or reduce H₃ agonist-mediated activity.

An optionally substituted moiety may be substituted with one or more substituents, which may be the same or different. The substituent groups, which are optionally present, may be one or more of those customarily employed in the development of pharmaceutical compounds or the modification of such compounds to influence their structure/activity, persistence, absorption, stability or other beneficial property. Specific examples of such substituents include halogen atoms, nitro, cyano, thiocyanato, cyanato, hydroxyl, alkyl, haloalkyl, alkoxy, haloalkoxy, amino, alkylamino, dialkylamino, formyl, alkoxycarbonyl, carboxyl, alkanoyl, alkylthio, alkylsuphinyl, alkylsulphonyl, carbamoyl, alkylamido, phenyl, phenoxy, benzyl, benzyloxy, heterocyclyl or cycloalkyl groups, preferably halogen atoms or lower alkyl or lower alkoxy groups. Unless otherwise specified, typically, 0 to 4, 0 to 3, 0 to 2 or 0 to 1 substituents may be present. Optionally substituted groups may themselves be substituted with up to three levels of substitution.

Preferably, optionally substituted refers to the replacement of 0 to 4, 0 to 3, 0 to 2 or 0 to 1 hydrogen atoms with 0 to 4, 0 to 3, 0 to 2 or 0 to 1 groups selected from C₁-C₆ alkyl, C₃-C₆ cycloakyl, C₂-C₆ alkenyl, C₂-C₆ alkynyl, halo, nitro, cyano, hydroxy, C₆-C₁₀ aryl, a 3-10 membered heterocyclyl ring, a 5-10 membered heteroaryl ring, —N(R^{a})₂, —C(O)R^{b}, —OR^{c} and —S(O)ₚR^{d}; wherein each R^{a} is independently H, C₁-C₄ alkyl, —CHO, —C(O)(C₁-C₄ alkyl), or —CO₂(C₁-C₄ alkyl); each R^{b} is independently H, —OH, —O(C₁-C₄), C₁-C₄ alkyl, —NH₂, —NH(C₁-C₄ alkyl), or —N(C₁-C₄ alkyl)₂; each R^{c} is independently H, C₁-C₄ alkyl optionally substituted with halo, —CHO or —C(O)(C₁-C₄ alkyl); each R^{d} is independently C₁-C₄ alkyl, or —OH; and p is 0, 1 or 2. A suitable group of substituents is CN, OH, —NH₂, —NH(C₁-C₄ alkyl), or —N(C₁-C₄ alkyl)₂; halogen, phenyl, carbamoyl, carbonyl, alkoxy or aryloxy.

As used herein, the term alkyl refers to a linear or branched alkyl moiety containing up to 12 carbon atoms, e.g. up to 10 carbon atoms, preferably up to 6 carbon atoms, more preferably up to 4 carbon atoms. Examples of saturated hydrocarbon alkyl moieties include, but are not limited to, chemical groups such as methyl, ethyl, n-propyl, isopropyl, n-butyl, tert-butyl, isobutyl, sec-butyl; higher homologs such as n-pentyl, n-hexyl, and the like.

As used herein, the term haloalkyl designates a CₙH₂ₙ₊₁ group having from one to 2n+1 halogen atoms which may be the same or different. Examples of haloalkyl groups include CF₃, CH₂Cl, C₂H₃BrCl, C₃H₅F₂, or the like.

The term halogen, as used herein, designates fluorine, chlorine, bromine, and iodine.

The term alkenyl, as used herein, refers to either a (C₂-C₁₀) straight chain or (C₃-C₁₀) branched-chain monovalent hydrocarbon moiety containing at least one double bond. The alkenyl is suitably a (C₂-C₈), (C₂-C₆), (C₂-C₄) or (C₂-C₃) moiety. Such hydrocarbon alkenyl moieties may be mono or polyunsaturated, and may exist in the E or Z configurations. The compounds of this invention are meant to include all possible E and Z configurations. Examples of mono or polyunsaturated hydrocarbon alkenyl moieties include, but are not limited to, chemical groups such as vinyl, 2-propenyl, isopropenyl, crotyl, 2-isopentenyl, butadienyl, 2-(butadienyl), 2,4-pentadienyl, 3-(1,4-pentadienyl), and higher homologs, isomers, or the like.

The term alkynyl, as used in the specification and claims, designates either a (C₂-C₁₀) straight chain or (C₃-C₁₀) branched chain monovalent hydrocarbon moiety having at least one triple bond. The alkynyl is suitably a (C₂-C₈), (C₂-C₆), (C₂-C₄) or (C₂-C₃) moiety. Such hydrocarbon alkynyl moieties may be mono or polyunsaturated, and may exist in the E or Z configurations. The compounds of this invention are meant to include all possible E and Z configurations. Examples of mono or polyunsaturated hydrocarbon alkynyl moieties include, but are not limited to, propynyl, butynyl, 1,3-butadiynyl, pentynyl, hexynyl, or the like.

The term cycloalkyl, as used herein, refers to a monocyclic, bicyclic, tricyclic, fused, bridged, or spiro monovalent saturated hydrocarbon moiety of 3-10 carbon atoms. The cycloalkyl is suitably a (C₃-C₈) or a (C₃-C₆) moiety. Examples of cycloalkyl moieties include, but are not limited to, chemical groups such as cyclopropyl, cyclobutyl, cyclopentyl, cyclohexyl, cycloheptyl, norbornyl, adamantyl, spiro[4.5]decanyl, or the like.

The term cycloheteroalkyl, as used herein, designates one or more (fused if more than one) 5-7 membered ring systems containing 1, 2 or 3 heteroatoms, which may be the same or different, selected from N, O or S and optionally containing at least one double bond. Exemplary of the cycloheteroalkyl ring systems included in the term as designated herein are the following rings wherein X₁ is NR′, O or S and R′ is H or an optional substituent as defined hereinabove (when there are two X₁ groups they may be the same or different).

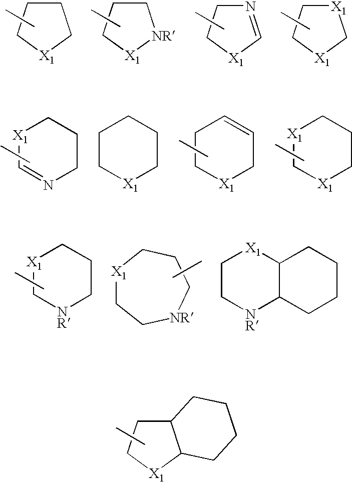

The term aryl, as used herein, refers to an aromatic carbocyclic moiety of up to 20 carbon atoms, which may be a single ring (monocyclic) or multiple rings (up to three rings) fused together. Examples of aryl moieties include, but are not limited to, chemical groups such as phenyl, 1-naphthyl, 2-naphthyl, anthryl, or the like. Aryl also includes polycyclic rings containing heterocyclic rings that are appended through the aromatic carbocyclic ring (e.g. 1,3-benzodioxol-5-yl).

The term heteroaryl as used herein designates an aromatic heterocyclic ring system, which may be a single ring (monocyclic) or multiple rings (up to three rings) fused together. The rings may contain from one to four hetero atoms selected from nitrogen, oxygen, or sulfur, which may be the same or different, wherein the nitrogen or sulfur atoms are optionally oxidized, or the nitrogen atom is optionally quarternized. Examples of heteroaryl moieties include, but are not limited to, heterocycles such as furan, thiophene, pyrrole, pyrazole, imidazole, oxazole, isoxazole, thiazole, isothiazole, oxadiazole, triazole, pyridine, pyrimidine, pyrazine, pyridazine, benzimidazole, benzoxazole, benzisoxazole, benzothiazole, benzofuran, benzothiophene, thianthrene, dibenzofuran, dibenzothiophene, indole, indazole, azaindole, azaindazole, quinoline, isoquinoline, quinazoline, quinoxaline, purine, or the like.

As used herein: EDC designates 1-(3-dimethylaminopropyl)-3-ethylcarbo-diimide hydrochloride; HOBt designates 1-hydroxybenzotriazole; DIPEA designates diisopropylethylamine; Burgess Reagent designates (methoxycarbonylsulfamoyl)-triethylammonium hydroxide, inner salt; and DBU designates 1,8-diazabicyclo[5.4.0]-undec-7-ene.

Unless otherwise stated, structures depicted herein are also meant to include all stereochemical forms of the structure; i.e., the R and S configurations for each asymmetric center and geometric isomers around a double bond (E and Z). Therefore, single stereochemical isomers as well as enantiomeric and diastereomeric mixtures of the present compounds are within the scope of the invention. Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C— or ¹⁴C-enriched carbon are within the scope of this invention.

Unless indicated otherwise, the nomenclature of substituents that are not explicitly defined herein are arrived at by naming the terminal portion of the functionality followed by the adjacent functionality toward the point of attachment.

The compounds of the present invention may be converted to salts, in particular pharmaceutically acceptable salts using art recognized procedures. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono-, di- or tri-lower alkylamine, for example ethyl-tert-butyl-, diethyl-, diisopropyl-, triethyl-, tributyl- or dimethylpropylamine, or a mono-, di-, or trihydroxy lower alkylamine, for example mono-, di- or triethanolamine. Internal salts may furthermore be formed. Salts which are unsuitable for pharmaceutical uses but which can be employed, for example, for the isolation or purification of free compounds or their pharmaceutically acceptable salts, are also included. The term “pharmaceutically acceptable salt”, as used herein, refers to salts derived from organic and inorganic acids such as, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids when a compound of this invention contains a basic moiety. Salts may also be formed from organic and inorganic bases, preferably alkali metal salts, for example, sodium, lithium, or potassium, when a compound of this invention contains a carboxylate or phenolic moiety, or similar moiety capable of forming base addition salts.

Compounds of the invention include esters, carbamates or other conventional prodrug forms, which in general, are functional derivatives of the compounds of the invention and which are readily converted to the inventive active moiety in vivo. Correspondingly, the method of the invention embraces the treatment of the various conditions described hereinabove with a compound of formula I or with a compound which is not specifically disclosed but which, upon administration, converts to a compound of formula I in vivo. Also included are metabolites of the compounds of the present invention defined as active species produced upon introduction of these compounds into a biological system.

Preferred compounds of the invention are those compounds of formula I wherein n is 1 or 2. Another group of preferred compounds is those formula I compounds wherein R¹ is an optionally substituted cycloalkyl group. In one embodiment of the invention, preferred compounds of formula I are those compounds having the structure of formula Ia

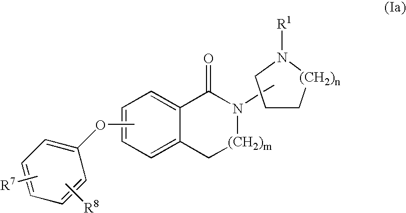

wherein
m is 0, 1 or 2;
n is 0, 1, 2 or 3;
R¹ is an alkyl or cycloalkyl group each group optionally substituted;
R⁷ and R⁸ are each independently H, halogen, CN, CONR⁹R¹⁰, OR¹¹, CO₂R¹¹, COR¹¹, or an alkyl, haloalkyl or cycloalkyl group each group optionally substituted; R⁹ and R¹⁰ are each independently H or an alkyl, haloalkyl, cycloalkyl, aryl or heteroaryl group each group optionally substituted or R⁹ and R¹⁰ may be taken together with the atom to which they are attached to form an optionally substituted 5- to 7-membered ring optionally containing one or two additional heteroatoms selected from N, O or S; and
R¹¹ is H or an alkyl, haloalkyl, cycloalkyl, alkenyl, alkynyl, aryl or heteroaryl group each group optionally substituted; or
   a stereoisomer thereof or a pharmaceutically acceptable salt thereof.

In an alternative embodiment of the compound of formula I, R¹ is H.

In another embodiment of the invention, preferred compounds of formula I wherein X is (CR³R⁴)ₚ and p is 0. Another group of preferred compounds is those formula I compounds wherein R¹ is an optionally substituted cycloalkyl group. In one embodiment of the invention, preferred compounds of formula I are those compounds having the structure of formula Ia formula Ib

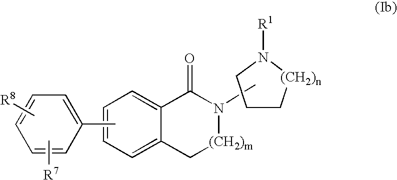

wherein
m is 0, 1 or 2;
n is 0, 1, 2 or 3;
R¹ is an alkyl or cycloalkyl group each group optionally substituted;
R⁷ and R⁸ are each independently H, halogen, CN, CONR⁹R¹⁰, OR¹¹, CO₂R¹¹, COR¹¹, or an alkyl, haloalkyl or cycloalkyl group each group optionally substituted; R⁹ and R¹⁰ are each independently H or an alkyl, haloalkyl, cycloalkyl, aryl or heteroaryl group each group optionally substituted or R⁹ and R¹⁰ may be taken together with the atom to which they are attached to form an optionally substituted 5- to 7-membered ring optionally containing one or two additional heteroatoms selected from N, O or S; and
R¹¹ is H or an alkyl, haloalkyl, cycloalkyl, alkenyl, alkynyl, aryl or heteroaryl group each group optionally substituted; or

a stereoisomer thereof or a pharmaceutically acceptable salt thereof

provided that R¹ is not diphenylpropyl.

More preferred compounds of the invention are those compounds of formula I wherein the azacyclic ring is attached at the 3-position of pyrrolidine, said azacyclic ring. Another group of more preferred compounds is those compounds of formula Ia wherein m is 1 and n is 1 or 2 and R¹ is an optionally substituted cycloalkyl group. A further group of more preferred compounds are those compounds of formula Ia wherein the azacyclic ring is attached at the 3-position of pyrrolidine, said azacyclic ring; R⁷ is CONR⁹R¹⁰; and R⁸ is H or halogen.

In another preferred embodiment, R¹ is C₁-C₄ alkyl. Alternatively, R¹ is a C₃-C₆ cycloalkyl.

In another preferred embodiment, the compound has the structure of formula Ix:

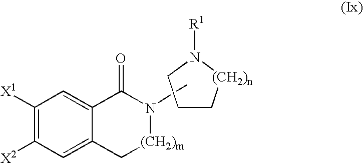

wherein,

X¹ is H and X² is —X—R²; or

X¹ is —X—R² and X² is H; and

the remaining variables are as defined in formula I.

More particularly, X¹ is H and X² is —X—R². Alternatively, X¹ is —X—R² and X² is H.

In another embodiment, R² is an optionally substituted aminocarbonylphenyl group. In another embodiment, R² is an optionally substituted cycloheteroalkylcarbonylphenyl group. In a particular embodiment, when R² is an aminocarbonylphenyl group, the optional substitution at the amino group is alkyl or cycloalkyl and the optional substitution at the phenyl group is halo.

In another embodiment, R² is selected from the group consisting of methyloxycarbonylphenyl, carboxyphenyl, aminocarbonylphenyl, alkylaminocarbonylphenyl, cycloalkylaminocarbonylphenyl, N,N-dialkylaminocarbonylphenyl, carboxyphenylalkyl, aminocarbonylphenylalkyl, alkylaminocarbonylphenylalkyl, N,N-dialkylaminocarbonylphenylalkyl, cycloalkylaminocarbonylphenylalkyl, cyanophenyl, cycloheteroalkylcarbonylphenyl, aminocarbonylhalophenyl, alkylaminocarbonylhalophenyl, N,N-dialkylaminocarbonylhalophenyl, cycloheteroalkylcarbonylhalophenyl, halophenyl, phenyl, dihalophenyl, alkylaminocarbonylhalophenyl, pyrrolidine-1-carbonylphenyl, and aminoalkoxyalkyl.

Among the preferred compounds of the invention are:
4-{[2-(1-Cyclobutylpiperidin-4-yl)-1-oxo-1,2,3,4-tetrahydroisoquinolin-6-yl]oxy}benzoic acid;
Methyl 4-{[2-(1-cyclobutylpiperidin-4-yl)-1-oxo-1,2,3,4-tetrahydroisoquinolin-6-yl]oxy}benzoate;
4-{[2-(1-Cyclobutylpiperidin-4-yl)-1-oxo-1,2,3,4-tetrahydroisoquinolin-6-yl]oxy}benzamide;
4-{[2-(1-Cyclobutylpiperidin-4-yl)-1-oxo-1,2,3,4-tetrahydroisoquinolin-6-yl]oxy}-N-methylbenzamide;
4-{[2-(1-Cyclobutylpiperidin-4-yl)-1-oxo-1,2,3,4-tetrahydroisoquinolin-6-yl]oxy}-N-ethylbenzamide;
4-{[2-(1-Cyclobutylpiperidin-4-yl)-1-oxo-1,2,3,4-tetrahydroisoquinolin-6-yl]oxy}-N-isopropylbenzamide;
4-{[2-(1-Cyclobutylpiperidin-4-yl)-1-oxo-1,2,3,4-tetrahydroisoquinolin-6-yl]oxy}-N-cyclopropylbenzamide;
N-Cyclobutyl-4-{[2-(1-cyclobutylpiperidin-4-yl)-1-oxo-1,2,3,4-tetrahydroisoquinolin-6-yl]oxy}benzamide;
4-{[2-(1-Cyclobutylpiperidin-4-yl)-1-oxo-1,2,3,4-tetrahydroisoquinolin-6-yl]oxy}-N-cyclopentylbenzamide;
4-{[2-(1-Cyclobutylpiperidin-4-yl)-1-oxo-1,2,3,4-tetrahydroisoquinolin-6-yl]oxy}-N,N-dimethylbenzamide;
4-{[2-(1-Cyclobutylpiperidin-4-yl)-1-oxo-1,2,3,4-tetrahydroisoquinolin-6-yl]oxy}-N,N-diethylbenzamide;
2-(1-Cyclobutylpiperidin-4-yl)-6-[4-(pyrrolidin-1-ylcarbonyl)phenoxy]-3,4-dihydroisoquinolin-1(2H)-one;
Methyl 4-({[2-(1-Cyclobutylpiperidin-4-yl)-1-oxo-1,2,3,4-tetrahydroisoquinolin-6-yl]oxy}methyl)benzoate;
4-({[2-(1-Cyclobutylpiperidin-4-yl)-1-oxo-1,2,3,4-tetrahydroisoquinolin-6-yl]oxy}methyl)benzoic acid;
4-({[2-(1-Cyclobutylpiperidin-4-yl)-1-oxo-1,2,3,4-tetrahydroisoquinolin-6-yl]oxy}methyl)benzamide;
4-({[2-(1-Cyclobutylpiperidin-4-yl)-1-oxo-1,2,3,4-tetrahydroisoquinolin-6-yl]oxy}methyl)-N-methylbenzamide;
4-({[2-(1-Cyclobutylpiperidin-4-yl)-1-oxo-1,2,3,4-tetrahydroisoquinolin-6-yl]oxy}methyl)-N-ethylbenzamide
4-({[2-(1-Cyclobutylpiperidin-4-yl)-1-oxo-1,2,3,4-tetrahydroisoquinolin-6-yl]oxy}methyl)-N,N-dimethylbenzamide;
N-Cyclobutyl-4-({[2-(1-cyclobutylpiperidin-4-yl)-1-oxo-1,2,3,4-tetrahydroisoquinolin-6-yl]oxy}methyl)benzamide;
2-(1-Cyclobutylpiperidin-4-yl)-6-{[4-(pyrrolidin-1-ylcarbonyl)benzyl]oxy}-3,4-dihydroisoquinolin-1(2H)-one;
4-({[2-(1-Cyclobutylpiperidin-4-yl)-1-oxo-1,2,3,4-tetrahydroisoquinolin-6-yl]oxy}methyl)-N-cyclopentylbenzamide;
4-(1-Oxo-2-piperidin-4-yl-2,3-dihydro-1H-isoindol-5-yl)benzonitrile;
4-[2-(1-Methylpiperidin-4-yl)-1-oxo-2,3-dihydro-1H-isoindol-5-yl]benzonitrile;
4-[2-(1-Ethylpiperidin-4-yl)-1-oxo-2,3-dihydro-1H-isoindol-5-yl]benzonitrile;
4-[1-Oxo-2-(1-propylpiperidin-4-yl)-2,3-dihydro-1H-isoindol-5-yl]benzonitrile;
4-{2-[1-(Cyclopropylmethyl)piperidin-4-yl]-1-oxo-2,3-dihydro-1H-isoindol-5-yl}benzonitrile;
4-{2-[1-(Cyclopentylmethyl)piperidin-4-yl]-1-oxo-2,3-dihydro-1H-isoindol-5-yl}benzonitrile;
4-[2-(1-Cyclobutylpiperidin-4-yl)-1-oxo-2,3-dihydro-1H-isoindol-5-yl]benzonitrile;
4-[2-(1-Cyclopentylpiperidin-4-yl)-1-oxo-2,3-dihydro-1H-isoindol-5-yl]benzonitrile;
4-[2-(1-Cyclohexylpiperidin-4-yl)-1-oxo-2,3-dihydro-1H-isoindol-5-yl]benzonitrile;
4-({2-[(3R)-1-cyclobutylpyrrolidin-3-yl]-1-oxo-1,2,3,4-tetrahydroisoquinolin-6-yl}oxy)benzoic acid;
4-({2-[(3R)-1-cyclobutylpyrrolidin-3-yl]-1-oxo-1,2,3,4-tetrahydroisoquinolin-6-yl}oxy)-N-methylbenzamide;
4-({2-[(3R)-1-cyclobutylpyrrolidin-3-yl]-1-oxo-1,2,3,4-tetrahydroisoquinolin-6-yl}oxy)-N-ethylbenzamide;
2-[(3R)-1-cyclobutylpyrrolidin-3-yl]-6-[4-(pyrrolidin-1-ylcarbonyl)phenoxy]-3,4-dihydroisoquinolin-1(2H)-one;
4-({2-[(3R)-1-cyclopentylpyrrolidin-3-yl]-1-oxo-1,2,3,4-tetrahydroisoquinolin-6-yl}oxy)benzoic acid;
4-({2-[(3R)-1-cyclopentylpyrrolidin-3-yl]-1-oxo-1,2,3,4-tetrahydroisoquinolin-6-yl}oxy)-N-methylbenzamide;
4-({2-[(3R)-1-cyclopentylpyrrolidin-3-yl]-1-oxo-1,2,3,4-tetrahydroisoquinolin-6-yl}oxy)-N-ethylbenzamide;
2-[(3R)-1-cyclopentylpyrrolidin-3-yl]-6-[4-(pyrrolidin-1-ylcarbonyl)phenoxy]-3,4-dihydroisoquinolin-1(2H)-one;
4-{2-[(3R)-1-cyclopentylpyrrolidin-3-yl]-1-oxo-1,2,3,4-tetrahydroisoquinolin-6-yl}-N-methylbenzamide;
4-{2-[(3R)-1-cyclopentylpyrrolidin-3-yl]-1-oxo-1,2,3,4-tetrahydroisoquinolin-6-yl}-N-ethylbenzamide;
2-[(3R)-1-cyclopentylpyrrolidin-3-yl]-6-[4-(pyrrolidin-1-ylcarbonyl)phenyl]-3,4-dihydroisoquinolin-1(2H)-one;
4-{2-[(3R)-1-cyclobutylpyrrolidin-3-yl]-1-oxo-1,2,3,4-tetrahydroisoquinolin-6-yl}-N-methylbenzamide;
4-{2-[(3R)-1-cyclobutylpyrrolidin-3-yl]-1-oxo-1,2,3,4-tetrahydroisoquinolin-6-yl}-N-ethylbenzamide;
2-[(3R)-1-cyclobutylpyrrolidin-3-yl]-6-[4-(pyrrolidin-1-ylcarbonyl)phenyl]-3,4-dihydroisoquinolin-1(2H)-one;
2-[(3R)-1-(cyclopentylmethyl)pyrrolidin-3-yl]-6-[4-(pyrrolidin-1-ylcarbonyl)phenyl]-3,4-dihydroisoquinolin-1(2H)-one;
2-[(3R)-1-methylpyrrolidin-3-yl]-6-[4-(pyrrolidin-1-ylcarbonyl)phenyl]-3,4-dihydroisoquinolin-1(2H)-one;
2-[(3R)-1-(cyclopropylmethyl)pyrrolidin-3-yl]-6-[4-(pyrrolidin-1-ylcarbonyl)phenyl]-3,4-dihydroisoquinolin-1(2H)-one;
2-[(3R)-1-(furan-3-ylmethyl)pyrrolidin-3-yl]-6-[4-(pyrrolidin-1-ylcarbonyl)phenyl]-3,4-dihydroisoquinolin-1(2H)-one;
2-[(3R)-1-cyclohexylpyrrolidin-3-yl]-6-[4-(pyrrolidin-1-ylcarbonyl)phenyl]-3,4-dihydroisoquinolin-1(2H)-one;
2-[(3R)-1-(furan-2-ylmethyl)pyrrolidin-3-yl]-6-[4-(pyrrolidin-1-ylcarbonyl)phenyl]-3,4-dihydroisoquinolin-1(2H)-one;
6-[4-(pyrrolidin-1-ylcarbonyl)phenyl]-2-[(3R)-1-(thiophen-2-ylmethyl)pyrrolidin-3-yl]-3,4-dihydroisoquinolin-1(2H)-one;
2-[(3R)-1-(1-methylethyl)pyrrolidin-3-yl]-6-[4-(pyrrolidin-1-ylcarbonyl)phenyl]-3,4-dihydroisoquinolin-1(2H)-one;
2-(1-cyclobutylpiperidin-4-yl)-6-[4-(pyrrolidin-1-ylcarbonyl)phenyl]-3,4-dihydroisoquinolin-1(2H)-one;
2-[(3R)-piperidin-3-yl]-6-[4-(pyrrolidin-1-ylcarbonyl)phenyl]-3,4-dihydroisoquinolin-1(2H)-one;
2-(1-cyclopentylpiperidin-4-yl)-6-[4-(pyrrolidin-1-ylcarbonyl)phenyl]-3,4-dihydroisoquinolin-1(2H)-one;
2-[(3R)-1-cyclopentylpiperidin-3-yl]-6-[4-(pyrrolidin-1-ylcarbonyl)phenyl]-3,4-dihydroisoquinolin-1(2H)-one;
2-[(3R)-1-cyclobutylpiperidin-3-yl]-6-[4-(pyrrolidin-1-ylcarbonyl)phenyl]-3,4-dihydroisoquinolin-1(2H)-one;
2-[(3R)-1-cyclobutylpyrrolidin-3-yl]-6-(2-fluoro-4-{[(2S)-2-methylpyrrolidin-1-yl]carbonyl}phenyl)-3,4-dihydroisoquinolin-1(2H)-one;
N-cyclobutyl-4-{2-[(3R)-1-cyclobutylpyrrolidin-3-yl]-1-oxo-1,2,3,4-tetrahydroisoquinolin-6-yl}-3-fluorobenzamide;
4-{2-[(3R)-1-cyclobutylpyrrolidin-3-yl]-1-oxo-1,2,3,4-tetrahydroisoquinolin-6-yl}-3-fluoro-N-methylbenzamide;
2-[(3R)-1-cyclobutylpyrrolidin-3-yl]-6-(2-fluoro-4-{[(2R)-2-methylpyrrolidin-1-yl]carbonyl}phenyl)-3,4-dihydroisoquinolin-1(2H)-one;
4-{2-[(3R)-1-cyclobutylpyrrolidin-3-yl]-1-oxo-1,2,3,4-tetrahydroisoquinolin-6-yl}-N-ethyl-3-fluorobenzamide;
4-{2-[(3R)-1-cyclobutylpyrrolidin-3-yl]-1-oxo-1,2,3,4-tetrahydroisoquinolin-6-yl}-3-fluoro-N,N-dimethylbenzamide;
2-[(3R)-1-cyclobutylpyrrolidin-3-yl]-6-[2-fluoro-4-(pyrrolidin-1-ylcarbonyl)phenyl]-3,4-dihydroisoquinolin-1(2H)-one;
4-{2-[(3R)-1-cyclobutylpyrrolidin-3-yl]-1-oxo-1,2,3,4-tetrahydroisoquinolin-6-yl}-N-cyclopentyl-3-fluorobenzamide;
4-{2-[(3R)-1-cyclobutylpyrrolidin-3-yl]-1-oxo-1,2,3,4-tetrahydroisoquinolin-6-yl}-N-cyclopropyl-3-fluorobenzamide;
2-[(3R)-1-cyclobutylpyrrolidin-3-yl]-6-[4-fluoro-3-(pyrrolidin-1-ylcarbonyl)phenyl]-3,4-dihydroisoquinolin-1(2H)-one;
2-[(3R)-1-cyclobutylpyrrolidin-3-yl]-6-[3-fluoro-4-(pyrrolidin-1-ylcarbonyl)phenyl]-3,4-dihydroisoquinolin-1(2H)-one;
6-[3-chloro-4-(pyrrolidin-1-ylcarbonyl)phenyl]-2-[(3R)-1-cyclobutylpyrrolidin-3-yl]-3,4-dihydroisoquinolin-1(2H)-one;
2-(1-isopropylpiperidin-4-yl)-6-[4-(pyrrolidin-1-ylcarbonyl)phenyl]-3,4-dihydroisoquinolin-1(2H)-one;
2-[(3R)-1-isopropylpiperidin-3-yl]-6-[4-(pyrrolidin-1-ylcarbonyl)phenyl]-3,4-dihydroisoquinolin-1(2H)-one;
2-[(3R)-1-cyclobutylpyrrolidin-3-yl]-6-[3-(pyrrolidin-1-ylcarbonyl)phenyl]-3,4-dihydroisoquinolin-1(2H)-one;
4-{2-[(3R)-1-cyclobutylpyrrolidin-3-yl]-1-oxo-1,2,3,4-tetrahydroisoquinolin-6-yl}-N,N-dimethylbenzamide;
2-[(3R)-1-cyclobutylpyrrolidin-3-yl]-N-cyclopentyl-1-oxo-1,2,3,4-tetrahydroisoquinoline-6-carboxamide;
2-[(3R)-1-cyclobutylpyrrolidin-3-yl]-6-(pyrrolidin-1-ylcarbonyl)-3,4-dihydroisoquinolin-1(2H)-one;
N-ethyl-3-fluoro-4-{2-[(3R)-1-isopropylpyrrolidin-3-yl]-1-oxo-1,2,3,4-tetrahydroisoquinolin-6-yl}benzamide;
3-fluoro-4-{2-[(3R)-1-isopropylpyrrolidin-3-yl]-1-oxo-1,2,3,4-tetrahydroisoquinolin-6-yl}-N-methylbenzamide;
N-ethyl-4-{2-[(3R)-1-isopropylpyrrolidin-3-yl]-1-oxo-1,2,3,4-tetrahydroisoquinolin-6-yl}benzamide;
6-[2-fluoro-4-(pyrrolidin-1-ylcarbonyl)phenyl]-2-[(3R)-1-isopropylpyrrolidin-3-yl]-3,4-dihydroisoquinolin-1(2H)-one;
2-[(3R)-1-cyclobutylpyrrolidin-3-yl]-6-phenyl-3,4-dihydroisoquinolin-1(2H)-one
2-[(3R)-1-cyclobutylpyrrolidin-3-yl]-6-(4-fluorophenyl)-3,4-dihydroisoquinolin-1(2H)-one;
4-{2-[(3R)-1-cyclobutylpyrrolidin-3-yl]-1-oxo-2,3-dihydro-1H-isoindol-5-yl}-N-methylbenzamide;
2-[(3R)-1-cyclobutylpyrrolidin-3-yl]-5-[4-(pyrrolidin-1-ylcarbonyl)phenyl]isoindolin-1-one;
2-[(3R)-1-cyclobutylpyrrolidin-3-yl]-3,3′,4,4′-tetrahydro-6,6′-biisoquinoline-1,1′(2H,2′H)-dione;
2-[(3R)-1-benzylpyrrolidin-3-yl]-6-[4-(pyrrolidin-1-ylcarbonyl)phenyl]-3,4-dihydroisoquinolin-1(2H)-one;
2-[(3R)-pyrrolidin-3-yl]-6-[4-(pyrrolidin-1-ylcarbonyl)phenyl]-3,4-dihydroisoquinolin-1(2H)-one;
4-{2-[(3S)-1-cyclobutylpyrrolidin-3-yl]-1-oxo-1,2,3,4-tetrahydroisoquinolin-6-yl}-N-methylbenzamide;
2-(1-benzylpiperidin-4-yl)-6-[4-(pyrrolidin-1-ylcarbonyl)phenyl]-3,4-dihydroisoquinolin-1(2H)-one;
2-[(3R)-1-benzylpiperidin-3-yl]-6-[4-(pyrrolidin-1-ylcarbonyl)phenyl]-3,4-dihydroisoquinolin-1(2H)-one;
(R)-4-(2-(1-cyclobutylpyrrolidin-3-yl)-1-oxo-1,2,3,4-tetrahydroisoquinolin-6-yl)-N-isopropylbenzamide;
(R)-4-(2-(1-cyclobutylpyrrolidin-3-yl)-1-oxo-1,2,3,4-tetrahydroisoquinolin-6-yl)-N-cyclopropylbenzamide;
(R)—N-cyclobutyl-4-(2-(1-cyclobutylpyrrolidin-3-yl)-1-oxo-1,2,3,4-tetrahydroisoquinolin-6-yl)benzamide;
(R)-6-(4-(azetidine-1-carbonyl)phenyl)-2-(1-cyclobutylpyrrolidin-3-yl)-3,4-dihydroisoquinolin-1(2H)-one;
(R)-4-(2-(1-cyclobutylpyrrolidin-3-yl)-1-oxo-1,2,3,4-tetrahydroisoquinolin-6-yl)-N-(2-fluoroethyl)benzamide;
(R)-4-(2-(1-cyclobutylpyrrolidin-3-yl)-1-oxo-1,2,3,4-tetrahydroisoquinolin-6-yl)-N-(2-methoxyethyl)benzamide;
4-(2-((R)-1-cyclobutylpyrrolidin-3-yl)-1-oxo-1,2,3,4-tetrahydroisoquinolin-6-yl)-N—((S)-1-methoxypropan-2-yl)benzamide;
(R)-4-(2-(1-cyclobutylpyrrolidin-3-yl)-1-oxo-1,2,3,4-tetrahydroisoquinolin-6-yl)-N-(2-isopropoxyethyl)benzamide;
(R)-2-(1-cyclobutylpyrrolidin-3-yl)-6-(4-(morpholine-4-carbonyl)phenyl)-3,4-dihydroisoquinolin-1(2H)-one;
(R)-2-(1-cyclobutylpyrrolidin-3-yl)-6-(4-(piperidine-1-carbonyl)phenyl)-3,4-dihydroisoquinolin-1(2H)-one;
(R)-2-(1-cyclobutylpyrrolidin-3-yl)-6-(2-fluoro-4-(piperidine-1-carbonyl)phenyl)-3,4-dihydroisoquinolin-1(2H)-one;
(R)-2-(1-cyclobutylpyrrolidin-3-yl)-6-(2-fluoro-4-(morpholine-4-carbonyl)phenyl)-3,4-dihydroisoquinolin-1(2H)-one;
(R)-4-(2-(1-cyclobutylpyrrolidin-3-yl)-1-oxo-1,2,3,4-tetrahydroisoquinolin-6-yl)-3-fluoro-N-(2-isopropoxyethyl)benzamide;
4-(2-((R)-1-cyclobutylpyrrolidin-3-yl)-1-oxo-1,2,3,4-tetrahydroisoquinolin-6-yl)-3-fluoro-N—((S)-1-methoxypropan-2-yl)benzamide;
(R)-4-(2-(1-cyclobutylpyrrolidin-3-yl)-1-oxo-1,2,3,4-tetrahydroisoquinolin-6-yl)-3-fluoro-N-(2-methoxyethyl)benzamide;
(R)-4-(2-(1-cyclobutylpyrrolidin-3-yl)-1-oxo-1,2,3,4-tetrahydroisoquinolin-6-yl)-3-fluoro-N-(2-fluoroethyl)benzamide;
(R)—N-cyclobutyl-4-(2-(1-cyclobutylpyrrolidin-3-yl)-1-oxo-1,2,3,4-tetrahydroisoquinolin-6-yl)-3-fluorobenzamide;
(R)-4-(2-(1-cyclobutylpyrrolidin-3-yl)-1-oxo-1,2,3,4-tetrahydroisoquinolin-6-yl)-N-cyclopropyl-3-fluorobenzamide;
(R)-4-(2-(1-cyclobutylpyrrolidin-3-yl)-1-oxo-1,2,3,4-tetrahydroisoquinolin-6-yl)-3-fluoro-N-isopropylbenzamide;
(R)-6-(4-(azetidine-1-carbonyl)-2-fluorophenyl)-2-(1-cyclobutylpyrrolidin-3-yl)-3,4-dihydroisoquinolin-1(2H)-one;
or a stereoisomer thereof or a pharmaceutically acceptable salt thereof.

Advantageously, the present invention provides a process to prepare compounds of formula I wherein X is O (Ia′) which comprises reacting a compound of formula II with a compound, R²-Hal, wherein Hal is Cl, F, Br or I in the presence of a base optionally in the presence of a solvent. The reaction is shown in scheme I.

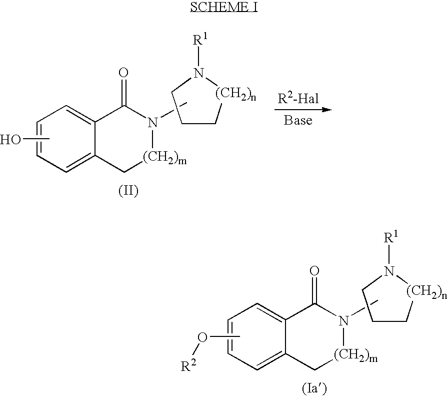

Bases suitable for use in the method of the invention include alkali metal carbonates such as Na₂CO₃, K₂CO₃, Cs₂CO₃ or the like. Solvents suitable for use in the method of the invention include alcohols such as methanol.

Compounds of formula II may be readily prepared by reacting a compound of formula III with a protected cyclic amine of formula IV to give the compound of formula V; reacting said formula V compound with a palladium catalyst such as bistriphenylphosphine palladium dichloride to give the lactam of formula VI; reacting said formula VI compound with borontribromide to give the compound of formula VII; reacting said formula VII compound with the appropriate aldehyde or ketone and NaBH₃CN to give the desired compound of formula II. The reaction is shown in reaction scheme II wherein R″ is C₁-C₄alkyl.

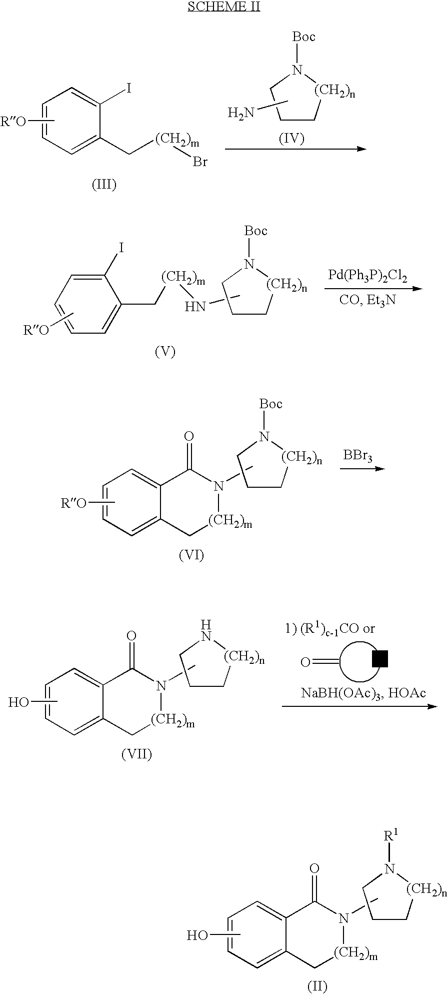

Compounds of formula I wherein X is (CR³R⁴)ₚ; p is 0; and R² is an optionally substituted aryl or heteroaryl group (Ib) may be prepared by reacting a benzoate of formula VIII with a protected azacyclylamine of formula IV to give the compound of formula IX; reacting said formula IX compound with a boronic acid of formula X in the presences of a palladium catalysts such as dichlorobis(tri-o-tolyphosphine)-palladium (II) and a base such as K₂CO₃ to give the compound of formula XI; deprotecting said formula XI compound in the presence of an acid such as trifluoroacetic acid (TFA) to give the compound of formula XII reacting said formula XII compound with an aldehyde or ketone and NaBH₃CN to give the desired compound of formula Ib. The reaction is shown in reaction scheme III wherein R″ is C₁-C₄alkyl.

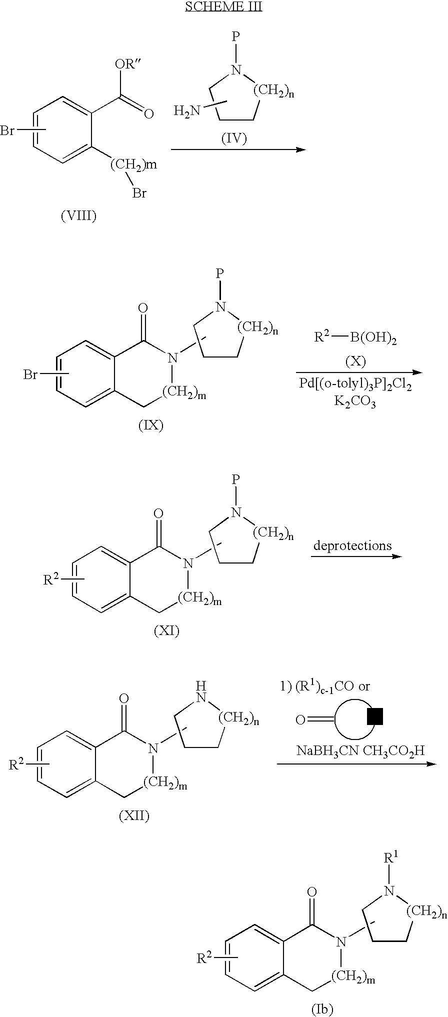

Alternatively, compounds of formula I wherein X is (CR³R⁴)ₚ; p is 0; and R² is an optionally substituted aryl or heteroaryl group (Ib) may be prepared by reacting a triflate of formula XIII with a boronic acid of formula X in the presences of a palladium catalysts, such as dichlorobis(tri-o-tolyphosphine)-palladium (II) and a base such as K₂CO₃ to give compounds of formula 1b. The reaction is shown in reaction scheme IV.

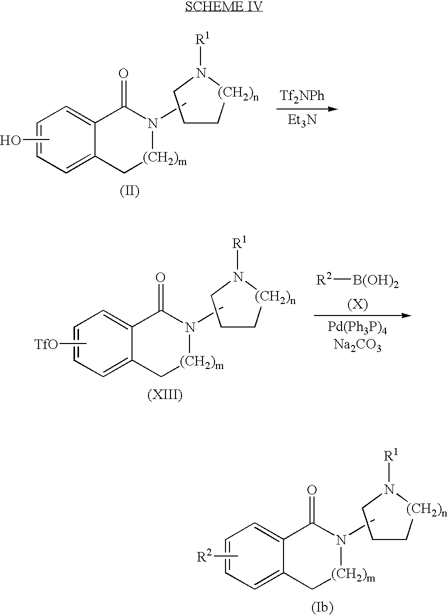

Compounds of formula II may be readily prepared by reacting a compound of formula III with a benzyl protected cyclic amine of formula XIV to give the compound of formula XV; reacting said formula XV compound with a palladium catalyst such as bistriphenylphosphine palladium dichloride to give the lactam of formula XVI; reacting said formula XVI compound with borontribromide to give the compound of formula XVII; reacting said formula XVII compound with triflate reagent, such as Tf₂NPh and a base such as triethyl amine, to generate the compound of formula XVIII, reacting said formula XVIII compound with a boronic acid of formula X in the presences of a palladium catalysts such as dichlorobis(tri-o-tolyphosphine)-palladium (II) and a base such as K₂CO₃ to give the compound of formula XIX; deprotecting said formula XIX compound in the presence of ammonium formate and palladium on carbon 10% to give the compound of formula XX, reacting said formula XX compound with an aldehyde or ketone and NaBH₃CN to give the desired compound of formula Ib. The reaction is shown in reaction scheme V.

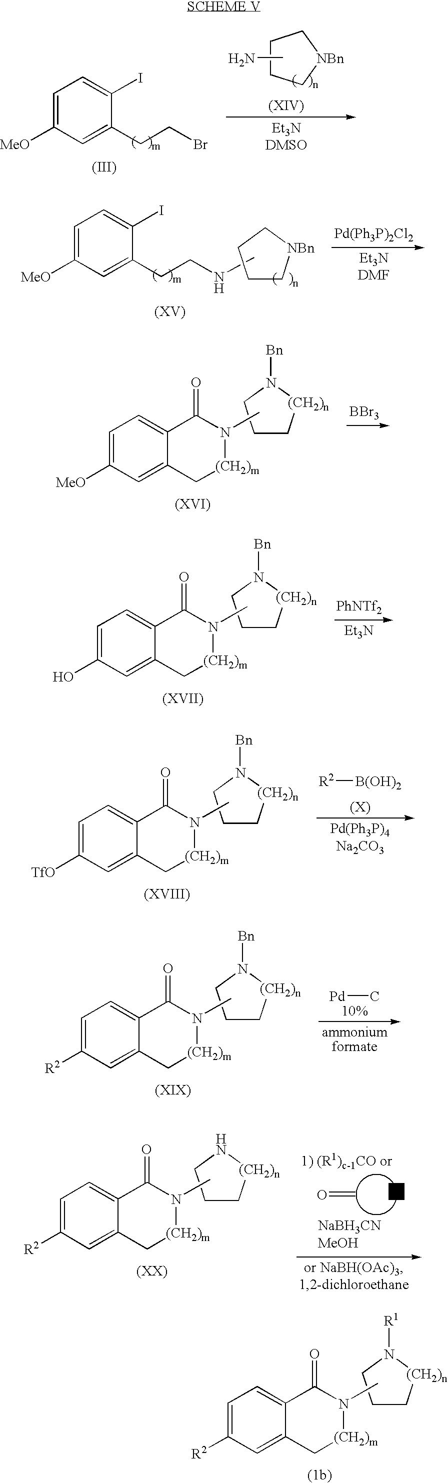

Compound of formula Ic wherein X is CO may be readily prepared by reacting a lactam of formula XIII with an amine, NR⁵R⁶, carbon monoxide, a palladium source such as dichlorobis(triphenylphosphime)palladium (II) and a base such as triethylamine to give the desired compound of formula I. The reaction is shown in scheme VI.

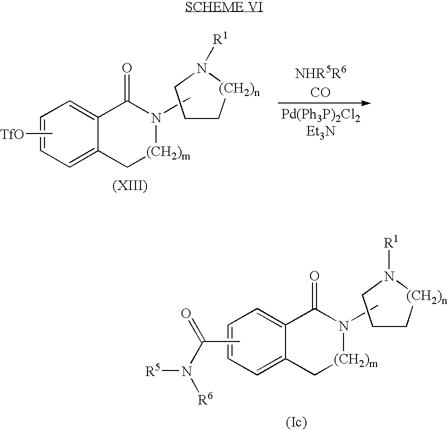

Advantageously, the formula I compounds of the invention are useful for the treatment of CNS disorders related to or affected by the Histamine-3 receptor including cognitive disorders, for example Alzheimer's disease, mild cognitive impairment, attention deficit hyperactivity disorder, schizophrenia, memory loss, obesity, sleep disorders, eating disorders, neuropathic pain or the like. Accordingly, the present invention provides a method for the treatment of a disorder of the central nervous system related to or affected by the Histamine-3 receptor in a patient in need thereof which comprises providing said patient a therapeutically effective amount of a compound of formula I as described hereinabove. The compounds may be provided by oral or parenteral administration or in any common manner known to be an effective administration of a therapeutic agent to a patient in need thereof.

The term “providing” as used herein with respect to providing a compound or substance embraced by the invention, designates either directly administering such a compound or substance, or administering a prodrug, derivative or analog which forms an equivalent amount of the compound or substance within the body.

The inventive method includes: a method for the treatment of schizophrenia; a method for the treatment of a disease associated with a deficit in memory, cognition or learning or a cognitive disorder such as Alzheimer's disease or attention deficit hyperactivity disorder; a method for the treatment of a mild cognitive disorder, a method for the treatment of a developmental disorder such as schizophrenia; a method for the treatment of a sleep disorder, a method for the treatment of an eating disorder, a method for the treatment of neuropathic pain or any other CNS disease or disorder associated with or related to the H₃ receptor.

In one embodiment, the present invention provides a method for treating attention deficit hyperactivity disorders (ADHD, also known as Attention Deficit Disorder or ADD) in both children and adults. Accordingly, in this embodiment, the present invention provides a method for treating attention deficit disorders in a pediatric patient.

The present invention therefore provides a method for the treatment of each of the conditions listed above in a patient, preferably in a human, said method comprises providing said patient a therapeutically effective amount of a compound of formula I as described hereinabove. The compounds may be provided by oral or parenteral administration or in any common manner known to be an effective administration of a therapeutic agent to a patient in need thereof.

The therapeutically effective amount provided in the treatment of a specific CNS disorder may vary according to the specific condition(s) being treated, the size, age and response pattern of the patient, the severity of the disorder, the judgment of the attending physician and the like. In general, effective amounts for daily oral administration may be about 0.01 to 1,000 mg/kg, preferably about 0.5 to 500 mg/kg and effective amounts for parenteral administration may be about 0.1 to 100 mg/kg, preferably about 0.5 to 50 mg/kg.

In actual practice, the compounds of the invention are provided by administering the compound or a precursor thereof in a solid or liquid form, either neat or in combination with one or more conventional pharmaceutical carriers or excipients. Accordingly, the present invention provides a pharmaceutical composition which comprises a pharmaceutically acceptable carrier and an effective amount of a compound of formula I as described hereinabove.

In one embodiment, the invention relates to compositions comprising at least one compound of formula I, or a pharmaceutically acceptable salt thereof, and one or more pharmaceutically acceptable carriers, excipients, or diluents. Such compositions include pharmaceutical compositions for treating or controlling disease states or conditions of the central nervous system. In certain embodiments, the compositions comprise mixtures of one or more compounds of formula I.

In certain embodiments, the invention relates to compositions comprising at least one compound of formula I, or a pharmaceutically acceptable salt thereof, and one or more pharmaceutically acceptable carriers, excipients, or diluents. Such compositions are prepared in accordance with acceptable pharmaceutical procedures. Pharmaceutically acceptable carriers are those carriers that are compatible with the other ingredients in the formulation and are biologically acceptable.

The compounds of formula I may be administered orally or parenterally, neat, or in combination with conventional pharmaceutical carriers. Applicable solid carriers can include one or more substances that can also act as flavoring agents, lubricants, solubilizers, suspending agents, fillers, glidants, compression aids, binders, tablet-disintegrating agents, or encapsulating materials. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

In certain embodiments, a compound of formula I is provided in a disintegrating tablet formulation suitable for pediatric administration.

Liquid carriers can be used in preparing solutions, suspensions, emulsions, syrups and elixirs. The active ingredient can be dissolved or suspended in a pharmaceutically acceptable liquid carrier such as water, an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

In certain embodiments, a liquid pharmaceutical composition is provided wherein said composition is suitable for pediatric administration. In other embodiments, the liquid composition is a syrup or suspension.

Liquid pharmaceutical compositions that are sterile solutions or suspensions can be administered by, for example, intramuscular, intraperitoneal or subcutaneous injection. Sterile solutions can also be administered intravenously. Compositions for oral administration can be in either liquid or solid form.

The compounds of formula I may be administered rectally or vaginally in the form of a conventional suppository. For administration by intranasal or intrabronchial inhalation or insufflation, the compounds of formula I can be formulated into an aqueous or partially aqueous solution, which can then be utilized in the form of an aerosol. The compounds of formula I can also be administered transdermally through the use of a transdermal patch containing the active compound and a carrier that is inert to the active compound, is non-toxic to the skin, and allows delivery of the agent for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active ingredient can also be suitable. A variety of occlusive devices can be used to release the active ingredient into the blood stream such as a semipermeable membrane covering a reservoir containing the active ingredient with or without a carrier, or a matrix containing the active ingredient. Other occlusive devices are known in the literature.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form.

The therapeutically effective amount of a compound of formula I provided to a patient will vary depending upon what is being administered, the purpose of the administration, such as prophylaxis or therapy, the state of the patient, the manner of administration, or the like. In therapeutic applications, compounds of formula I are provided to a patient suffering from a condition in an amount sufficient to treat or at least partially treat the symptoms of the condition and its complications. An amount adequate to accomplish this is a “therapeutically effective amount” as described previously herein. The dosage to be used in the treatment of a specific case must be subjectively determined by the attending physician. The variables involved include the specific condition and the size, age, and response pattern of the patient. Generally, a starting dose is about 5 mg per day with gradual increase in the daily dose to about 150 mg per day, to provide the desired dosage level in the patient.

In certain embodiments, the present invention is directed to prodrugs of compounds of formula I. The term “prodrug,” as used herein, means a compound that is convertible in vivo by metabolic means (e.g. by hydrolysis) to a compound of formula I. Various forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991), Bundgaard, et al., Journal of Drug Delivery Reviews, 8:1-38(1992), Bundgaard, J. of Pharmaceutical Sciences, 77:285 et seq. (1988); and Higuchi and Stella (eds.) Prodrugs as Novel Drug Delivery Systems, American Chemical Society (1975).

For a more clear understanding, and in order to illustrate the invention more clearly, specific examples thereof are set forth hereinbelow. The following examples are merely illustrative and are not to be understood as limiting the scope and underlying principles of the invention in any way. The terms HPLC and NMR designate high performance liquid chromatography and proton nuclear magnetic resonance, respectively. The term MS designates mass spectroscopy with (+) referring to the positive mode which generally gives a M+1 (or M+H) absorption where M=the molecular mass. All compounds are analyzed at least by MS and NMR. Unless otherwise noted, all parts are parts by weight.

### EXAMPLES

### Example 1

### Preparation of methyl 4-{[2-(1-Cyclobutylpiperidin-4-yl)-1-oxo-1,2,3,4-tetrahydroisoquinolin-6-yl]oxy}benzoate

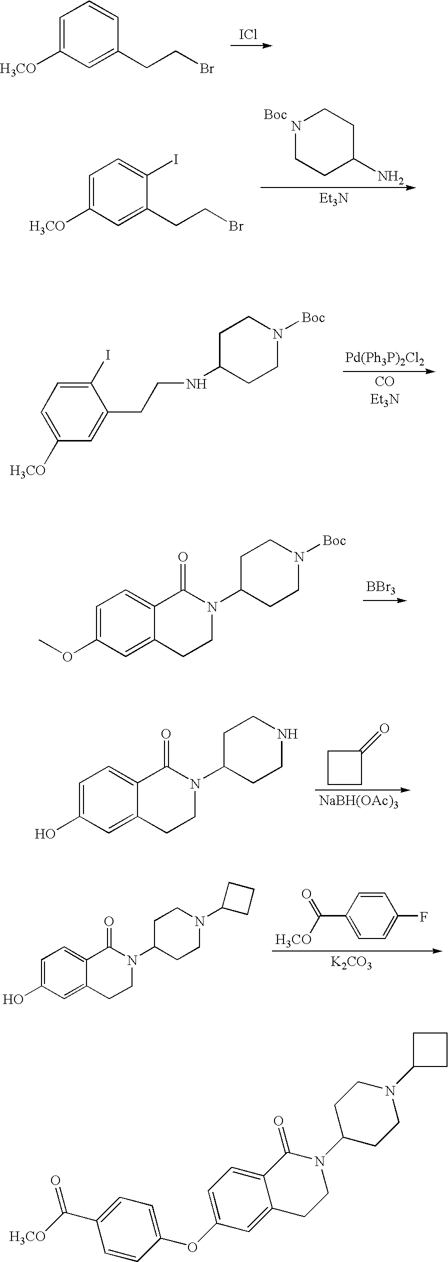

### Step 1: 2-(2-Bromoethyl)-1-iodo-4-methoxybenzene

To a solution of 3-methyloxyphenyl ethyl bromide (1.0 g, 4.6 mmol) in methanol (20 mL) at room temperature was added iodine monochloride (0.75 g, 4.6 mmol) and the mixture was stirred at room temperature overnight. The solvents were removed in vacuo. The residue was dissolved in methylene chloride and washed with sodium sulfite (saturated solution, 3×100 mL). The organic layer was collected, dried (sodium sulfate) and concentrated in vacuo. The residue was purified by ISCO CombiFlash® chromatography (silica, 0-10% ethyl acetate in hexanes) to afford 1.4 g (90%) of 2-(2-bromoethyl)-1-iodo-4-methoxybenzene as a colorless oil. MS (EI) m/z 340 [M]⁺.

### Step 2: tert-Butyl 4-(2-iodo-5-methoxyphenethylamino)piperidine-1-carboxylate

To a solution of 2-(2-bromoethyl)-1-iodo-4-methoxybenzene (2.1 g, 6.1 mmol) in DMSO at room temperature was added tert-butyl 4-aminopiperidine-1-carboxylate (1.9 g, 9.2 mmol) and triethyl amine (2.5 mL, 18 mmol) and the reaction mixture was heated at 40° C. overnight. The reaction mixture was cooled to room temperature and partitioned between dichloromethane and water. The aqueous phase was extracted with dichloromethane (3×100 mL). The combined organic phases were washed with water (3×100 mL), dried (sodium sulfate) and concentrated in vacuo. The residue was purified by ISCO CombiFlash® chromatography (silica, 0-10% methanol in dichloromethane plus 0.5% ammonium hydroxide) to afford 1.0 g (34%) of tert-butyl 4-(2-iodo-5-methoxyphenethylamino)piperidine-1-carboxylate as a colorless oil. MS (ES) m/z 461.0 [M+H]⁺.

### Step 3: tert-Butyl 4-(6-methoxy-1-oxo-3,4-dihydroisoquinolin-2(1H)-yl)piperidine-1-carboxylate

To a solution of tert-butyl 4-(2-iodo-5-methoxyphenethy-amino)piperidine-1-carboxylate (0.32 g, 0.7 mmol) in N,N-dimethylformamide was added dichlorobistri-phenylphosphine palladium (II) (24 mg, 0.03 mmol) and triethylamine (0.28 mL, 2.1 mmol) and the mixture was purged with CO (balloon). The reaction mixture was heated at 90° C. under CO atmosphere for 4-6 hrs (monitored by LC-MS). The reaction mixture was cooled to room temperature and filtered through a pad of celite, the filtrate was partitioned between water (100 mL) and dichloromethane (100 mL). The aqueous phase was washed with dichloromethane (3×100 mL). The combined organic layers were washed with water (3×100 mL), dried (sodium sulfate) and the solvent was removed in vacuo. The residue was purified by ISCO CombiFlash® chromatography (silica, 0-10% methanol in methylene with 0.5% ammonium hydroxide) to afford 0.16 g (64%) of tert-butyl 4-(6-methoxy-1-oxo-3,4-dihydroisoquinolin-2(1H)-yl)piperidine-1-carboxylate as a light brown oil. MS (ES) m/z 361 [M+H]⁺.

### Step 4: 6-Hydroxy-2-(piperidin-4-yl)-3,4-dihydroisoquinolin-1(2H)-one

To a solution of tert-butyl 4-(6-methoxy-1-oxo-3,4-dihydroisoquinolin-2(1H)-yl)piperidine-1-carboxylate (0.36 g, 1.0 mmol) at −78° C. was added boron tribromide (0.23 mL, 2.5 mmol) and the reaction mixture was stirred at room temperature overnight. The reaction mixture was cooled to 0° C. and quenched with methanol until all the solids were dissolved. The reaction mixture was neutralized (PH 7.0) with sodium hydroxide (2.5 N) and extracted with methylene chloride (many times) until no products were detected from aqueous layer by LC-MS. The combined organic layers were concentrated in vacuo. Purification by ISCO CombiFlash® chromatography (silica, 0-15% methanol in dichloromethane) afforded 0.24 g (100%) of 6-hydroxy-2-(piperidin-4-yl)-3,4-dihydroisoquinolin-1(2H)-one as a thick oil. MS (ES) m/z 247 [M+H]⁺.

### Step 5: 2-(1-Cyclobutylpiperidin-4-yl)-6-hydroxy-3,4-dihydroisoquinolin-1(2H)-one

To a solution of 6-hydroxy-2-(piperidin-4-yl)-3,4-dihydroisoquinolin-1(2H)-one (0.26 g, 1.0 mmol) in 1,2-dichloroethane-methanol at room temperature was added cyclobutanone (2.0 mL), sodium triacetoxyborohydride (0.33 g, 1.6 mmol) and acetic acid (0.15 mL, 2.6 mmol) and the reaction mixture was allowed to stir at room temperature overnight. The reaction was quenched by the addition of aqueous sodium hydroxide (10 mL, 1.0 N) and the mixture was partitioned between dichloromethane and water. The aqueous phase was extracted with dichloromethane (3×100 mL). The organic layers were combined and concentrated in vacuo. The residue was purified by ISCO CombiFlash® chromatography (silica gel, 0-10% methanol in dichloromethane with 0.5% ammonium hydroxide) to afford 0.26 g (85%) of 2-(1-cyclobutylpiperidin-4-yl)-6-hydroxy-3,4-dihydroisoquinolin-1(2H)-one as a colorless oil. The oil was dissolved in ethanol and made into its hydrochloride salt as a white solid. mp decomposed at 300° C.; MS (ES) m/z 301.2; HRMS: calcd for C₁₈H₂₄N₂O₂+H+, 301.19105; found (ESI, [M+H]+ Obs'd), 301.1915.

### Step 6: Methyl 4-(2-(1-cyclobutylpiperidin-4-yl)-1-oxo-1,2,3,4-tetrahydro-isoquinolin-6-yloxy)benzoate

To a solution of 2-(1-cyclobutylpiperidin-4-yl)-6-hydroxy-3,4-dihydroisoquinolin-1(2H)-one (0.4 g, 1.3 mmol) in anhydrous DMF 15 mL) in a pressure vessel at room temperature was added potassium carbonate (0.46 g, 3.3 mmol) and methyl-4-fluorobenzoate (0.41 g, 2.7 mmol) and reaction mixture was allowed to heat at 110° C. for 22 hours. The reaction mixture was cooled to room temperature and partitioned between dichloromethane (100 mL) and water. The aqueous phase was extracted with dichloromethane (3×150 mL). The combined organic layers were washed with water (3×100 mL), dried (sodium sulfate) and the solvent was removed in vacuo. Purification by ISCO CombiFlash® chromatography (silica, 0-10% methanol/dichloromethane plus 0.5% ammonium hyderoxide) provided 0.37 g (65%) of methyl 4-{[2-(1-cyclobutylpiperidin-4-yl)-1-oxo-1,2,3,4-tetrahydroisoquinolin-6-yl]-oxy}benzoate as colorless oil. The oil was dissolved in ethanol and made into its hydrochloride salt as a white solid. mp 269-270° C.; MS (ES) m/z 435.3.

### Example 2

### Preparation of 4-{[2-(1-cyclobutylpiperidin-4-yl)-1-oxo-1,2,3,4-tetrahydroiso-quinolin-6-yl]oxy}benzoic acid

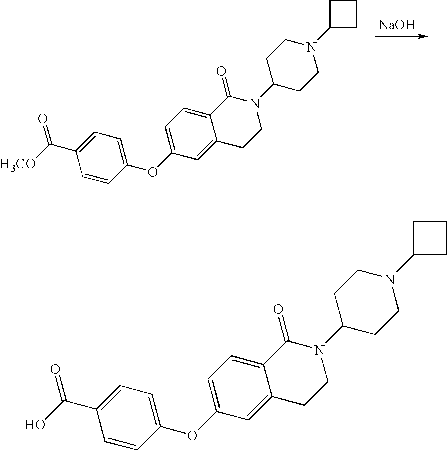

A solution of methyl 4-{[2-(1-cyclobutylpiperidin-4-yl)-1-oxo-1,2,3,4-tetrahydroisoquinolin-6-yl]oxy}benzoate (0.24 g, 0.55 mmol) in ethanol at room temperature was treated with aqueous sodium hydroxide (2.5 N, 6.0 mL), stirred at room temperature for 2 h, carefully neutralized to pH 7 with hydrochloric acid (2.0 N) and filtered. The filtercake was washed with water, dried under vacuum at 78° C. overnight to give the title product as a white solid, 0.91 g (91%), mp 255-256° C., identified by NMR and mass spectral analyses. MS (ES) m/z 419.3; HRMS: calcd for C25H28N2O4+H+, 421.21218; found (ESI, [M+H]+ Obs'd), 421.2127.

### Example 3

### Preparation of 4-{[2-(1-cyclobutylpiperidin-4-yl)-1-oxo-1,2,3,4-tetrahydroiso-quinolin-6-yl]oxy}benzamide Hydrochloride

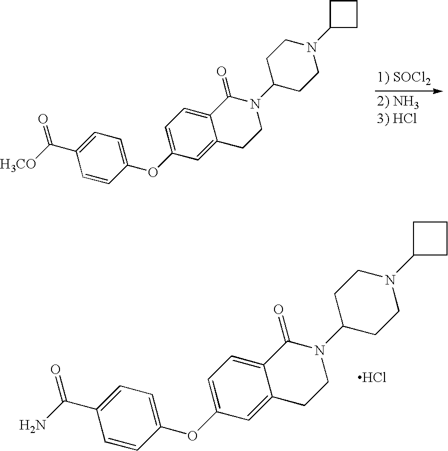

A mixture of thionyl chloride (4.0 mL) and 4-{[2-(1-cyclobutylpiperidin-4-yl)-1-oxo-1,2,3,4-tetrahydroisoquinolin-6-yl]oxy}benzoic acid (40 mg, 0.095 mmol) was stirred at reflux for 0.5 hour. The reaction mixture was concentrated in vacuo to afford a crude acid chloride. The acid chloride was dissolved in tetrahydrofuran (10 mL) and cooled to 0° C., then NH₃ was bubbled through for 2 minutes. The mixture was allowed to warm to room temperature and stirred for 0.5 h. The reaction mixture was partitioned between methylene chloride and 1 N aqueous sodium hydroxide. The aqueous layer was washed with methylene chloride (3×100 mL). The organic layers were combined, dried (anhydrous sodium sulfate) and the solvent was concentrated in vacuo. The residue was purified by ISCO CombiFlash® chromatography (silica gel, 0-10% methanol in methylene with 0.5% ammonium hydroxide) to afford the free amine of the title compound as a colorless oil. The oil was dissolved in ethanol, treated with ethereal HCl, stirred for 10 min. and concentrated to dryness under vacuum to provide the title product as a white solid, 33 mg (82%), mp 309-310° C.; identified by NMR and mass spectral analyses. MS (ES) m/z 420.2; HRMS: calcd for C₂₅H₂₉N₃O₃+H+, 420.22817; found (ESI, [M+H]+ Obs'd), 420.2285.

### Examples 4-12

### Preparation of 4-{[2-(1-cyclobutylpiperidin-4-yl)-1-oxo-1,2,3,4-tetrahydroiso-quinolin-6-yl]oxy}benzamide Hydrochloride Compounds

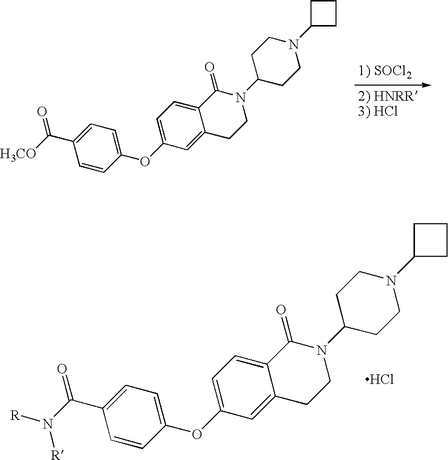

Using essentially the same procedure described in Example 3 and employing the desired amine, the compounds shown in Table I were obtained and identified by NMR and mass spectral analyses.

TABLE I

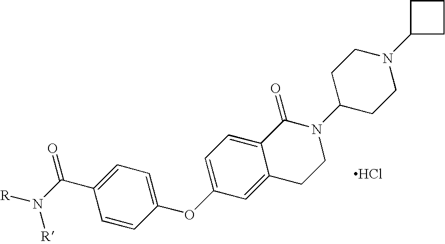

| Ex. No. | R | R′ | mp ° C. | [M + H] |
|---|---|---|---|---|
| 4 | H | methyl | >310 | 434.2 |
| 5 | H | ethyl | 317-318 | 448.3 |
| 6 | H | isopropyl | 309-311 | 462.3 |
| 7 | H | cyclopropyl | 307-309 | 460.3 |
| 8 | H | cyclobutyl | >300 | 474.3 |
| 9 | H | cyclopentyl | >300 | 488.3 |
| 10 | CH₃ | CH₃ | 262-264 | 448.3 |
| 11 | C₂H₅ | C₂H₅ | 249-251 | 476.3 |

| | | | |
|---|---|---|---|
| 12 | cyclopentyl | 253-255 | 474.3 |

### Example 13

### Preparation of methyl 4-({[2-(1-Cyclobutylpiperidin-4-yl)-1-oxo-1,2,3,4-tetrahydroisoquinolin-6-yl]oxy}methyl)benzoate

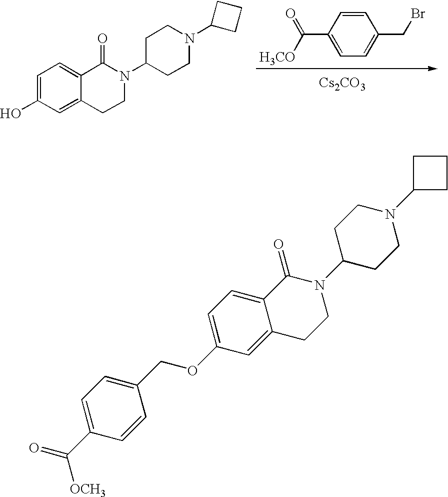

A solution of 2-(1-cyclobutylpiperidin-4-yl)-6-hydroxy-3,4-dihydroisoquinolin-1(2H)-one (0.84 g, 2.8 mmol) in acetone at room temperature was treated with cesium carbonate (1.8 g, 5.6 mmol) and methyl 4-(bromomethyl)benzoate (0.96 g, 4.2 mmol), was heated at 50° C. for 16 h, cooled to room temperature and partitioned between dichloromethane and water. The aqueous phase was extracted with methylene chloride. The combined organic layers were washed with water, dried (sodium sulfate) and concentrated in vacuo. the residue was purified by ISCO CombiFlash® chromatography (silica, 0-10% methanol/dichloromethane plus 0.5% ammonium hydroxide) to provide the title compound as a white solid, 1.0 g (80%), mp 165-166° C.; identified by NMR and mass spectral analyses. MS (ES) m/z 449.3; HRMS: calcd for C27H32N2O4+H+, 449.24348; found (ESI, [M+H]+ Obs'd), 449.2439.

### Example 14

### Preparation of 4-({[2-(1-cyclobutylpiperidin-4-yl)-1-oxo-1,2,3,4-tetrahydroiso-quinolin-6-yl]oxy}methyl)benzoic acid

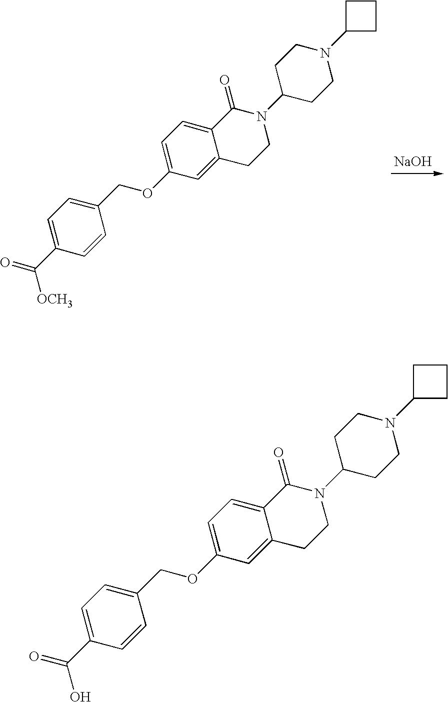

A solution of methyl 4-({[2-(1-cyclobutylpiperidin-4-yl)-1-oxo-1,2,3,4-tetrahydroisoquinolin-6-yl]oxy}methyl)benzoate (0.7 g, 1.6 mmol) in ethanol at room temperature is treated with aqueous sodium hydroxide (2.5 N, 15 mL), stirred at room temperature for 2 h, neutralized with hydrochloric acid (2.0 N) and filtered. The filtercake was washed with water, dried under vacuum at 78° C. overnight to afford the title compound as a white solid, 0.61 g (90%), mp 269-270° C.; identified by NMR and mass spectral analyses. MS (ES) m/z 433.3; HRMS: calcd for C₂₆H₃₀N₂O₄+H⁺, 435.22783; found (ESI, [M+H]+ Obs'd), 435.2279.

### Example 15

### Preparation of 4-({[2-(1-cyclobutylpiperidin-4-yl)-1-oxo-1,2,3,4-tetrahydroiso-quinolin-6-yl]oxy}methyl)benzamide acid hydrochloride

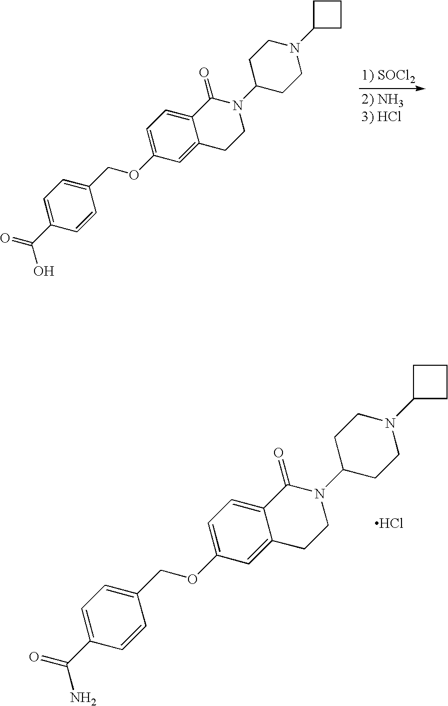

A mixture of thionyl chloride (5 mL) and 4-({[2-(1-cyclobutylpiperidin-4-yl)-1-oxo-1,2,3,4-tetrahydroisoquinolin-6-yl]oxy}methyl)benzoic acid (40 mg, 0.09 mmol) was stirred at reflux temperature for 0.5 hour. The reaction mixture was concentrated in vacuo to afford a crude acid chloride. The acid chloride was dissolved in tetrahydrofuran and cooled to 0° C., then NH₃ was bubbled through for 2 minutes. The mixture was allowed to warm to room temperature, stirred for 0.5 h and partitioned between methylene chloride and 1 N aqueous sodium hydroxide. The aqueous layer was extracted with methylene chloride. The extracts were combined with the organic phase, dried (anhydrous sodium sulfate) and concentrated in vacuo. The residue was purified by ISCO CombiFlash® chromatography (silica gel, 0-10% methanol in methylene with 0.5% ammonium hydroxide) to afford the free amine of the title compound as a colorless oil. The oil was dissolved in ethanol treated with etheral HCl, stirred for 10 min. and concentrated to dryness to afford the title compound as a white solid, 34 mg (86%), mp 292-293° C.; Identified by NMR and mass spectral analyses. MS (ES) m/z 434.2; HRMS: calcd for C₂₆H₃₁N₃O₃+H⁺, 434.24382; found (ESI, [M+H]⁺ Obs'd), 434.2440;

### Examples 16-21

### Preparation of 4-({[2-(1-cyclobutylpiperidin-4-yl)-1-oxo-1,2,3,4-tetrahydroiso-quinolin-6-yl]oxy}methyl)benzoic acid hydrochloride compounds

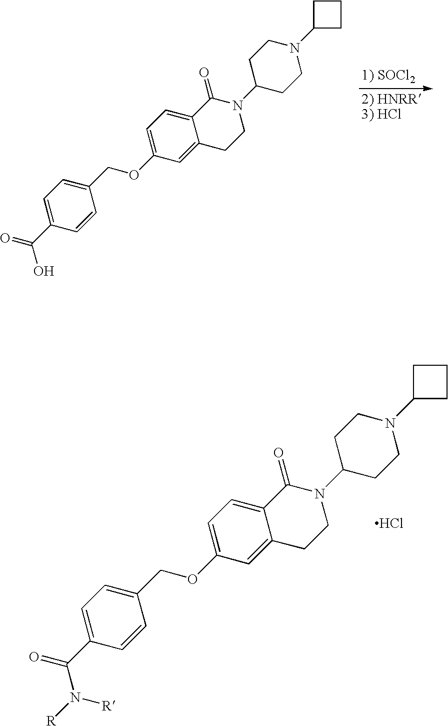

Using essentially the same procedure described in Example 15 and employing the desired amine, the compounds shown in Table II were obtained and identified by NMR and mass spectral analyses.

TABLE II

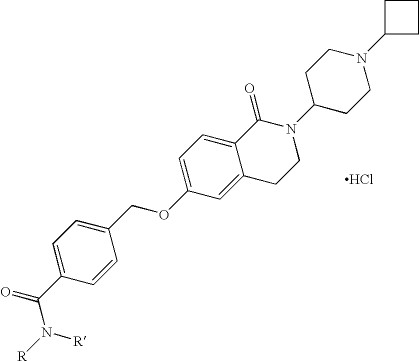

| Ex. No. | R | R′ | mp ° C. | [M + H] |
|---|---|---|---|---|
| 16 | H | methyl | 302-303 | 448.2 |
| 17 | H | ethyl | 307-309 | 462.2 |
| 18 | CH₃ | CH₃ | 292-294 | 462.1 |
| 19 | H | cyclobutyl | 286-288 | 488.2 |
| 20 | cyclopentyl | | 284-286 | 488.2 |
| 21 | H | cyclopentyl | 289-291 | 502.2 |

### Example 22

### Preparation of 4-(1-oxo-2-piperidin-4-yl-2,3-dihydro-1H-isoindol-5-yl)benzonitrile hydrochloride

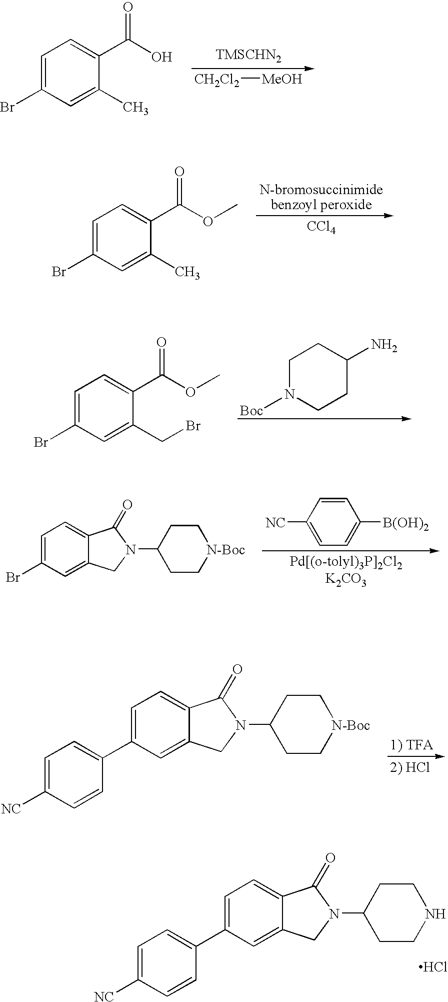

### Step 1: Methyl 4-bromo-2-methylbenzoate

A suspension of 4-bromo-2-methylbenzoic acid (4.98 g, 23 mmol) in dichloromethane and methanol at 0° C. was treated with a solution of trimethylsilyl-diazomethane (11.6 ml, 2.0 M solution in hexane), stirred at 0° C. for 3 h, allowed to warm to room temperature, diluted with 1.0 N sodium hydroxide and extracted with dichloromethane. The combined extracts were dried over Na₂SO₄ and concentrated in vacuo. The residue was purified by ISCO CombiFlash® chromatography (silica, 0-5% ethyl acetate in hexanes) to provide 4.72 g (89%) of methyl 4-bromo-2-methylbenzoate as a colorless oil. MS (EI) m/z 228 [M]⁺.

### Step 2: Methyl 4-bromo-2-(bromomethyl)benzoate

A solution of methyl 4-bromo-2-methylbenzoate (1.0 g, 4.3 mmol) in carbon tetrachloride was treated with N-bromosuccinimide (0.93 g, 5.2 mmol) and benzoyl peroxide (0.53 g, 2.2 mmol), heated at 85° C. for 5 h, cooled to room temperature and filtered. The filtercake was washed with methylene chloride. The combined filtrates were concentrated in vacuo. The residue was purified by ISCO CombiFlash® chromatography (silica, 0-5% ethyl acetate in hexanes) to afford 1.59 g (74%) of methyl 4-bromo-2-(bromomethyl)benzoate as a light yellow oil. MS (EI) m/z 308 [M]⁺.

### Step 3: tert-Butyl 4-(5-bromo-1-oxoisoindolin-2-yl)piperidine-1-carboxylate

A mixture of methyl 4-bromo-2-(bromomethyl)benzoate (1.45 g, 4.7 mmol), tert-butyl 4-aminopiperidine-1-carboxylate (1.40 g, 7.0 mmol) and diisopropylamine (2.0 mL, 11.98 mmol) in methanol was heated at 65° C. for 18 hours, cooled to room temperature, diluted with methylene chloride, washed with aqueous hydrochloric acid, dried (sodium sulfate) and concentrated in vacuo. The residue was purified by ISCO CombiFlash® chromatography (silica, 0-75% ethyl acetate in hexanes) to afford 1.0 g (53%) of tert-butyl 4-(5-bromo-1-oxoisoindolin-2-yl)piperidine-1-carboxylate as a white solid, mp 179-180° C.; MS (ES) m/z 395.1 [M+H]⁺.

### Step 4: tert-Butyl 4-(5-(4-cyanophenyl)-1-oxoisoindolin-2-yl)piperidine-1-carboxylate

A solution of tert-butyl 4-(5-bromo-1-oxoisoindolin-2-yl)piperidine-1-carboxylate (0.9 g, 2.3 mmol) and 4-cyanobenzene boronic acid (1.4 g, 9.1 mmol) in dioxane at 90° C. was treated with dichlorobis(tri-o-tolyphosphine)-palladium (II) (89 mg, 0.11 mmol), potassium carbonate (0.78 g, 5.7 mmol) and water, heated at 90° C. for 0.5 h, cooled to room temperature and filtered through a pad of celite. The filtrate was diluted with 1N sodium hydroxide and extracted with dichloromethane. The combined extracts were concentrated in vacuo. The residue was purified by ISCO CombiFlash® chromatography (silica gel, 0-10% methanol in dichloromethane with 0.5% ammonium hydroxide) to afford 0.81 g (85%) of the title compound as a colorless oil. MS (ES) m/z 418.2 [M+H]⁺.

### Step 5: 4-(1-oxo-2-(Piperidin-4-yl)isoindolin-5-yl)benzonitrile hydrochloride

A solution of tert-butyl 4-(5-(4-cyanophenyl)-1-oxoisoindolin-2-yl)piperidine-1-carboxylate (0.32 g, 0.76 mmol) in dichloromethane at room temperature was treated with trifluoroacetic acid (4 mL), stirred at room temperature for 3 h, diluted with in excess 1N sodium hydroxide and extracted with methylene chloride. The extracts were combined, dried over sodium sulfate and concentrated in vacuo. The residue was purified by ISCO CombiFlash® chromatography (silica gel, 0-10% methanol in dichloromethane with 0.5% ammonium hydroxide) to afford 0.22 g (100%) of the free amine of the title compound as a colorless oil. The oil was dissolved in ethanol, treated with ethereal HCl, stirred for 10 min. and concentrated to dryness to afford the title compound as a white solid, mp 105-106° C.; identified by NMR and mass spectral analyses. MS (ES) m/z 316.2; HRMS: calcd for C₂₀H₁₉N₃O+H⁺, 318.16009; found (ESI, [M+H]⁺ Obs'd), 318.1602.

### Example 23

### Preparation of 4-[2-(1-ethylpiperidin-4-yl)-1-oxo-2,3-dihydro-1H-isoindol-5-yl]benzonitrile hydrochloride

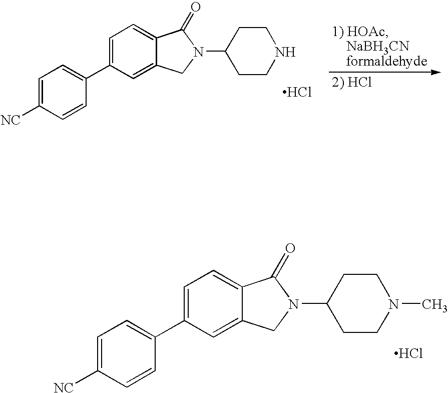

A solution of 4-(1-oxo-2-(piperidin-4-yl)isoindolin-5-yl)benzonitrile (60 mg, 0.19 mmol) in methanol at room temperature was added formaldehyde (1.0 mL), sodium cyanoborohydride (18 mg, 0.28 mmol) and acetic acid (27 μL, 0.47 mmol), stirred at room temperature overnight, quenched with 1.0 N sodium hydroxide, diluted with water and extracted with dichloromethane. The extracts were combined and concentrated in vacuo. The residue was purified by ISCO CombiFlash® chromatography (silica gel, 0-10% methanol in dichloromethane with 0.5% ammonium hydroxide) to afford 60 mg (96%) of the free amine of the title compound as a colorless oil. The oil was dissolved in ethanol, treated with ethereal HCl, stirred for 10 min. and concentrated to dryness to afford the title compound as a white solid, mp 322-323° C.; identified by NMR and mass spectral analyses. MS (APPI) m/z 332; HRMS: calcd for C₂₁H₂₁N₃O+H⁺, 332.17574; found (ESI, [M+H]⁺ Obs'd), 332.1760.

### Examples 24-30

### Preparation of 4-[2-(1-ethylpiperidin-4-yl)-1-oxo-2,3-dihydro-1H-isoindol-5-yl]benzonitrile hydrochloride

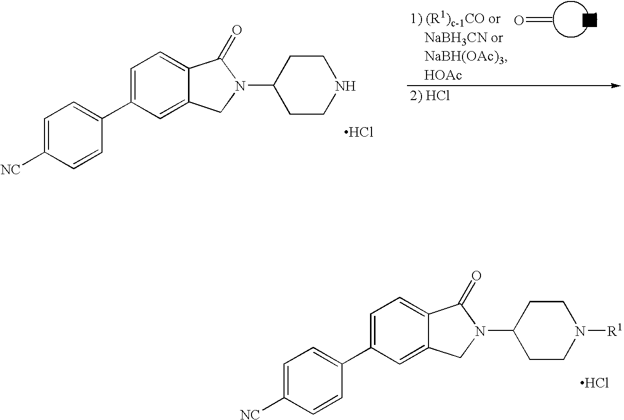

Using essentially the same procedure described in Example 23 and employing the desired aldehyde or ketone, the compounds shown in Table III were obtained and identified by NMR and mass spectral analyses.

TABLE III

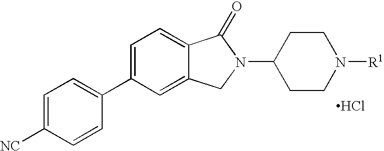

| Ex. No. | R¹ | mp ° C. | [M + H] |
|---|---|---|---|
| 24 | ethyl | >300 | 346 |
| 25 | propyl | 300-302 | 360 |
| 26 | cyclopropylmethyl | 309-310 | 372.2 |
| 27 | cyclopentylmethyl | >300 | 400 |
| 28 | cyclobutyl | 320 dec | 372.2 |
| 29 | cyclopentyl | 270 dec | 386.2 |
| 30 | cyclohexyl | >300 | 400.3 |

### Example 31

### Preparation of (R)-6-hydroxy-2-(pyrrolidin-3-yl)-3,4-dihydroisoquinolin-1(2H)-one

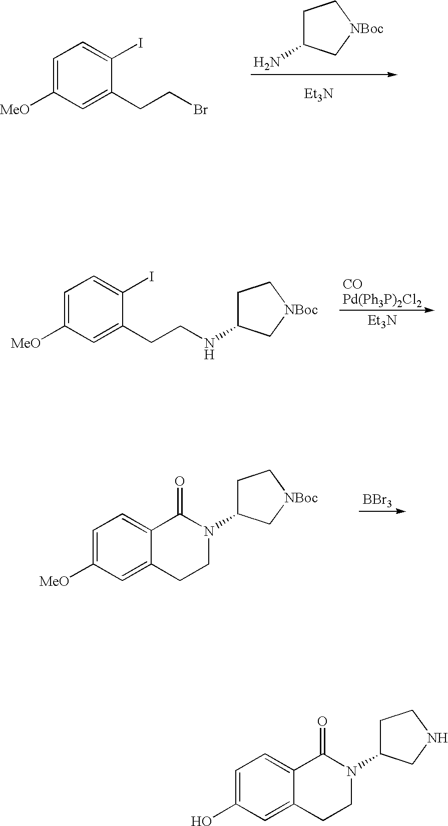

### Step 1: (R)-tert-Butyl 3-(2-Iodo-5-methoxyphenethylamino)pyrrolidine-1-carboxylate

Using essentially the same procedure described in Step 2 of Example 1 and employing the (R)-tert-butyl 3-aminopyrrolidine-1-carboxylate (3.0 g, 16 mmol), the title product of (R)-tert-butyl 3-(2-iodo-5-methoxyphenethylamino)pyrrolidine-1-carboxylate (1.66 g, 36%) was obtained as a light brown oil, [α]_{D}²⁵=4° (c=1% SOLUTION, MeOH); MS (ES) m/z 447.0 [M+H]⁺.

### Step 2: (R)-tert-Butyl 3-(6-methoxy-1-oxo-3,4-dihydroisoquinolin-2(1H)-yl)pyrrolidine-1-carboxylate

Using essentially the same procedure described in Step 3 of Example 1 and employing (R)-tert-butyl 3-(2-iodo-5-methoxyphenethylamino)pyrrolidine-1-carboxylate (1.65 g, 3.7 mmol), the title product of (R)-tert-butyl 3-(6-methoxy-1-oxo-3,4-dihydroisoquinolin-2(1H)-yl)pyrro-lidine-1-carboxylate (0.98 g, 77%) was obtained as a light brown oil, MS (ES) m/z 347.1 [M+H]⁺.

### Step 3: (R)-6-Hydroxy-2-(pyrrolidin-3-yl)-3,4-dihydroisoquinolin-1(2H)-one

Using essentially the same procedure described in Step 4 of Example 1 and employing (R)-tert-butyl 3-(6-methoxy-1-oxo-3,4-dihydroisoquinolin-2(1H)-yl)pyrro-lidine-1-carboxylate (0.98 g, 2.8 mmol), the title product of (R)-6-hydroxy-2-(pyrrolidin-3-yl)-3,4-dihydroisoquinolin-1(2H)-one (0.59 g, 91%) can be obtaianed as awhite solid. mp [α]_{D}²⁵=28° (c=1% SOLUTION, MeOH); HRMS: calcd for C13H16N2O2+H+, 233.12845; found (ESI, [M+H]+ Obs'd), 233.1288;

### Example 32

### Preparation of (R)-4-(2-(1-cyclobutylpyrrolidin-3-yl)-1-oxo-1,2,3,4-tetrahydroiso-quinolin-6-yloxy)benzoic acid

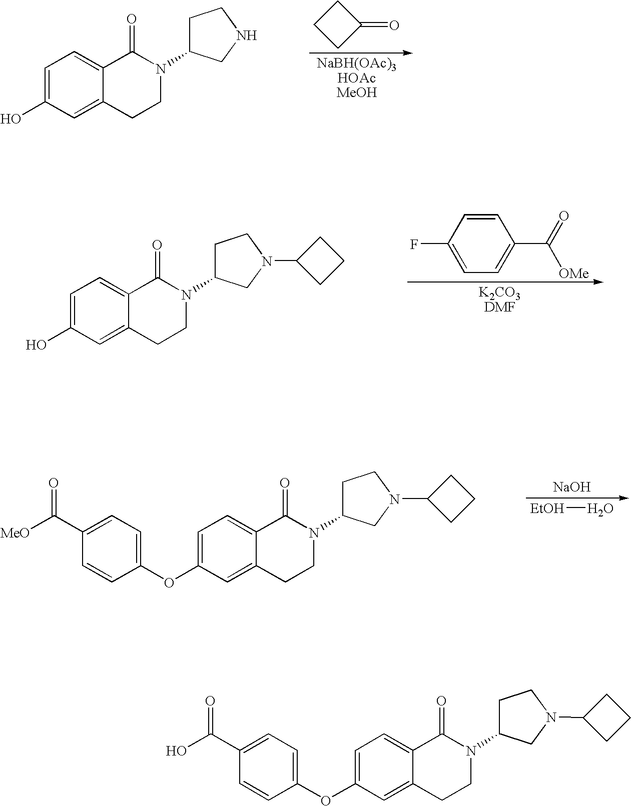

### Step 1: (R)-2-(1-Cyclobutylpyrrolidin-3-yl)-6-hydroxy-3,4-dihydroisoquinolin-1(2H)-one

Using essentially the same procedure described in step 5 of Example 1 and employing cyclobutanone and (R)-6-hydroxy-2-(pyrrolidin-3-yl)-3,4-dihydroisoquinolin-1(2H)-one (1.12 g, 4.8 mmol), the title compound of (R)-2-(1-cyclobutylpyrrolidin-3-yl)-6-hydroxy-3,4-dihydroisoquinolin-1(2H)-one (1.2 g, 87%) was prepared as a clear oil. [α]_{D}²⁵=−29° (c=1% SOLUTION, MeOH); MS (ES) m/z 287.2 [M+H]⁺.

### Step 2: (R)-Methyl 4-(2-(1-cyclobutylpyrrolidin-3-yl)-1-oxo-1,2,3,4-tetrahydroiso-quinolin-6-yloxy)benzoate

Using essentially the same procedure described in step 6 of Example 1 and employing (R)-2-(1-cyclobutylpyrrolidin-3-yl)-6-hydroxy-3,4-dihydroisoquinolin-1(2H)-one (0.7 g, 2.4 mmol), the title compound of (R)-methyl 4-(2-(1-cyclobutylpyrrolidin-3-yl)-1-oxo-1,2,3,4-tetrahydroiso-quinolin-6-yloxy)benzoate (0.53 g, 52%) was formed as a light yellow oil. [α]_{D}²⁵=−14° (c=1% SOLUTION, MeOH); MS (ES) m/z 421.1 [M+H]⁺.

### Step 3: (R)-4-(2-(1-Cyclobutylpyrrolidin-3-yl)-1-oxo-1,2,3,4-tetrahydroiso-quinolin-6-yloxy)benzoic acid

Using essentially the same procedure described in Example 2 and employing (R)-methyl 4-(2-(1-cyclobutylpyrrolidin-3-yl)-1-oxo-1,2,3,4-tetrahydroiso-quinolin-6-yloxy)-benzoate (0.53 g, 1.3 mmol), the title compound of (R)-4-(2-(1-cyclobutylpyrrolidin-3-yl)-1-oxo-1,2,3,4-tetrahydroisoquinolin-6-yloxy)benzoic acid was formed as a white foam, [α]_{D}²⁵=−15.0° (c=1% SOLUTION, MeOH); MS (ES) m/z 405.2 [M+H]⁺.

### Example 33-35

### Preparation of (R)-4-(2-(1-cyclobutylpyrrolidin-3-yl)-1-oxo-1,2,3,4-tetrahydro-isoquinolin-6-yloxy)-benzamide hydrochloride compounds

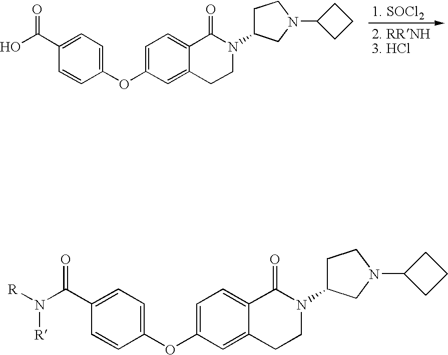

Using essentially the same procedure described in Example 3 and employing the desired amines, the compounds shown in Table IV were obtained and identified by NMR and mass spectral analyses.

TABLE IV

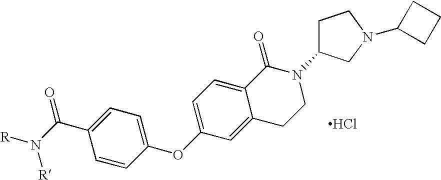

| Ex. No. | R | R′ | mp ° C. | [α]_{D}²⁵ | [M + H] |
|---|---|---|---|---|---|
| 33 | H | methyl | 206-208 | −24 | 420.1 |
| 34 | H | ethyl | 153-154 | −25 | 432.2 |

| | | | | |
|---|---|---|---|---|
| 35 | pyrrolidne | foam | −17 | 460.2 |

### Example 36

### Preparation of (R)-4-(2-(1-cyclopentylpyrrolidin-3-yl)-1-oxo-1,2,3,4-tetrahydro-isoquinolin-6-yloxy)benzoic acid

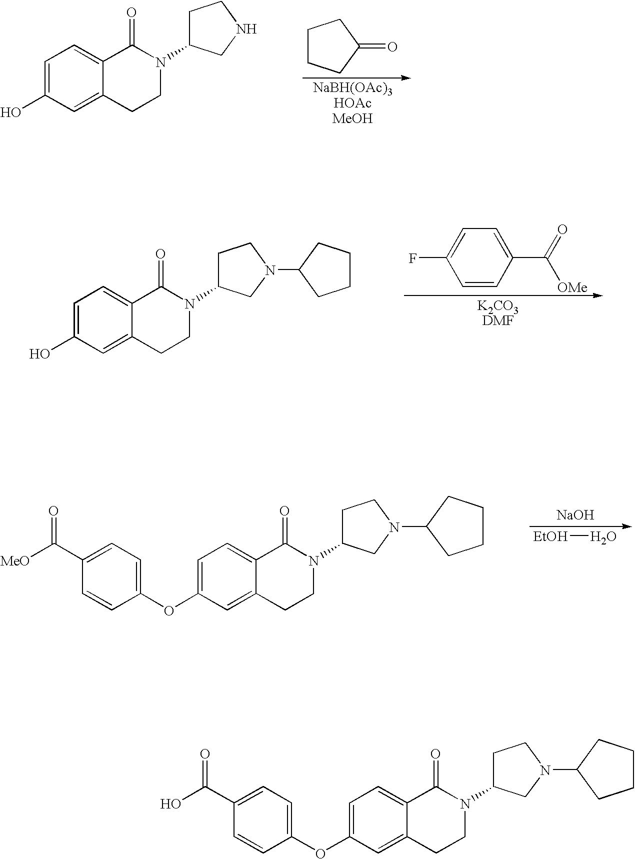

### Step 1: (R)-2-(1-Cyclopentylpyrrolidin-3-yl)-6-hydroxy-3,4-dihydroisoquinolin-1(2H)-one

Using essentially the same procedure described in step 5 of Example 1 and employing the desired cyclopentanone and (R)-6-hydroxy-2-(pyrrolidin-3-yl)-3,4-dihydroisoquinolin-1(2H)-one (0.98 g, 2.3 mmol), the title compound of (R)-2-(1-cyclopentylpyrrolidin-3-yl)-6-hydroxy-3,4-dihydroisoquinolin-1(2H)-one (0.47 g, 67%) was prepared as a clear oil. [α]_{D}²⁵=−26° (c=1% SOLUTION, MeOH); MS (ES) m/z 299.1 [M+H]⁺.

### Step 2: (R)-methyl 4-(2-(1-cyclopentylpyrrolidin-3-yl)-1-oxo-1,2,3,4-tetrahydroiso-quinolin-6-yloxy)benzoate

Using essentially the same procedure described in step 6 of Example 1 and employing (R)-2-(1-cyclopentylpyrrolidin-3-yl)-6-hydroxy-3,4-dihydroisoquinolin-1(2H)-one (0.47 g, 1.6 mmol), the title compound of (R)-methyl 4-(2-(1-cyclopentylpyrrolidin-3-yl)-1-oxo-1,2,3,4-tetrahydroiso-quinolin-6-yloxy)benzoate (0.32 g, 47%) was formed as a light yellow oil. [α]_{D}²⁵=−8° (c=1% SOLUTION, MeOH); MS (ES) m/z 435.1 [M+H]⁺.

### Step 3: (R)-4-(2-(1-Cyclopentylpyrrolidin-3-yl)-1-oxo-1,2,3,4-tetrahydroiso-quinolin-6-yloxy)benzoic acid

Using essentially the same procedure described in Example 2 and employing (R)-methyl 4-(2-(1-cyclopentylpyrrolidin-3-yl)-1-oxo-1,2,3,4-tetrahydroiso-quinolin-6-yloxy)-benzoate (0.29 g, 0.67 mmol), the title compound of (R)-4-(2-(1-cyclopentylpyrrolidin-3-yl)-1-oxo-1,2,3,4-tetrahydroisoquinolin-6-yloxy)benzoic acid (0.21 g) was formed as a white solid, mp 170-173° C., [α]_{D}²⁵=−8.0° (c=1% SOLUTION, MeOH); HRMS: calcd for C₂₅H₂₈N₂O₄+H⁺, 421.21218; found (ESI, [M+H]+ Obs'd), 421.2118.

### Example 37-39

### Preparation of (R)-4-(2-(1-Cyclopentylpyrrolidin-3-yl)-1-oxo-1,2,3,4-tetrahydro-isoquinolin-6-yloxy)-benzamide hydrochloride compounds

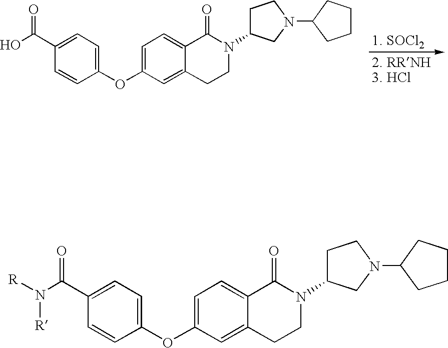

Using essentially the same procedure described in Example 3 and employing the desired amines, the compounds shown in Table V were obtained and identified by NMR and mass spectral analyses.

TABLE V

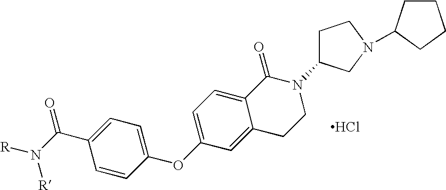

| Ex. No. | R | R′ | mp ° C. | [α]_{D}^{25*} | [M + H] |
|---|---|---|---|---|---|
| 37 | H | methyl | 223-224 | −20 | 434.2 |
| 38 | H | ethyl | 178-180 | −24 | 448.2 |

| | | | | |
|---|---|---|---|---|
| 39 | pyrrolidne | foam | −25 | 474.2 |

^{*}1% solution in MeOH.

### Example 40

### Preparation of (R)-2-(1-cyclopentylpyrrolidin-3-yl)-1-oxo-1,2,3,4-tetrahydroiso-quinolin-6-yl trifluoromethanesulfonate

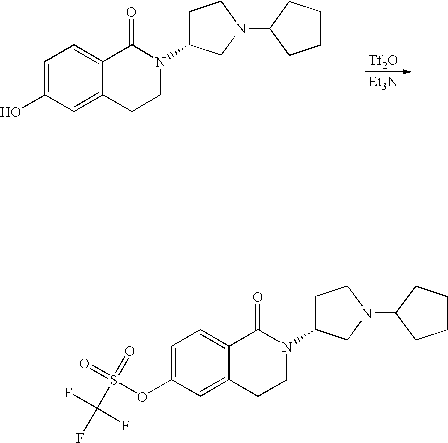

To a solution of (R)-2-(1-cyclopentylpyrrolidin-3-yl)-6-hydroxy-3,4-dihydroisoquinolin-1(2H)-one (0.33 g, 1.1 mmol) in methylene chloride (20 mL) at −15° C. was added trifoluoromethanesulfonic anhydride (0.48 mL, 2.8 mmol) and triethylamine (0.38 mL, 2.7 mmol). The reaction mixture was allowed to stir at −10° C. for 2 h, then partitioned between methylene chloride and aqueous sodium bicarbonate. The aqueous layer was extracted with methylene chloride (3×50 mL), the combined organic layers were dried (sodium sulfate), the solvent was removed in vacuo and the residue was purified by by ISCO CombiFlash® chromatography (silica, 0-10% methanol in dichloromethane plus 0.5% ammonium hydroxide) to afford 0.22 g (46%) of (R)-2-(1-cyclopentylpyrrolidin-3-yl)-1-oxo-1,2,3,4-tetrahydroisoquinolin-6-yl trifluoromethane-sulfonate as a colorless oil. [α]_{D}²⁵=−6° (c=1% SOLUTION, MeOH); MS (ES) m/z 433.1 [M+H]⁺.

### Example 41-43

### Preparation of (R)-4-(2-(1-cyclopentylpyrrolidin-3-yl)-1-oxo-1,2,3,4-tetrahydro-isoquinolin-6-yl)-benzamide hydrochloride compounds

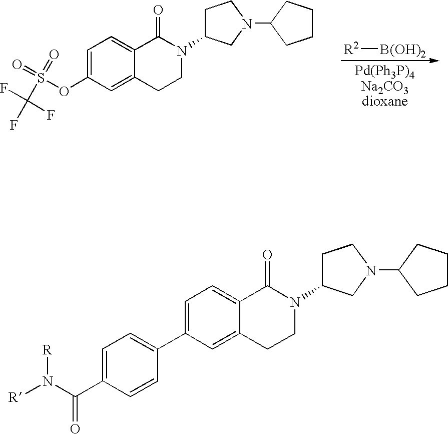

To a solution of (R)-2-(1-cyclopentylpyrrolidin-3-yl)-1-oxo-1,2,3,4-tetrahydroisoquinolin-6-yl trifluoromethanesulfonate (1 eq) and the desired boronic acids (2.0 eq) in dioxane (10 mL) at 90° C. was added tetrakistriphenylphosphine palladium (0) (0.05 eq), sodium carbonate (2.5 eq) and water (2.0 mL). The reaction mixture was heated at 90° C. for 0.5 h, cooled to room temperature and filtered through a pad of celite. The filtrate was diluted with 1N sodium hydroxide and extracted with dichloromethane. The combined extracts were concentrated in vacuo. The residue was purified by ISCO CombiFlash® chromatography (silica gel, 0-10% methanol in dichloromethane with 0.5% ammonium hydroxide). The compounds shown in Table VI were obtained and identified by NMR and mass spectral analyses.

TABLE VI

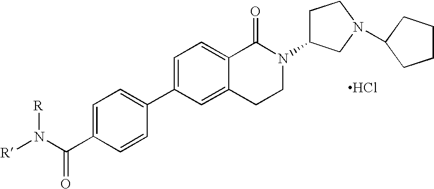

| Ex. No. | R | R′ | mp ° C. | [α]_{D}^{25*} | [M + H] |
|---|---|---|---|---|---|
| 41 | H | methyl | >240 | −24 | 418.2 |
| 42 | H | ethyl | >200 | −33 | 432.2 |

| | | | | |
|---|---|---|---|---|
| 43 | pyrrolidne | >200 | −33 | 458.2 |

1% solution in methaniol

### Example 44-49

### Preparation of (R)-4-(2-(1-cyclobutylpyrrolidin-3-yl)-1-oxo-1,2,3,4-tetrahydro-isoquinolin-6-yl)-benzamide hydrochloride compounds

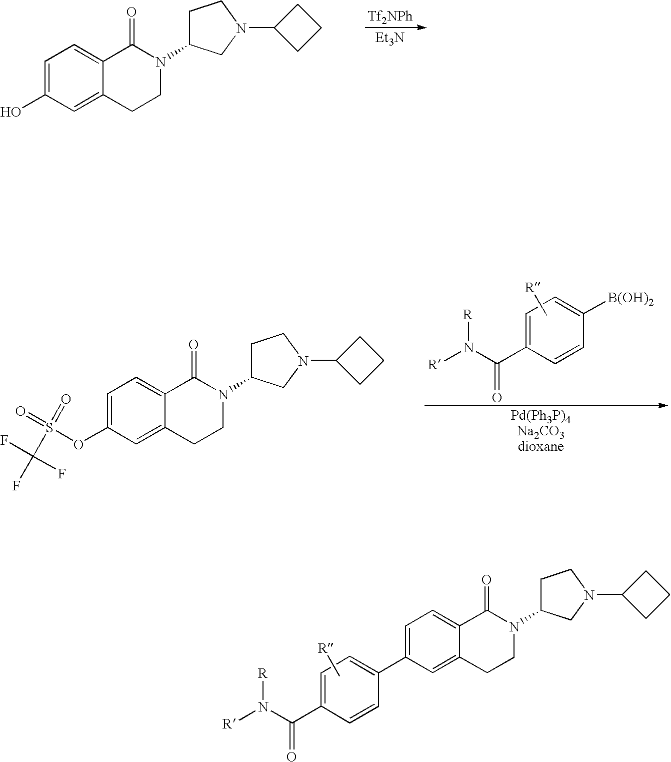

### Step 1: (R)-2-(1-Cyclobutylpyrrolidin-3-yl)-1-oxo-1,2,3,4-tetrahydroisoquinolin-6-yl trifluoromethanesulfonate

To a solution of (R)-2-(1-Cyclobutylpyrrolidin-3-yl)-6-hydroxy-3,4-dihydroisoquinolin-1(2H)-one (0.25 g, 0.87 mmol) in methylene chloride (20 mL) at 0° C. was added N-phenyltrifluoromethanesulfonimide (0.46 g, 1.3 mmol) and triethylamine (0.18 mL, 1.3 mmol). The reaction mixture was allowed to stir at room temperature overnight. The reaction mixture was partitioned between methylene chloride and water. The aqueous layer was extracted with methylene chloride (3×50 mL), the combined organic layers dried (sodium sulfate), the solvent was removed in vacuo and the residue was purified by by ISCO CombiFlash® chromatography (silica, 0-10% methanol in dichloromethane plus 0.5% ammonium hydroxide) to afford 0.30 g (82%) of (R)-2-(1-Cyclobutylpyrrolidin-3-yl)-1-oxo-1,2,3,4-tetrahydroisoquinolin-6-yl trifluoromethanesulfonate as a white foam; [α]_{D}²⁵=−6° (c=1% SOLUTION, MeOH); MS (ES) m/z 419.1 [M+H]⁺.

### Step 2: (R)-4-(2-(1-Cyclobutylpyrrolidin-3-yl)-1-oxo-1,2,3,4-tetrahydro-isoqui-nolin-6-yl)-benzamide hydrochloride compounds

Using essentially the same procedure described in Example 41 and employing the desired boronic acids and (R)-2-(1-cyclobutylpyrrolidin-3-yl)-1-oxo-1,2,3,4-tetrahydroisoquinolin-6-yl trifluoromethanesulfonate, the compounds shown in Table VII were obtained and identified by NMR and mass spectral analyses.

TABLE VII

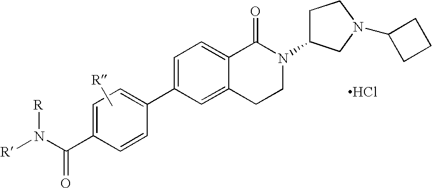

| Ex. No. | R | R′ | R″ | mp ° C. | [α]_{D}^{25*} | [M + H] |
|---|---|---|---|---|---|---|
| 44 | H | methyl | H | 263-265 | −35 | 404.2333 |
| 45 | H | ethyl | H | 268-272 | −33 | 418.2492 |
| 46 | Me | Me | H | 211-212 | −33 | 418.2492 |

| | | | | | |
|---|---|---|---|---|---|
| 47 | pyrrolidne | H | 215-217 | −23 | 444.2640 |
| 48 | pyrrolidne | 3-F | 235-236 | −25 | 462.2551 |
| 49 | pyrrolidne | 3-Cl | 198-200 | −24 | 478.2258 |

^{*}1% solution in methaniol

### Example 50-51

### Preparation of (R)-2-(1-cyclobutylpyrrolidin-3-yl)-6-(substituted phenyl)-3,4-dihydro-isoquinolin-1(2H)-one hydrochlorides

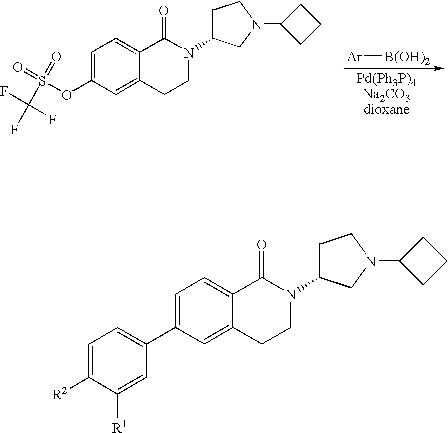

Using essentially the same procedure described in Example 41 and employing the desired boronic acids and (R)-2-(1-cyclobutylpyrrolidin-3-yl)-1-oxo-1,2,3,4-tetrahydroisoquinolin-6-yl trifluoromethanesulfonate, the compounds shown in Table VIII were obtained and identified by NMR and high resolution mass spectral analyses.

TABLE VIII

| Ex. | | | | | |
|---|---|---|---|---|---|
| No. | R¹ | R² | mp ° C. | [α]_{D}^{25*} | [M + H] |
| 50 | H | H | >255° C. | −36 | 347.2122 |
| 51 | H | 4-F | 231-233 | −26. | 365.2027 |
| 52 | CONHMe | H | 203-205 | −26 | 444.2649 |
| 53 | CONHMe | F | foam | −22 | 462.2561 |

*1% solution in methanol

### Example 54

### Preparation of (R)-2-(pyrrolidin-3-yl)-6-(4-(pyrrolidine-1-carbonyl)phenyl)-3,4-dihydro-isoquinolin-1(2H)-one

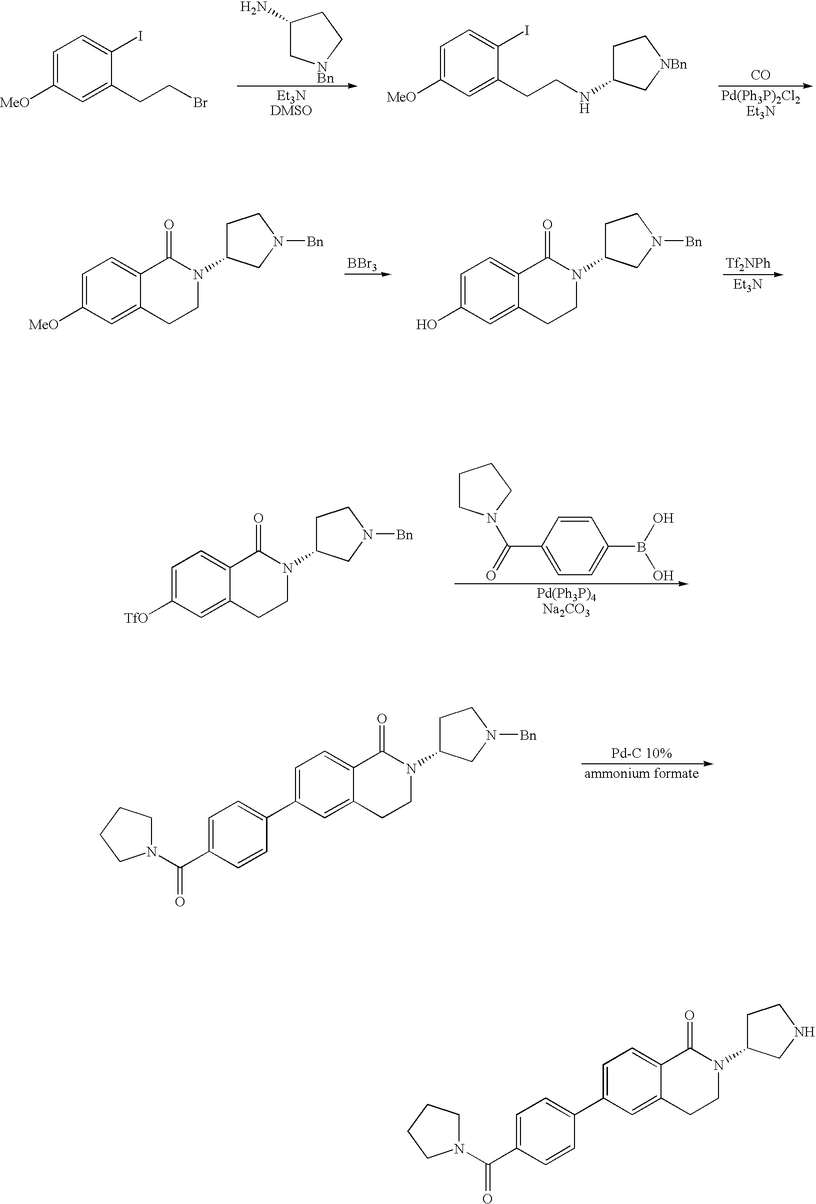

### Step 1: (R)-1-Benzyl-N-(2-iodo-5-methoxyphenethyl)pyrrolidin-3-amine

Using essentially the same procedure described in Step 2 of Example 1 and employing the (R)-1-benzylpyrrolidin-3-amine (2.3 g, 13.2 mmol), the title product of (R)-1-benzyl-N-(2-iodo-5-methoxyphenethyl)pyrrolidin-3-amine (1.34 g, 35%) was obtained as a light brown oil, [α]_{D}²⁵=−3° MS (ES) m/z 437.0 [M+H]⁺.

### Step 2: (R)-2-(1-Benzylpyrrolidin-3-yl)-6-methoxy-3,4-dihydroisoquinolin-1(2H)-one

Using essentially the same procedure described in Step 3 of Example 1 and employing (R)-1-benzyl-N-(2-iodo-5-methoxyphenethyl)pyrrolidin-3-amine (1.34 g, 3.1 mmol), the title product of (R)-2-(1-benzylpyrrolidin-3-yl)-6-methoxy-3,4-dihydroisoquinolin-1(2H)-one (0.68 g, 66%) was obtained as a light brown oil, [α]_{D}²⁵=−13° c=1% SOLUTION, MeOH);MS (ES) m/z 337.2 [M+H]⁺.

### Step 3: (R)-2-(1-Benzylpyrrolidin-3-yl)-6-hydroxy-3,4-dihydroisoquinolin-1(2H)-one

Using essentially the same procedure described in Step 4 of Example 1 and employing (R)-2-(1-benzylpyrrolidin-3-yl)-6-methoxy-3,4-dihydroisoquinolin-1(2H)-one (2.72 g, 8.1 mmol), the title product of (R)-2-(1-benzylpyrrolidin-3-yl)-6-hydroxy-3,4-dihydroisoquinolin-1(2H)-one (2.17 g, 83%) can be obtained as a white foam, [α]_{D}²⁵=−12° (c=1% SOLUTION, MeOH); HRMS (ES) m/z 323.1758 [M+H]⁺.

### Step 4: (R)-2-(1-Benzylpyrrolidin-3-yl)-1-oxo-1,2,3,4-tetrahydroisoquinolin-6-yl trifluoromethanesulfonate

To a solution of (R)-2-(1-benzylpyrrolidin-3-yl)-6-hydroxy-3,4-dihydroisoquinolin-1(2H)-one (0.11 g, 0.34 mmol) in methylene chloride (20 mL) at 0° C. was added N-phenyltrifluoromethanesulfonimide (0.18 g, 0.5 mmol) and triethylamine (0.07 mL, 0.5 mmol). The reaction mixture was allowed to stir at room temperature overnight. The reaction mixture was partitioned between methylene chloride and water. The aqueous layer was extracted with methylene chloride (3×50 mL), the combined organic layers were dried (sodium sulfate), the solvent was removed in vacuo and the residue was purified by by ISCO CombiFlash® chromatography (silica, 0-10% methanol in dichloromethane plus 0.5% ammonium hydroxide) to afford 0.13 g (84%) of (R)-2-(1-benzylpyrrolidin-3-yl)-1-oxo-1,2,3,4-tetrahydroisoquinolin-6-yl trifluoromethane-sulfonateas a colorless oil. [α]_{D}²⁵=−4° (c=1% SOLUTION, MeOH); MS (ES) m/z 455.1 [M+H]⁺.

### Step 5: (R)-2-(1-Benzylpyrrolidin-3-yl)-6-(4-(pyrrolidine-1-carbonyl)phenyl)-3,4-dihydroisoquinolin-1(2H)-one

Using essentially the same procedure described in Example 41 and employing 4-(pyrrolidine-1-carbonyl)phenylboronic acid (1.2 g, 5.4 mmol) and (R)-2-(1-benzyl-pyrrolidin-3-yl)-1-oxo-1,2,3,4-tetrahydroisoquinolin-6-yl trifluoromethane-sulfonate (1.3 g, 2.7 mmol), the desired product of (R)-2-(1-benzylpyrrolidin-3-yl)-6-(4-(pyrrolidine-1-carbonyl)phenyl)-3,4-dihydroisoquinolin-1(2H)-one (1.2 g, 92%) was obtained as light yellow foam. MS (ES) m/z [M+H]⁺. mp 255-256° C.; MS (ESI) m/z 480.2;

HRMS: calcd for C₃₁H₃₃N₃O₂+H⁺, 480.26455; found (ESI, [M+H]+ Obs'd), 480.2644.

### Step 6: (R)-2-(Pyrrolidin-3-yl)-6-(4-(pyrrolidine-1-carbonyl)phenyl)-3,4-dihydro-isoquinolin-1(2H)-one

A solution of (R)-2-(1-benzylpyrrolidin-3-yl)-6-(4-(pyrrolidine-1-carbonyl)phenyl)-3,4-dihydroisoquinolin-1(2H)-one (1.05 g, 2.2 mmol) in ethanol at room temperature was treated with Pd—C 10% (0.10 g) and ammonium formate (0.69 g, 11 mmol). The reaction mixture was heated at 85° C. for 3 h, cooled to room temperature and filtered. The filtercake was washed with ethanol and the combined filtrates were concentrated in vacuo. The residue was purified by ISCO CombiFlash® chromatography (silica, 0-10% methanol/dichloromethane plus 0.5% ammonium hydroxide) to provide the title compound 0.56 g (66%) as a white foam, [α]_{D}²⁵=−15° (c=1% SOLUTION, MeOH); HRMS: calcd for C₂₄H₂₇N₃O₂+H⁺, 390.21760; found (ESI, [M+H]+ Calc'd), 390.2176;

### Example 55-62

### Preparation of (R)-4-(2-(1-substituted-pyrrolidin-3-yl)-1-oxo-1,2,3,4-tetrahydro-isoquinolin-6-yl)-benzamide hydrochloride compounds

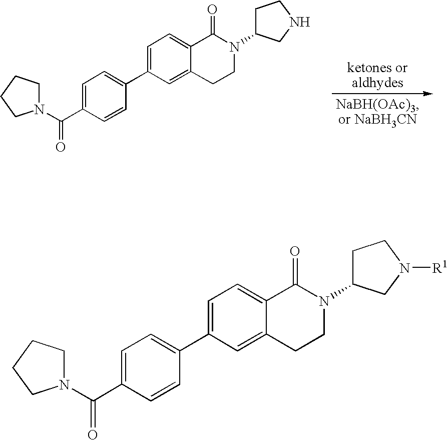

Using essentially the same procedure described in Example 23 and employing the desired aldehyde or ketone, the compounds shown in Table IX were obtained and identified by NMR and high resolution mass spectral analyses.

TABLE IX

| Ex. | | | | |
|---|---|---|---|---|
| No. | R¹ | mp ° C. | [α]_{D}^{25*} | [M + H] |
| 55 | Me | >230 | −45 | 404.2344 |
| 56 | i-pr | 220-222 | −19 | 432.2647 |
| 57 | cyclohexyl | 279-281 | −35 | 472.2961 |
| 58 | cyclopropyl methyl | 287-288 | −47 | 444.2647 |
| 59 | cyclopentylmethyl | 288-290 | −54 | 472.2961 |
| 60 | furan-3-ylmethyl | 254-256 | −62 | 470.2441 |
| 61 | furan-2-ylmethyl | 254-256 | −57 | 470.2438 |
| 62 | thiophen-2-ylmethyl | 249-250 | −69 | 486.2212 |

*1% solution in methanol

### Example 63

### Preparation of 2-(1-benzylpiperidin-4-yl)-6-(4-(pyrrolidine-1-carbonyl)phenyl)-3,4-dihydroisoquinolin-1(2H)-one

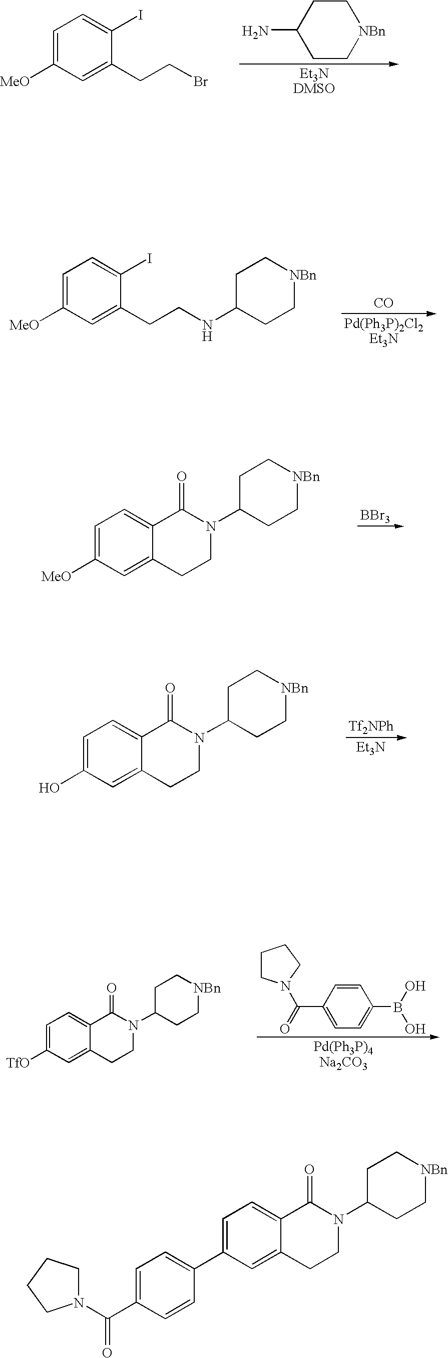

### Step 1: 1-Benzyl-N-(2-iodo-5-methoxyphenethyl)piperidin-4-amine

Using essentially the same procedure described in Step 2 of Example 1 and employing the 1-benzylpiperidin-4-amine (7.2 mL, 0.035 mol), the title product of 1-benzyl-N-(2-iodo-5-methoxyphenethyl)piperidin-4-amine (2.3 g, 29%) was obtained as a light brown oil, MS (ES) m/z 451.1 [M+H]⁺.

### Step 2: 2-(1-benzylpiperidin-4-yl)-6-methoxy-3,4-dihydroisoquinolin-1(2H)-one

Using essentially the same procedure described in Step 3 of Example 1 and employing 1-benzyl-N-(2-iodo-5-methoxyphenethyl)piperidin-4-amine (2.25 g, 5.0 mmol), the title product of 2-(1-benzylpiperidin-4-yl)-6-methoxy-3,4-dihydroisoquinolin-1(2H)-one (0.7 g, 40%) was obtained as a light brown oil, MS (ES) m/z 351.2 [M+H]⁺.

### Step 3: 2-(1-benzylpiperidin-4-yl)-6-hydroxy-3,4-dihydroisoquinolin-1(2H)-one

Using essentially the same procedure described in Step 4 of Example 1 and employing 2-{1-benzylpiperidin-4-yl)-6-methoxy-3,4-dihydroisoquinolin-1(2H)-one (0.7 g, 2.0 mmol), the title product of 2-(1-benzylpiperidin-4-yl)-6-hydroxy-3,4-dihydroisoquinolin-1(2H)-one (0.34 g, 51%) can be obtained as a white foam, MS (ES) m/z 337.2 [M+H]⁺.

### Step 4: 2-(1-benzylpiperidin-4-yl)-1-oxo-1,2,3,4-tetrahydroisoquinolin-6-yl trifluoromethanesulfonate

Using essentially the same procedure described in Step 4 of Example 54 and employing 2-(1-benzylpiperidin-4-yl)-6-hydroxy-3,4-dihydroisoquinolin-1(2H)-one (0.34 g, 1.0 mmol), the title product of 2-(1-benzylpiperidin-4-yl)-1-oxo-1,2,3,4-tetrahydroisoquinolin-6-yl trifluoromethanesulfonate (0.52 g, 100%) can be obtaianed as a white foam, MS (ES) m/z 469.1 [M+H]⁺.

### Step 5: 2-(1-benzylpiperidin-4-yl)-6-(4-(pyrrolidine-1-carbonyl)phenyl)-3,4-dihydroisoquinolin-1(2H)-one

Using essentially the same procedure described in Step 5 of Example 54 and employing 2-(1-benzylpiperidin-4-yl)-1-oxo-1,2,3,4-tetrahydroisoquinolin-6-yl trifluoro-methanesulfonate (0.52 g, 1.1 mmol), the title product of 2-(1-benzylpiperidin-4-yl)-6-(4-(pyrrolidine-1-carbonyl)phenyl)-3,4-dihydroisoquinolin-1(2H)-one (0.54 g, 98%) can be obtaianed as a white solid, mp 197-198° C., MS (ES) m/z 494.2 [M+H]⁺.

### Example 64

### Preparation of 2-(piperidin-4-yl)-6-(4-(pyrrolidine-1-carbonyl)phenyl)-3,4-dihydroisoquinolin-1(2H)-one

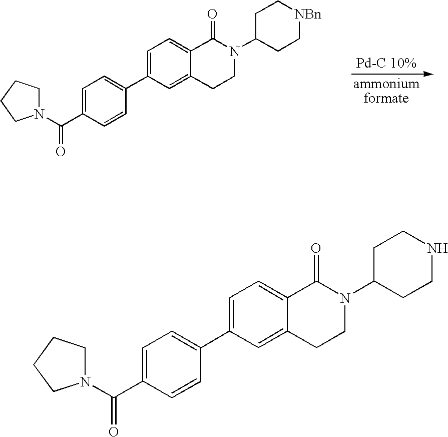

Using essentially the same procedure described in step 6 of Example 54 and employing 2-(1-benzylpiperidin-4-yl)-6-(4-(pyrrolidine-1-carbonyl)phenyl)-3,4-dihydroisoquinolin-1(2H)-one (0.53 g, 2.1 mmol), the title product of 2-(piperidin-4-yl)-6-(4-(pyrrolidine-1-carbonyl)phenyl)-3,4-dihydroisoquinolin-1(2H)-one (0.3 g, 69%) was obtained as a white solid, mp>273° C., HRMS: calcd for C₂₅H₂₉N₃O₂+H⁺, 404.23325; found (ESI, [M+H]+ Obs'd), 404.2335.

### Example 65-67

### Preparation of 2-(1-substituted piperidin-4-yl)-6-(4-(pyrrolidine-1-carbonyl)-phenyl)-3,4-dihydroisoquinolin-1(2H)-one hydrochloride compounds

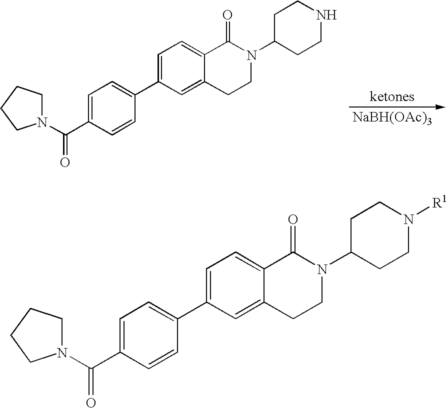

Using essentially the same procedure described in Example 23 and employing the desired aldehyde or ketone, the compounds shown in Table X were obtained and identified by NMR and high resolution mass spectral analyses.

TABLE X

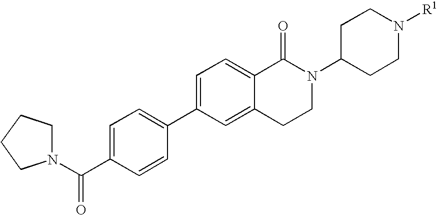

| Ex. No. | R¹ | mp ° C. | [M + H] |
|---|---|---|---|
| 65 | cyclobutyl | >300 | 458.2806 |
| 66 | cyclopentyl | >300 | 472.2963 |
| 67 | isopropyl | >300 | 446.2808 |

### Example 68

### Preparation of (R)-2-(1-benzylpiperidin-3-yl)-6-(4-(pyrrolidine-1-carbonyl)-phenyl)-3,4-dihydroisoquinolin-1(2H)-one

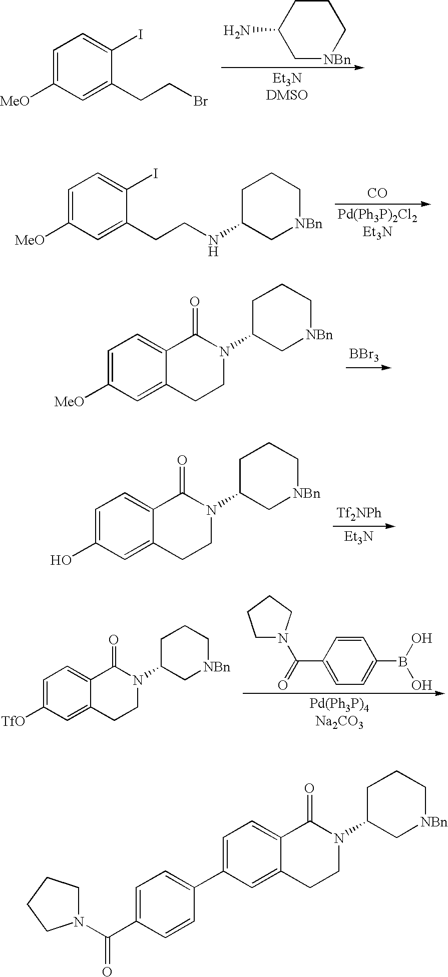

### Step 1: (R)-1-Benzyl-N-(2-iodo-5-methoxyphenethyl)piperidin-3-amine

Using essentially the same procedure described in Step 2 of Example 1 and employing the (R)-1-benzylpiperidin-3-amine (6.4 g, 33 mmol), the title product of (R)-1-benzyl-N-(2-iodo-5-methoxyphenethyl)piperidin-3-amine (3.9 g, 39%) was obtained as a light brown oil, [α]_{D}²⁵=−5° (c=1% SOLUTION, MeOH); HRMS (ES) m/z 451.1241 [M+H]⁺.

### Step 2: (R)-2-(1-Benzylpiperidin-3-yl)-6-methoxy-3,4-dihydroisoquinolin-1(2H)-one

Using essentially the same procedure described in Step 3 of Example 1 and employing (R)-1-benzyl-N-(2-iodo-5-methoxyphenethyl)piperidin-3-amine (3.89 g, 8.6 mmol), the title product of (R)-2-(1-benzylpiperidin-3-yl)-6-methoxy-3,4-dihydroisoquinolin-1(2H)-one (2.3 g, 75%) was obtained as a light brown oil, [α]_{D}²⁵=−13° (c=1% SOLUTION, MeOH); HRMS (ES) m/z 351.2067 [M+H]⁺.

### Step 3: (R)-2-(1-Benzylpiperidin-3-yl)-6-hydroxy-3,4-dihydroisoquinolin-1(2H)-one

Using essentially the same procedure described in Step 4 of Example 1 and employing (R)-2-(1-benzylpiperidin-3-yl)-6-methoxy-3,4-dihydroisoquinolin-1(2H)-one (2.26 g, 6.4 mmol), the title product 0.56 g (26%) was obtained as a white foam, [α]_{D}²⁵=−15° (c=1% SOLUTION, MeOH); MS (ES) m/z 337.2 [M+H]⁺.

### Step 4: (R)-2-(1-Benzylpiperidin-3-yl)-1-oxo-1,2,3,4-tetrahydroisoquinolin-6-yl trifluoromethanesulfonate

Using essentially the same procedure described in Step 4 of Example 54 and employing (R)-2-(1-benzylpiperidin-3-yl)-6-hydroxy-3,4-dihydroisoquinolin-1(2H)-one (0.57 g, 1.7 mmol), the title product of (R)-2-(1-benzylpiperidin-3-yl)-1-oxo-1,2,3,4-tetrahydroisoquinolin-6-yl trifluoromethanesulfonate (0.77 g, 97%) was obtained as a white foam, [α]_{D}²⁵=−15° (c=1% SOLUTION, MeOH); HRMS (ES) m/z 469.1403 [M+H]⁺.

### Step 5: (R)-2-(1-Benzylpiperidin-3-yl)-6-(4-(pyrrolidine-1-carbonyl)phenyl)-3,4-dihydroisoquinolin-1(2H)-one

Using essentially the same procedure described in Step 5 of Example 54 and employing (R)-2-(1-benzylpiperidin-3-yl)-1-oxo-1,2,3,4-tetrahydroisoquinolin-6-yl trifluoromethanesulfonate (0.77 g, 1.6 mmol), the title product of (R)-2-(1-benzylpiperidin-3-yl)-6-(4-(pyrrolidine-1-carbonyl)phenyl)-3,4-dihydroisoquinolin-1(2H)-one (0.58 g, 72%) was obtained as a white foam, [α]_{D}²⁵=−33° (c=1% SOLUTION, MeOH); H RMS (ES) m/z 494.2804 [M+H]⁺.

### Example 69

### Preparation of (R)-2-(piperidin-3-yl)-6-(4-(pyrrolidine-1-carbonyl)phenyl)-3,4-dihydroiso-quinolin-1(2H)-one

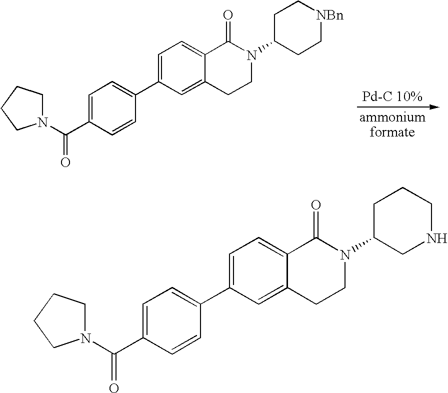

Using essentially the same procedure described in step 6 of Example 54 and employing (R)-2-(1-benzylpiperidin-3-yl)-6-(4-(pyrrolidine-1-carbonyl)phenyl)-3,4-dihydroisoquinolin-1(2H)-one (0.56 g, 1.1 mmol), the title product of (R)-2-(piperidin-3-yl)-6-(4-(pyrrolidine-1-carbonyl)phenyl)-3,4-dihydroiso-quinolin-1(2H)-one (0.38 g, 83%) was obtained as a light brown oil, [α]_{D}²⁵=9° C. (1% SOLUTION, MeOH); HRMS (ES) m/z 404.2337 [M+H]⁺.

### Example 70-72

### Preparation of (R)-2-(1-substituted piperidin-3-yl)-6-(4-(pyrrolidine-1-carbonyl)-phenyl)-3,4-dihydroisoquinolin-1(2H)-one hydrochloride compounds

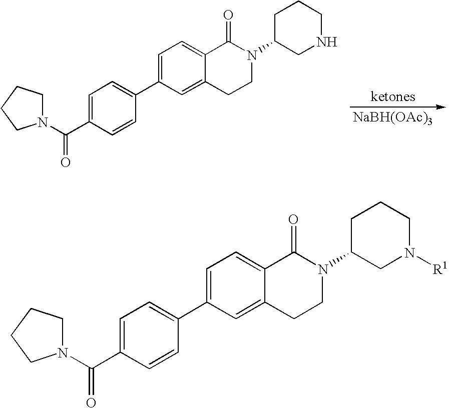

Using essentially the same procedure described in Example 23 and employing the desired aldehyde or ketone, the compounds shown in Table XI were obtained and identified by NMR and high resolution mass spectral analyses.

TABLE XI

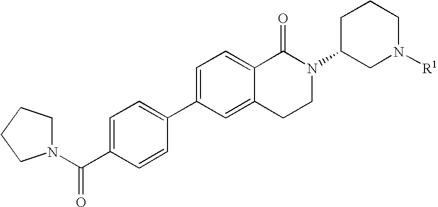

| Ex. No. | R¹ | mp ° C. | [α]_{D}^{25*} | [M + H] |
|---|---|---|---|---|
| 70 | cyclobutyl | 260.5-262 | −16 | 458.2799 |
| 71 | cyclopentyl | >250 | −8 | 472.2965 |
| 72 | isopropyl | 274-275 | 9 | 446.2803 |

^{*}1% solution in methanol

### Example 73-81

### Preparation of 4-{2-[(3R)-1-cyclobutylpyrrolidin-3-yl]-1-oxo-1,2,3,4-tetrahydro-isoquinolin-6-yl}-N-substituted-3-fluorobenzamide hydrochloride compounds

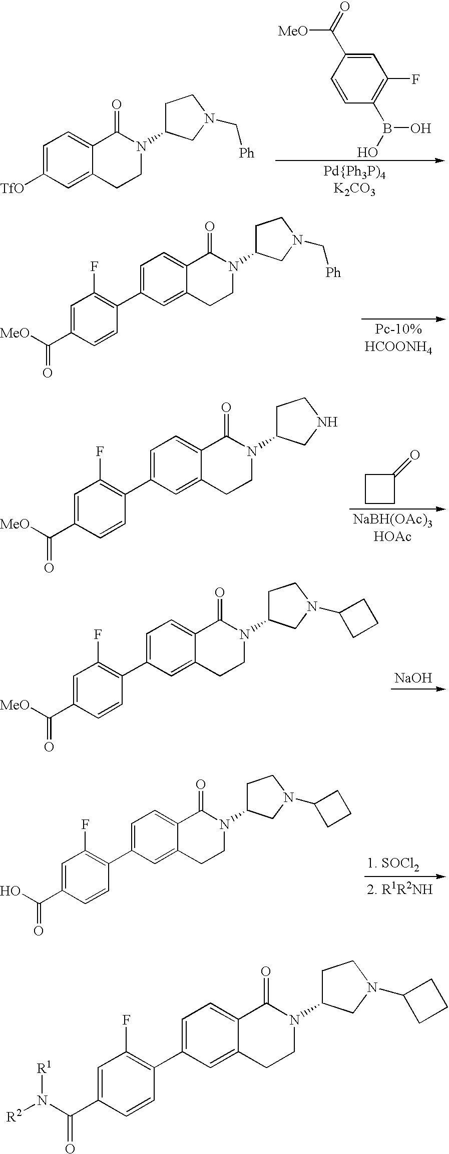

### Step 1: (R)-Methyl 4-(2-(1-benzylpyrrolidin-3-yl)-1-oxo-1,2,3,4-tetrahydroisoqui-nolin-6-yl)-3-fluorobenzoate

Using essentially the same procedure described in Example 41 and employing the desired 2-fluoro-4-(methoxycarbonyl)phenylboronic acid (1.41 g, 6.4 mmol) and (R)-2-(1-benzylpyrrolidin-3-yl)-1-oxo-1,2,3,4-tetrahydroisoquinolin-6-yl trifluoromethane-sulfonate (1.67 g, 3.6 mmol), the title compound 1.6 g (98%) was obtained as a light oil, [α]_{D}²⁵=−24° (1% SOLUTION, MeOH); HRMS (ES) m/z 459.2084 [M+H]⁺.

### Step 2: (R)-Methyl 3-fluoro-4-(1-oxo-2-(pyrrolidin-3-yl)-1,2,3,4-tetrahydroisoquinolin-6-yl)benzoate

Using essentially the same procedure described in step 6 of Example 55 and employing (R)-methyl 4-(2-(1-benzylpyrrolidin-3-yl)-1-oxo-1,2,3,4-tetrahydroisoqui-nolin-6-yl)-3-fluorobenzoate (1.6 g, 3.5 mmol), the title product (0.59 g, 46%) was obtained as a clear oil, [α]_{D}²⁵=−22° (1% SOLUTION, MeOH); HRMS (ES) m/z 369.1611 [M+H]⁺.

### Step 3: (R)-Methyl 4-(2-(1-cyclobutylpyrrolidin-3-yl)-1-oxo-1,2,3,4-tetrahydroiso-quinolin-6-yl)-3-fluorobenzoate

Using essentially the same procedure described in Example 23 and employing cyclobutanone (1.2 mL, 16 mmol) and (R)-methyl 3-fluoro-4-(1-oxo-2-(pyrrolidin-3-yl)-1,2,3,4-tetrahydroisoquinolin-6-yl)benzoate (0.58 g, 1.6 mmol), the title compound 0.47 g (71%) was obtained as a white solid, mp 75-75° C., [α]_{D}²⁵=−21° (1% SOLUTION, MeOH); HRMS (ES) m/z 423.2085 [M+H]⁺.

### Step 4: (R)-4-(2-(1-Cyclobutylpyrrolidin-3-yl)-1-oxo-1,2,3,4-tetrahydroiso-quinolin-6-yl)-3-fluorobenzoic acid

Using essentially the same procedure described in Example 2 and employing (R)-methyl 4-(2-(1-cyclobutylpyrrolidin-3-yl)-1-oxo-1,2,3,4-tetrahydroisoquinolin-6-yl)-3-fluorobenzoate (0.47 g, 1.1 mmol), the title compound 0.38 g (85%) was formed as a white solid, [α]_{D}²⁵=−10° (c=1% SOLUTION, MeOH); HRMS (ES) m/z 409.1922 [M+H]⁺.

### Step 5: (R)-4-(2-(1-Cyclobutylpyrrolidin-3-yl)-1-oxo-1,2,3,4-tetrahydroiso-quinolin-6-yl)-3-fluoro-N-substituted benzamide chlorides

Using essentially the same procedure described in Example 3 and employing the desired amines, the compounds shown in Table XII were obtained and identified by NMR and high resolution mass spectral analyses.

TABLE XII

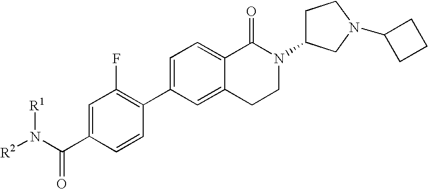

| Ex. No. | R¹R²NH | mp ° C. | [α]_{D}²⁵* | [M + H] |
|---|---|---|---|---|
| 73 | (S)-2-Methylpyrrolidine | 212-213 | 17 | 476.2707 |
| 74 | cyclobutylamine | >230 | — | 462.2550 |
| 75 | methylamine | 220-221 | −23 | 422.1 |
| 76 | (R)-2-Methylpyrrolidine | 239-241 | — | 476.2704 |
| 77 | ethylamine | >205 | −26 | 436.2395 |
| 78 | dimethylamine | 209-210 | −27 | 436.2393 |
| 79 | pyrrolidine | 211-212 | −24 | 462.2552 |
| 80 | cyclopentylamine | foam | — | 476.2706 |
| 81 | Cyclopropylamine | >200 | −24 | 448.2394 |

*1% solution in methaniol

### Example 82-83

### Preparation of (R)-4-(2-(1-isopropylpyrrolidin-3-yl)-1-oxo-1,2,3,4-tetrahydroiso-quinolin-6-yl)-N-substituted benzamide hydrochloride compounds

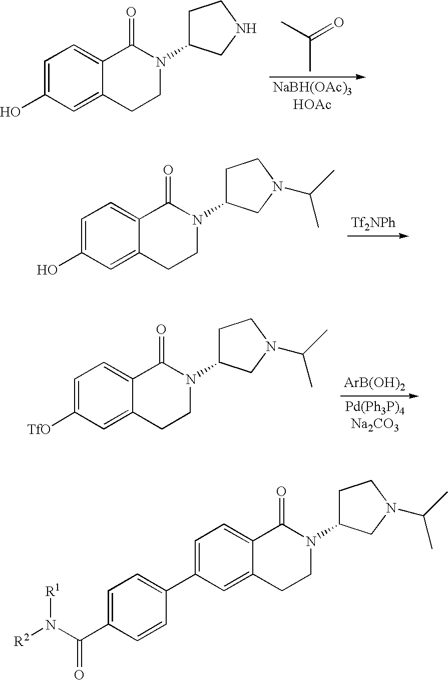

### Step 1: (R)-6-Hydroxy-2-(1-isopropylpyrrolidin-3-yl)-3,4-dihydroisoquinolin-1(2H)-one

Using essentially the same procedure described in Example 23 and employing acetone (2.4 g, 43 mmol) and (R)-6-hydroxy-2-(pyrrolidin-3-yl)-3,4-dihydroisoquinolin-1(2H)-one (1.0 g, 4.3 mmol), the title compound 0.89 g (100%) was obtained as a yellow oil, [α]_{D}²⁵=−14° (1% SOLUTION, MeOH); HRMS (ES) m/z 275.1758 [M+H]⁺.

### Step 2: (R)-2-(1-Isopropylpyrrolidin-3-yl)-1-oxo-1,2,3,4-tetrahydroisoquinolin-6-yl trifluoromethanesulfonate

Using essentially the same procedure described in Step 4 of Example 54 and employing (R)-6-hydroxy-2-(1-isopropylpyrrolidin-3-yl)-3,4-dihydroisoquinolin-1(2H)-one (0.88 g, 3.2 mmol), the title product (1.1 g, 87%) was obtained as a yellow foam, [α]_{D}²⁵=−7° (c=1% SOLUTION, MeOH); HRMS (ES) m/z 407.1251 [M+H]⁺.

### Step 3: (R)-4-(2-(1-Isopropylpyrrolidin-3-yl)-1-oxo-1,2,3,4-tetrahydroisoquinolin-6-yl)-N-substituted benzamide hydrochlorides

Using essentially the same procedure described in Example 41 and employing the desired boronic acids and (R)-2-(1-isopropylpyrrolidin-3-yl)-1-oxo-1,2,3,4-tetrahydroisoquinolin-6-yl trifluoromethanesulfonate, the compounds shown in Table XIV were obtained and identified by NMR and high resolution mass spectral analyses.

TABLE XIV

| Ex. No. | R¹R²NH | mp ° C. | [α]_{D}²⁵* | [M + H] |
|---|---|---|---|---|
| 82 | methylamine | >275 | −35 | 392.2331 |
| 83 | ethylamine | >270 | −36 | 406.2488 |

*1% solution in methanol

### Example 84-86

### Preparation of (R)-3-fluoro-4-(2-(1-isopropylpyrrolidin-3-yl)-1-oxo-1,2,3,4-tetrahydroiso-quinolin-6-yl)-N-methylbenzamide hydrochloride compounds

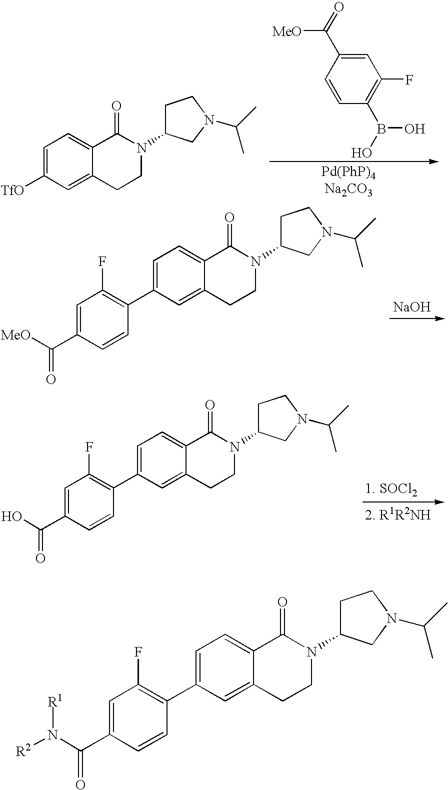

### Step 1: (R)-Methyl 3-fluoro-4-(2-(1-isopropylpyrrolidin-3-yl)-1-oxo-1,2,3,4-tetrahydroisoquinolin-6-yl)benzoate

Using essentially the same procedure described in Example 41 and employing the desired 2-fluoro-4-(methoxycarbonyl)phenylboronic acid (0.36 g, 1.8 mmol) and (R)—(R)-2-(1-isopropylpyrrolidin-3-yl)-1-oxo-1,2,3,4-tetrahydroisoquinolin-6-yl trifluoro-methanesulfonate (0.36 g, 0.89 mmol), the title compound 0.31 g (85%) was obtained as a light oil, [α]_{D}²⁵=−4.8° (1% SOLUTION, MeOH); HRMS (ES) m/z 411.2079 [M+H]⁺.

### Step 2: (R)-3-Fluoro-4-(2-(1-isopropylpyrrolidin-3-yl)-1-oxo-1,2,3,4-tetrahydro-isoquinolin-6-yl)benzoic acid

Using essentially the same procedure described in Example 2 and (R)-methyl 3-fluoro-4-(2-(1-isopropylpyrrolidin-3-yl)-1-oxo-1,2,3,4-tetrahydroisoquinolin-6-yl)benzoate (0.36 g, 0.88 mmol), the title compound 0.31 g (90%) was formed as an off-white foam; [α]_{D}²⁵=−1.6° (c=1% SOLUTION, MeOH); HRMS (ES) m/z 397.1930 [M+H]⁺.

### Step 3: (R)-3-Fluoro-4-(2-(1-isopropylpyrrolidin-3-yl)-1-oxo-1,2,3,4-tetrahydro-isoquinolin-6-yl)-N-substituted benzamide

Using essentially the same procedure described in Example 3 and employing the desired amines, the compounds shown in Table XV were obtained and identified by NMR and high resolution mass spectral analyses.

TABLE XV

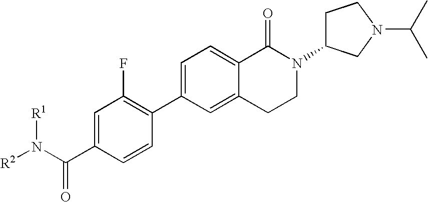

| Ex. No. | R¹R²NH | mp ° C. | [α]_{D}²⁵* | [M + H] |
|---|---|---|---|---|
| 84 | pyrrolidine | 249-250 | −26 | 450.2563 |
| 85 | methylamine | 245-247 | −26 | 410.2244 |
| 86 | ethylamine | >255 | — | 424.2401 |

*1% solution in methaniol

### Example 87

### Preparation of 2-[(3R)-1-cyclobutylpyrrolidin-3-yl]-6-(pyrrolidin-1-ylcarbonyl)-3,4-dihydroisoquinolin-1(2H)-one hydrochloride

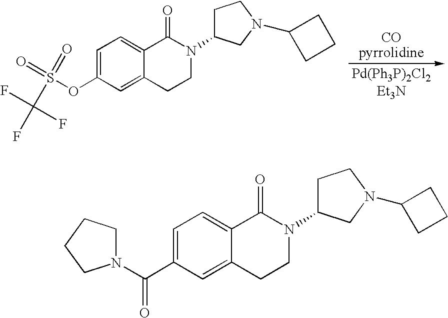

A mixture of (R)-2-(1-cyclobutylpyrrolidin-3-yl)-1-oxo-1,2,3,4-tetrahydroisoquinolin-6-yl trifluoromethanesulfonate (0.09 g, 0.2 mmol), pyrrolidine (0.18 mL, 2.0 mmol), dichlorobis(tri-phenyl-phosphine)palladium (II) (0.15, 0.01 mmol), triethylamine (0.075 mL, 0.5 mmol) in DMF was purged with carbon monoxide for 20 minutes, heated in a sealed tube to 90° C. for 16 h, cooled to room temperature and filtered through a pad of celite. The filtrate was diluted with water and extracted with CH₂Cl₂. The combined extracts were washed with brine, dried over Na₂SO₄ and concentrated in vacuo. The residue was purified by ISCO CombiFlash® chromatography (silica, 0-10% methanol in CH₂Cl₂ with 0.5% ammonium hydroxide) to afford the free amine of the title product as a colorless oil. The oil was dissolved in ethanol, treated with etheral HCl, stirred and filtered. The filtercake was washed with ether and dried to provide the title compound as a white foam, 39 mg (49%), [α]_{D}²⁵=−28°(c=1, MeOH); HRMS: calcd for C₂₂H₂₉N₃O₂+H⁺, 368.23325; found (ESI, [M+H]+ Obs'd), 368.2334.

### Example 88

### Preparation of 2-[(3R)-1-cyclobutylpyrrolidin-3-yl]-N-cyclopentyl-1-oxo-1,2,3,4-tetrahydroisoquinoline-6-carboxamide hydrochloride

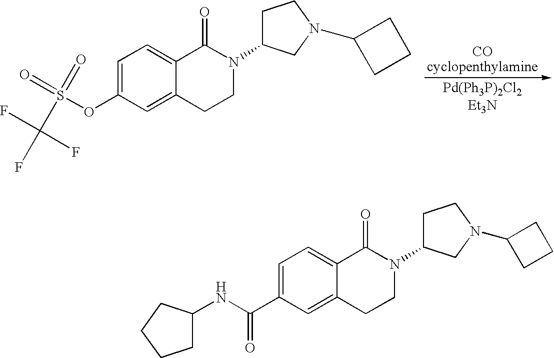

Using essentially the same procedure described in Example 87 and cyclopentyl amine (0.2 g, 2.4 mmol), the title compound 30 mg (33%) was formed as a white solid, mp 124-125° C.; [α]_{D}²⁵=−25° (c=1, MeOH); HRMS: calcd for C₂₃H₃₁N₃O₂+H⁺, 382.24890; found (ESI, [M+H]+ Obs'd), 382.2492.

### Example 89

### Preparation of (R)-2-(1-cyclobutylpyrrolidin-3-yl)-3,3′,4,4′-tetrahydro-6,6′-biisoquinoline-1,1′(2H,2′H)-dione hydrochloride

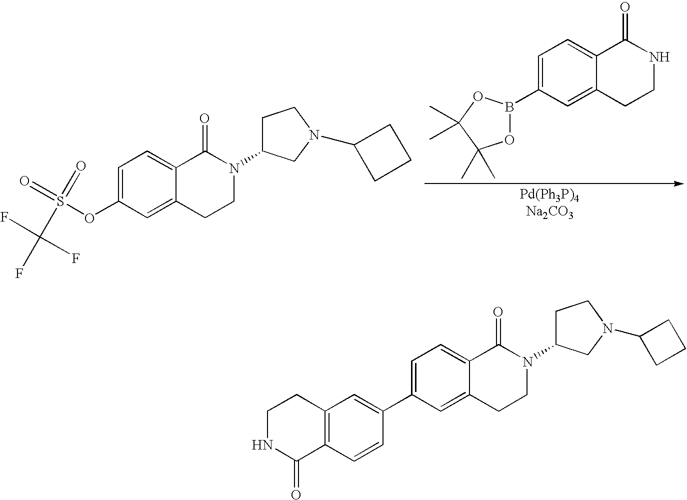

Using essentially the same procedure described in Example 41 and employing 6-(4,4,5,5-tetramethyl-1,3,2-dioxaborolan-2-yl)-3,4-dihydroisoquinolin-1(2H)-one (0.10 g, 0.36 mmol) and (R)-2-(1-cyclobutylpyrrolidin-3-yl)-1-oxo-1,2,3,4-tetrahydroisoqui-nolin-6-yl trifluoromethanesulfonate (0.075 g, 0.18 mmol), the title compound 14 mg (19%) was obtained as a white solid, mp>274° C., [α]_{D}²⁵=−35° (1% SOLUTION, MeOH); HRMS (ES) m/z 416.2337 [M+H]⁺.

### Example 90

### Preparation of (R)-4-(2-(1-cyclobutylpyrrolidin-3-yl)-1-oxo-1,2,3,4-tetrahydro-isoquinolin-6-yl)-N-methylbenzamide hydrochloride

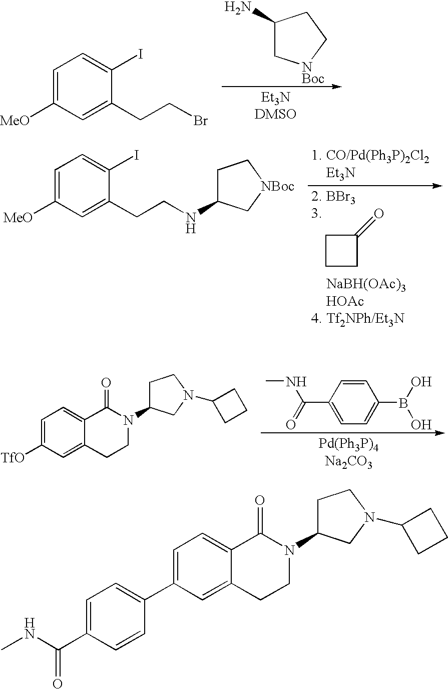

### Step 1: (S)-tert-Butyl 3-(2-iodo-5-methoxyphenethylamino)pyrrolidine-1-carboxylate

Using essentially the same procedure described in step 1 of Example 31 and employing (S)-tert-butyl 3-aminopyrrolidine-1-carboxylate (6.2 mL, 0.038 mol), the title compound 2.94 g (45%) was obtained as a light yellow oil, [α]_{D}²⁵=−6.0° (1% SOLUTION, MeOH); MS (ES) m/z 447.1 [M+H]⁺.

### Step 2: (S)-tert-Butyl 3-(6-methoxy-1-oxo-3,4-dihydroisoquinolin-2(1H)-yl)pyrrolidine-1-carboxylate

Using essentially the same procedure described in step 2 of Example 31 and employing (S)-tert-butyl 3-(2-iodo-5-methoxyphenethylamino)pyrrolidine-1-carboxylate (2.89 g, 6.5 mmol), the title compound 1.5 g (67%) was obtained as a light yellow oil, MS (ES) m/z 369.2 [M+H]⁺.

### Step 3: (S)-6-Hydroxy-2-(pyrrolidin-3-yl)-3,4-dihydroisoquinolin-1(2H)-one

Using essentially the same procedure described in step 3 of Example 31 and employing (S)-tert-butyl 3-(6-methoxy-1-oxo-3,4-dihydroisoquinolin-2(1-yl)pyrrolidine-1-carboxylate (1.49 g, 4.3 mol), the title compound 0.82 g (82%) was obtained as a white foam, [α]_{D}²⁵=10.0° (1% SOLUTION, MeOH); MS (ES) m/z 233.1 [M+H]⁺.

### Step 4: (S)-2-(1-Cyclobutylpyrrolidin-3-yl)-6-hydroxy-3,4-dihydroisoquinolin-1(2H)-one

Using essentially the same procedure described in step 1 of Example 32 and employing (S)-6-hydroxy-2-(pyrrolidin-3-yl)-3,4-dihydroisoquinolin-1(2H)-one (0.77 g, 3.3 mmol), the title compound 0.68 g (72%) was obtained as a white foam, [α]_{D}²⁵=9.0° (1% SOLUTION, MeOH); HRMS (ES) m/z 287.1752 [M+H]⁺.

### Step 5: (S)-2-(1-Cyclobutylpyrrolidin-3-yl)-1-oxo-1,2,3,4-tetrahydroisoquinolin-6-yl trifluoromethanesulfonate

Using essentially the same procedure described in step 1 of Example 44 and employing (S)-2-(1-cyclobutylpyrrolidin-3-yl)-6-hydroxy-3,4-dihydroisoquinolin-1(2H)-one (0.68 g, 2.4 mmol), the title compound 0.68 g (68%) was obtained as a light yellow foam, [α]_{D}²⁵=12.0° (1% SOLUTION, MeOH); HRMS (ES) m/z 419.1247 [M+H]⁺.

### Step 6: (S)-4-(2-(1-Cyclobutylpyrrolidin-3-yl)-1-oxo-1,2,3,4-tetrahydroiso-quinolin-6-yl)-N-methylbenzamide

Using essentially the same procedure described in step 2 of Example 44 and employing (S)-2-(1-cyclobutylpyrrolidin-3-yl)-1-oxo-1,2,3,4-tetrahydroisoquinolin-6-yl trifluoromethanesulfonate (69 mg, 0.17 mmol) and 4-(methylcarbamoyl)phenylboronic acid (0.12 g, 0.34 mmol), the title compound g (%) was obtained as a white solid, mp 265-266° C., [α]_{D}²⁵=30° (1% SOLUTION, MeOH); HRMS (ES) m/z 404.2334 [M+H]⁺.

### Example 91

### Preparation of (R)-4-(2-(1-cyclobutylpyrrolidin-3-yl)-1-oxoisoindolin-5-yl)-N-methylbenzamide hydrochloride

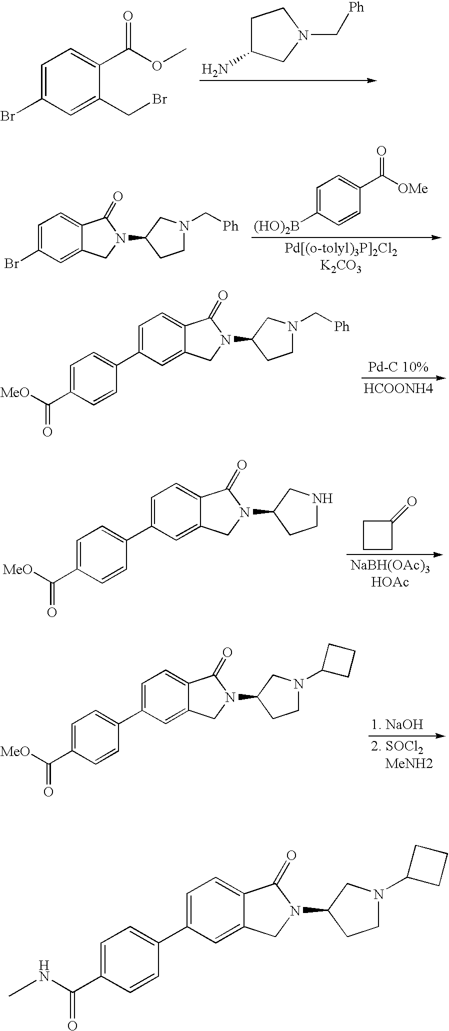

### Step 1: (R)-2-(1-Benzylpyrrolidin-3-yl)-5-bromoisoindolin-1-one

Using essentially the same procedure described in step 3 of Example 22 and employing (R)-1-benzylpyrrolidin-3-amine (1.1 g, 6.5 mmol), the title compound 0.42 g (44%) was obtained as a yellow oil, [α]_{D}²⁵=−38° (1% SOLUTION, MeOH); HRMS (ES) m/z 371.0761 [M+H]⁺.

### Step 2: (R)-Methyl 4-(2-(1-benzylpyrrolidin-3-yl)-1-oxoisoindolin-5-yl)benzoate

Using essentially the same procedure described in step 4 of Example 22 and employing (R)-2-(1-benzylpyrrolidin-3-yl)-5-bromoisoindolin-1-one (0.41 g, 1.1 mmol) and 4-(methoxycarbonyl)phenylboronic acid (0.79 g, 4.4 mmol), the title compound 0.33 g (68%) was obtained as a yellow oil, HRMS (ES) m/z 427.2020 [M+H]⁺.

### Step 3: (R)-Methyl 4-(1-oxo-2-(pyrrolidin-3-yl)isoindolin-5-yl)benzoate

Using essentially the same procedure described in step 6 of Example 54 and employing (R)-methyl 4-(2-(1-benzylpyrrolidin-3-yl)-1-oxoisoindolin-5-yl)benzoate (0.32 g, 0.75 mmol), the title compound 80 mg (32%) was obtained as a white foam, [α]_{D}²⁵=−15° (1% SOLUTION, MeOH); HRMS (ES) m/z 337.1553 [M+H]⁺.

### Step 4: (R)-Methyl 4-(2-(1-cyclobutylpyrrolidin-3-yl)-1-oxoisoindolin-5-yl)benzoate

Using essentially the same procedure described in step 1 of Example 32 and employing (R)-methyl 4-(1-oxo-2-(pyrrolidin-3-yl)isoindolin-5-yl)benzoate (80 mg, 0.24 mmol), the title compound 80 mg (86%) was obtained as a yellow oil, [α]_{D}²⁵=−57° (1% SOLUTION, MeOH); HRMS (ES) m/z 391.2021 [M+H]⁺.

### Step 5: (R)-4-(2-(1-Cyclobutylpyrrolidin-3-yl)-1-oxoisoindolin-5-yl)benzoic acid

Using essentially the same procedure described in step 3 of Example 32 and employing (R)-methyl 4-(2-(1-cyclobutylpyrrolidin-3-yl)-1-oxoisoindolin-5-yl)benzoate (80 mg, 0.2 mmol), the title compound 73 mg (95%) was obtained as a yellow oil, [α]_{D}²⁵=−8° (1% SOLUTION, MeOH); HRMS (ES) m/z 377.1860 [M+H]⁺.

### Step 6: (R)-4-(2-(1-Cyclobutylpyrrolidin-3-yl)-1-oxoisoindolin-5-yl)-N-methylbenzamide hydrochloride

Using essentially the same procedure described in Example 3 and (R)-4-(2-(1-cyclobutylpyrrolidin-3-yl)-1-oxoisoindolin-5-yl)benzoic acid (30 mg, 0.08 mmol) and methyl amine (2.0 M in THF, mL, mmol), the title compound 15 mg (50%) was obtained as a white solid, mp 207-208° C., [α]_{D}²⁵=−41° (1% SOLUTION, MeOH); HRMS (ES) m/z 390.2178 [M+H]⁺.

### Example 92

### Preparation of (R)-2-(1-cyclobutylpyrrolidin-3-yl)-5-(4-(pyrrolidine-1-carbonyl)-phenyl)isoindolin-1-one hydrochloride

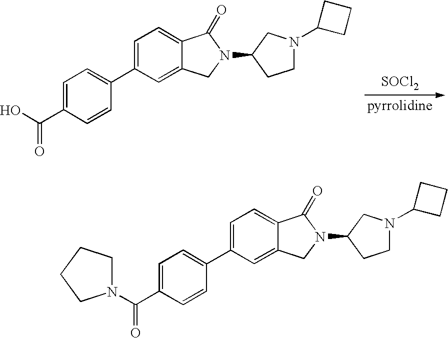

Using essentially the same procedure described in step 6 of Example 91 and pyrrolidine (30 mg, 0.08 mmol), the title compound 16 mg (48%) was obtained as a white solid, mp 220-221° C., [α]_{D}²⁵=−30° (1% SOLUTION, MeOH); HRMS (ES) m/z 430.2496 [M+H]⁺.

### Example 93-102

### Preparation of (R)-4-(2-(1-cyclobutylpyrrolidin-3-yl)-1-oxo-1,2,3,4-tetrahydroiso-quinolin-6-yl)-N-substituted benzamide hydrochloride compounds

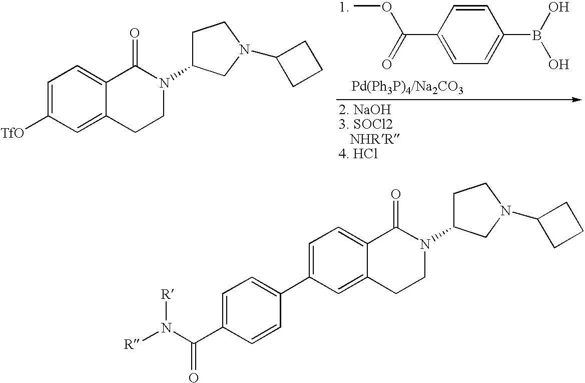

### Step 1: (R)-methyl 4-(2-(1-cyclobutylpyrrolidin-3-yl)-1-oxo-1,2,3,4-tetrahydro-isoquinolin-6-yl)benzoate

Using essentially the same procedure described in Example 41 employing the desired (R)-2-(1-cyclobutylpyrrolidin-3-yl)-1-oxo-1,2,3,4-tetrahydroisoquinolin-6-yl trifluoromethanesulfonate and 4-(methoxycarbonyl)phenylboronic acid, the title compound can be obtained and identified by NMR and mass spectral analyses.

### Step 2: (R)-4-(2-(1-cyclobutylpyrrolidin-3-yl)-1-oxo-1,2,3,4-tetrahydroisoquinolin-6-yl)benzoic acid

Using essentially the same procedure described in Example 14 employing the desired (R)-methyl 4-(2-(1-cyclobutylpyrrolidin-3-yl)-1-oxo-1,2,3,4-tetrahydro-isoquinolin-6-yl)benzoate, the title compound can be obtained and identified by NMR and mass spectral analyses.

### Step 3: (R)-4-(2-(1-cyclobutylpyrrolidin-3-yl)-1-oxo-1,2,3,4-tetrahydro-isoquinolin-6-yl)-N-substituted benzamide

Using essentially the same procedure described in Example 15 employing (R)-4-(2-(1-cyclobutylpyrrolidin-3-yl)-1-oxo-1,2,3,4-tetrahydroisoquinolin-6-yl)benzoic acid and desired amine, the compounds shown in Table XVI can be obtained and identified by NMR and mass spectral analyses.

TABLE XVI

| Ex. | | Ex. | |
|---|---|---|---|
| No. | HNR′R″ | No. | HNR′R″ |
| 93 | i-propylamine | 94 | cyclopropyl amine |
| 95 | azetidine | 96 | cyclobutylamine |
| 97 | 2-fluoroethanaminel | 98 | 2-methoxyethanamine |
| 99 | (S)-1-methoxypropan-2-amine | 100 | 2-isopropoxyethanamine |
| 101 | morpholine | 102 | piperidine |

### Example 103-112

### Preparation of (R)-4-(2-(1-cyclobutylpyrrolidin-3-yl)-1-oxo-1,2,3,4-tetrahydro-isoquinolin-6-yl)-3-fluoro-N-substituted benzamide hydrochloride compounds

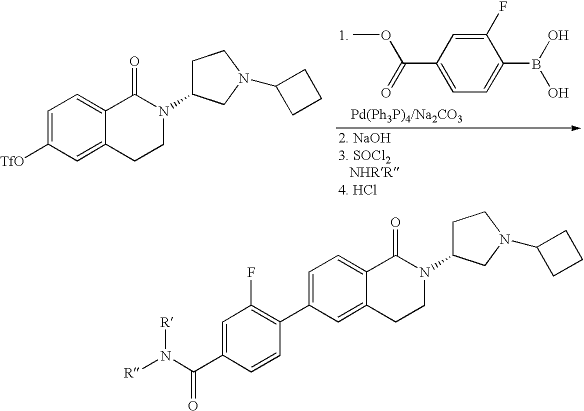

Using essentially the same procedure described in step 5 of Example 73 employing desired amines, the compounds shown in Table XVII can be obtained and identified by NMR and mass spectral analyses.

TABLE XVII

| Ex. | | Ex. | |
|---|---|---|---|
| No. | HNR′R″ | No. | HNR′R″ |
| 103 | i-propylamine | 104 | cyclopropyl amine |
| 105 | azetidine | 106 | cyclobutylamine |
| 107 | 2-fluoroethanamine | 108 | 2-methoxyethanamine |
| 109 | (S)-1-methoxypropan-2-amine | 110 | 2-isopropoxyethanamine |
| 111 | morpholine | 112 | piperidine |

### Evaluation of Methyl Histamine Binding in Human Histamine H₃ Receptor Cell Line

The affinity of test compounds for the histamine 3 (H₃) receptor is evaluated in the following manner. Stably transfected HEK293T cells are grown in DMEM containing 10% heat inactivated FBS and G-418 (500 ug/ml). Cells are scraped from the plate, transferred to centrifuge tubes, washed one time in PBS by centrifugation in a Sorvall RT7 Plus centrifuge (2000 rpm 10 minutes, 4° C.). The resulting pellets are stored at −80° C. until ready for use. Cells are re-suspended in buffer (50 mM Tris pH=7.5) and placed in a Dounce homogenizer, douncing ten times to homogenize cells. The homogenate is spun down by centrifugation (Sorvall RT7 Plus, 1800 rpm 10 minutes, 4° C.). The supernatant is placed in a Corex tube and spun down by centrifugation (Sorvall RC 5c Plus, 17,000 rpm 20 minutes, 4° C.). The pellet is resuspended in buffer (50 mM Tris, pH 7.5). Protein concentration (ug/ul) is determined using the Micro-BCA Protein Determination. The binding assay is set up in a 96 well microtiter plate in a total volume of 250 uL. Non-specific binding is determined in the presence of 10 uM clobenpropit. The final radioligand concentration is 1 nM. The test compound is serially diluted using the Beckman Biomek2000 to a final approximate range of 100 uM to 100 pM. Membranes are suspended in buffer, homogenized in 2 bursts of ten seconds using a Vitris mechanical homogenizer set at power setting 5. Ten μg of membranes are added to each well. Following a one hour incubation at 30° C., the reaction is terminated by the addition of ice cold buffer and rapid filtration with a Packard Filtermate Harvester through a GF/B filter pre-soaked with 1% PEI for one hour. The plate is dried for one hour at 37° C. and 60 μL Microscint Scintillant is added to each well. The CPM per well is measured on a Packard Top Count NXT. Ki values are determined in nM. The Ki is calculated from the IC₅₀ (i.e. the concentration of competing ligand which displaces 50% of the specific binding of the radioligand). CPM values are expressed as % specific binding and plotted vs compound concentration. A curve is fitted using a four-parameter logistic fit and the IC₅₀ value is determined. The Ki is calculated from this using the Cheng-Prusoff equation: pKi=IC₅₀/1+(L/Kd) where L=concentration of free radioligand used in the assay, and Kd is the dissociation constant of the radioligand for the receptor. L is determined for each experiment by counting an aliquot of the diluted radioligand (corresponding to that added to each well) and the Kd has previously been determined under identical conditions for this cell line/radioligand.

### Cyclic AMP Assay for Histamine Receptor H₃ Antagonism Activity.

Stable H₃ cells are maintained in tissue culture flask in DMEM with high glucose, 10% FBS, 1× pen/strep, 500 ug/ml GY18, until experiment. Culture media is removed and cells are washed twice with PBS w/Ca++ and Mg++ plus 500 μM IBMX. Cells are then detached by tapping on the side of the flask and resuspend in the same buffer. Two thousand cells/well are incubated with 1 μM histamine plus 10 μM forskolin plus various concentrations of compounds in a total volume of 30 μL in 96 well plates for 30 min at 30° C. Final test compound concentrations range from 10-4M to 10-9.5M at full log dilutions. Cyclic AMP levels are measured using HitHunter cAMP kit from Discoverx, cat#900041 according to manufacturer's instruction. Chemiluminescence signals are detected using Top Count (Packard).

Cyclic AMP levels in control cells receiving 10 μM forskolin plus 100 nM histamine are considered 0%, and in cells receiving 10 μM forskolin plus 100 nM histamine plus 1 μM clobenpropit are considered 100%. Data are expressed as % control and analyzed using Prizm soft ware. The Kb values are calculated using the following equation, KB=EC₅₀ or IC₅₀/[1+(ligand/Kd)]. The data are shown in Table IV, below.

TABLE IV

| Ex No | H₃ Binding Ki (nM) |
|---|---|
| 1 | D |
| 2 | D |
| 3 | B |
| 4 | A |
| 5 | A |
| 6 | B |
| 7 | A |
| 8 | A |
| 9 | B |
| 10 | B |
| 11 | C |
| 12 | A |
| 13 | B |
| 14 | B |
| 15 | A |
| 16 | A |
| 17 | A |
| 18 | A |
| 19 | A |
| 20 | A |
| 21 | A |
| 22 | E |
| 23 | E |
| 24 | E |
| 25 | E |
| 26 | D |
| 27 | C |
| 28 | C |
| 29 | B |
| 30 | C |
| 32 | E |
| 33 | A |
| 34 | A |
| 35 | B |
| 36 | D |
| 37 | A |
| 38 | B |
| 39 | A |
| 41 | A |
| 42 | A |
| 43 | A |
| 44 | A |
| 45 | A |
| 46 | A |
| 47 | A |
| 48 | A |
| 49 | A |
| 50 | — |
| 51 | — |
| 52 | A |
| 53 | A |
| 55 | D |
| 56 | A |
| 57 | A |
| 58 | A |
| 59 | B |
| 60 | A |
| 61 | E |
| 62 | C |
| 65 | A |
| 66 | A |
| 67 | A |
| 70 | B |
| 71 | B |
| 72 | E |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | A |
| 87 | C |
| 88 | B |
| 89 | A |
| 90 | A |
| 91 | B |
| 92 | A |

For Table IV
A = ≦10 nM
B = 10.1 nM-25.0 nM
C = 25.1 nM-50.0 nM
D = 50.1 nM-100 nM
E = >100 nM

What is claimed is:

1. A compound of formula I 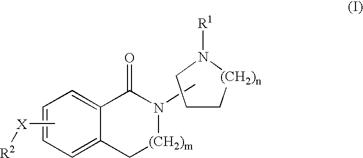
wherein
X is (CR³R⁴)ₚ, CO or O;
m is 0, 1 or 2;
n is 0, 1, 2 or 3;
p is 0, 1 or 2;
R¹ is an alkyl or cycloalkyl group each group optionally substituted;
R² is NR⁵R⁶ or an alkyl, cycloalkyl, cycloheteroalkyl, aryl or heteroaryl group each group optionally substituted with the proviso that when X is O then R² must be other than NR⁵R⁶;
R³ and R⁴ are each independently H, halogen or an optionally substituted alkyl or cycloalkyl group; and
R⁵ and R⁶ each independently H or an alkyl, alkenyl, alkoxy, cycloalkyl, cycloheteroalkyl, aryl or heteroaryl group each group optionally substituted or R⁵ and R⁶ may be taken together with the atom to which they are attached to form an optionally substituted 4- to 7-membered ring optionally containing one or two additional heteroatoms selected from N, O or S or an optionally substituted fused bicyclic or tricyclic 9- to 15-membered aromatic ring system optionally containing one to three additional heteroatoms selected from N, O or S; or
a stereoisomer thereof or a pharmaceutically acceptable salt thereof;
provided that R¹ is not diphenylpropyl.

2. The compound of claim 1 wherein n is 1 or 2.

3. The compound of claim 1 wherein R¹ is optionally substituted cycloalkyl.

4. The compound of claim 1 wherein R¹ is C₁-C₄ alkyl.

5. The compound of claim 1 wherein R¹ is a C₃-C₆ cycloalkyl.

6. The compound of claim 1 having the structure of formula Ix: 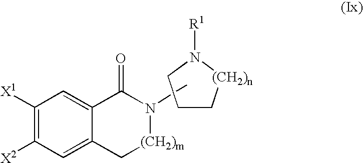
wherein,
X¹ is H and X² is —X—R²; or
X¹ is —X—R²and X² is H.

7. The compound of claim 1 having the structure of formula Ia 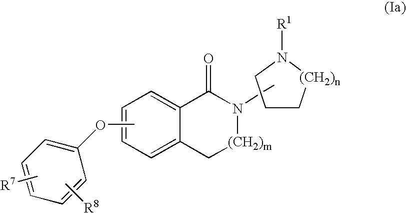
wherein
m is 0, 1 or 2;
n is 0, 1, 2 or 3;
R¹ is an alkyl or cycloalkyl group each group optionally substituted;
R⁷ and R⁸ are each independently H, halogen, CN, CONR⁹R¹⁰, OR¹¹, CO₂R¹¹, COR¹¹, or an alkyl, haloalkyl or cycloalkyl group each group optionally substituted; R⁹ and R¹⁰ are each independently H or an alkyl, haloalkyl, cycloalkyl, aryl or heteroaryl group each group optionally substituted or R⁹ and R¹⁰ may be taken together with the atom to which they are attached to form an optionally substituted 5- to 7-membered ring optionally containing one or two additional heteroatoms selected from N, O or S; and
R¹¹ is H or an alkyl, haloalkyl, cycloalkyl, alkenyl, alkynyl, aryl or heteroaryl group each group optionally substituted; or
a stereoisomer thereof or a pharmaceutically acceptable salt thereof.

8. The compound of claim 1 wherein the azacyclic ring is attached at the 3-position of said azacyclic ring.

9. The compound of claim 1 wherein m is 1.

10. The compound of claim 7 wherein the azacyclic ring is attached at the 3-position of said azacyclic ring; R⁷ is CONR⁹R¹⁰; and R⁸ is H.

11. The compound of claim 1 wherein R² is an optionally substituted aminocarbonylphenyl or cycloheteroalkylcarbonylphenyl group.

12. The compound of claim 1 wherein X is (CR³R⁴)ₚ and p is 0.

13. The compound of claim 1 wherein R² is selected from the group consisting of methyloxycarbonylphenyl, carboxyphenyl, aminocarbonylphenyl, alkylaminocarbonylphenyl, cycloalkylaminocarbonylphenyl, N,N-dialkylaminocarbonylphenyl, carboxyphenylalkyl, aminocarbonylphenylalkyl, alkylaminocarbonylphenylalkyl, N,N-dialkylaminocarbonylphenylalkyl, cycloalkylaminocarbonylphenylalkyl, cyanophenyl, cycloheteroalkylcarbonylphenyl, aminocarbonylhalophenyl, alkylaminocarbonylhalophenyl, N,N-dialkylaminocarbonylhalophenyl, cycloheteroalkylcarbonylhalophenyl, halophenyl, phenyl, dihalophenyl, alkylaminocarbonylhalophenyl, pyrrolidine-1-carbonylphenyl, and aminoalkoxyalkyl.

14. The compound of claim 1 selected from the group consisting essentially of:
4-{[2-(1-Cyclobutylpiperidin-4-yl)-1-oxo-1,2,3,4-tetrahydroisoquinolin-6-yl]oxy}benzoic acid;
Methyl 4-{[2-(1-cyclobutylpiperidin-4-yl)-1-oxo-1,2,3,4-tetrahydroisoquinolin-6-yl]oxy}benzoate;
4-{[2-(1-Cyclobutylpiperidin-4-yl)-1-oxo-1,2,3,4-tetrahydroisoquinolin-6-yl]oxy}benzamide;
4-{[2-(1-Cyclobutylpiperidin-4-yl)-1-oxo-1,2,3,4-tetrahydroisoquinolin-6-yl]oxy}-N-methylbenzamide;
4-{[2-(1-Cyclobutylpiperidin-4-yl)-1-oxo-1,2,3,4-tetrahydroisoquinolin-6-yl]oxy}-N-ethylbenzamide;
4-{[2-(1-Cyclobutylpiperidin-4-yl)-1-oxo-1,2,3,4-tetrahydroisoquinolin-6-yl]oxy}-N-isopropylbenzamide;
4-{[2-(1-Cyclobutylpiperidin-4-yl)-1-oxo-1,2,3,4-tetrahydroisoquinolin-6-yl]oxy}-N-cyclopropylbenzamide;
N-Cyclobutyl-4-{[2-(1-cyclobutylpiperidin-4-yl)-1-oxo-1,2,3,4-tetrahydroisoquinolin-6-yl]oxy}benzamide;
4-{[2-(1-Cyclobutylpiperidin-4-yl)-1-oxo-1,2,3,4-tetrahydroisoquinolin-6-yl]oxy}-N-cyclopentylbenzamide;
4-{[2-(1-Cyclobutylpiperidin-4-yl)-1-oxo-1,2,3,4-tetrahydroisoquinolin-6-yl]oxy}-N,N-dimethylbenzamide;
4-{[2-(1-Cyclobutylpiperidin-4-yl)-1-oxo-1,2,3,4-tetrahydroisoquinolin-6-yl]oxy}-N,N-diethylbenzamide;
2-(1-Cyclobutylpiperidin-4-yl)-6-[4-(pyrrolidin-1-ylcarbonyl)phenoxy]-3,4-dihydroisoquinolin-1(2H)-one;
Methyl 4-({[2-(1-Cyclobutylpiperidin-4-yl)-1-oxo-1,2,3,4-tetrahydroisoquinolin-6-yl]oxy}methyl)benzoate;
4-({[2-(1-Cyclobutylpiperidin-4-yl)-1-oxo-1,2,3,4-tetrahydroisoquinolin-6-yl]oxy}methyl)benzoic acid;
4-({[2-(1-Cyclobutylpiperidin-4-yl)-1-oxo-1,2,3,4-tetrahydroisoquinolin-6-yl]oxy}methyl)benzamide;
4-({[2-(1-Cyclobutylpiperidin-4-yl)-1-oxo-1,2,3,4-tetrahydroisoquinolin-6-yl]oxy}methyl)-N-methylbenzamide;
4-({[2-(1-Cyclobutylpiperidin-4-yl)-1-oxo-1,2,3,4-tetrahydroisoquinolin-6-yl]oxy}methyl)-N-ethylbenzamide
4-({[2-(1-Cyclobutylpiperidin-4-yl)-1-oxo-1,2,3,4-tetrahydroisoquinolin-6-yl]oxy}methyl)-N,N-dimethylbenzamide;
N-Cyclobutyl-4-({[2-(1-cyclobutylpiperidin-4-yl)-1-oxo-1,2,3,4-tetrahydroisoquinolin-6-yl]oxy}methyl)benzamide;
2-(1-Cyclobutylpiperidin-4-yl)-6-{[4-(pyrrolidin-1-ylcarbonyl)benzyl]oxy}-3,4-dihydroisoquinolin-1(2H)-one;
4-({[2-(1-Cyclobutylpiperidin-4-yl)-1-oxo-1,2,3,4-tetrahydroisoquinolin-6-yl]oxy}methyl)-N-cyclopentylbenzamide;
4-(1-Oxo-2-piperidin-4-yl-2,3-dihydro-1H-isoindol-5-yl)benzonitrile;
4-[2-(1-Methylpiperidin-4-yl)-1-oxo-2,3-dihydro-1H-isoindol-5-yl]benzonitrile;
4-[2-(1-Ethylpiperidin-4-yl)-1-oxo-2,3-dihydro-1H-isoindol-5-yl]benzonitrile;
4-[1-Oxo-2-(1-propylpiperidin-4-yl)-2,3-dihydro-1H-isoindol-5-yl]benzonitrile;
4-{2-[1-(Cyclopropylmethyl)piperidin-4-yl]-1-oxo-2,3-dihydro-1H-isoindol-5-yl}benzonitrile;
4-{2-[1-(Cyclopentylmethyl)piperidin-4-yl]-1-oxo-2,3-dihydro-1H-isoindol-5-yl}benzonitrile;
4-[2-(1-Cyclobutylpiperidin-4-yl)-1-oxo-2,3-dihydro-1H-isoindol-5-yl]benzonitrile;
4-[2-(1-Cyclopentylpiperidin-4-yl)-1-oxo-2,3-dihydro-1H-isoindol-5-yl]benzonitrile;
4-[2-(1-Cyclohexylpiperidin-4-yl)-1-oxo-2,3-dihydro-1H-isoindol-5-yl]benzonitrile;
4-({2-[(3R)-1-cyclobutylpyrrolidin-3-yl]-1-oxo-1,2,3,4-tetrahydroisoquinolin-6-yl}oxy)benzoic acid;
4-({2-[(3R)-1-cyclobutylpyrrolidin-3-yl]-1-oxo-1,2,3,4-tetrahydroisoquinolin-6-yl}oxy)-N-methylbenzamide;
4-({2-[(3R)-1-cyclobutylpyrrolidin-3-yl]-1-oxo-1,2,3,4-tetrahydroisoquinolin-6-yl}oxy)-N-ethylbenzamide;
2-[(3R)-1-cyclobutylpyrrolidin-3-yl]-6-[4-(pyrrolidin-1-ylcarbonyl)phenoxy]-3,4-dihydroisoquinolin-1(2H)-one;
4-({2-[(3R)-1-cyclopentylpyrrolidin-3-yl]-1-oxo-1,2,3,4-tetrahydroisoquinolin-6-yl}oxy)benzoic acid;
4-({2-[(3R)-1-cyclopentylpyrrolidin-3-yl]-1-oxo-1,2,3,4-tetrahydroisoquinolin-6-yl}oxy)-N-methylbenzamide;
4-({2-[(3R)-1-cyclopentylpyrrolidin-3-yl]-1-oxo-1,2,3,4-tetrahydroisoquinolin-6-yl}oxy)-N-ethylbenzamide;
2-[(3R)-1-cyclopentylpyrrolidin-3-yl]-6-[4-(pyrrolidin-1-ylcarbonyl)phenoxy]-3,4-dihydroisoquinolin-1(2H)-one;
4-{2-[(3R)-1-cyclopentylpyrrolidin-3-yl]-1-oxo-1,2,3,4-tetrahydroisoquinolin-6-yl}-N-methylbenzamide;
4-{2-[(3R)-1-cyclopentylpyrrolidin-3-yl]-1-oxo-1,2,3,4-tetrahydroisoquinolin-6-yl}-N-ethylbenzamide;
2-[(3R)-1-cyclopentylpyrrolidin-3-yl]-6-[4-(pyrrolidin-1-ylcarbonyl)phenyl]-3,4-dihydroisoquinolin-1(2H)-one;
4-{2-[(3R)-1-cyclobutylpyrrolidin-3-yl]-1-oxo-1,2,3,4-tetrahydroisoquinolin-6-yl}-N-methylbenzamide;
4-{2-[(3R)-1-cyclobutylpyrrolidin-3-yl]-1-oxo-1,2,3,4-tetrahydroisoquinolin-6-yl}-N-ethylbenzamide;
2-[(3R)-1-cyclobutylpyrrolidin-3-yl]-6-[4-(pyrrolidin-1-ylcarbonyl)phenyl]-3,4-dihydroisoquinolin-1(2H)-one;
2-[(3R)-1-(cyclopentylmethyl)pyrrolidin-3-yl]-6-[4-(pyrrolidin-1-ylcarbonyl)phenyl]-3,4-dihydroisoquinolin-1(2H)-one;
2-[(3R)-1-methylpyrrolidin-3-yl]-6-[4-(pyrrolidin-1-ylcarbonyl)phenyl]-3,4-dihydroisoquinolin-1(2H)-one;
2-[(3R)-1-(cyclopropylmethyl)pyrrolidin-3-yl]-6-[4-(pyrrolidin-1-ylcarbonyl)phenyl]-3,4-dihydroisoquinolin-1(2H)-one;
2-[(3R)-1-(furan-3-ylmethyl)pyrrolidin-3-yl]-6-[4-(pyrrolidin-1-ylcarbonyl)phenyl]-3,4-dihydroisoquinolin-1(2H)-one;
2-[(3R)-1-cyclohexylpyrrolidin-3-yl]-6-[4-(pyrrolidin-1-ylcarbonyl)phenyl]-3,4-dihydroisoquinolin-1(2H)-one;
2-[(3R)-1-(furan-2-ylmethyl)pyrrolidin-3-yl]-6-[4-(pyrrolidin-1-ylcarbonyl)phenyl]-3,4-dihydroisoquinolin-1(2H)-one;
6-[4-(pyrrolidin-1-ylcarbonyl)phenyl]-2-[(3R)-1-(thiophen-2-ylmethyl)pyrrolidin-3-yl]-3,4-dihydroisoquinolin-1(2H)-one;
2-[(3R)-1-(1-methylethyl)pyrrolidin-3-yl]-6-[4-(pyrrolidin-1-ylcarbonyl)phenyl]-3,4-dihydroisoquinolin-1(2H)-one;
2-(1-cyclobutylpiperidin-4-yl)-6-[4-(pyrrolidin-1-ylcarbonyl)phenyl]-3,4-dihydroisoquinolin-1(2H)-one;
2-[(3R)-piperidin-3-yl]-6-[4-(pyrrolidin-1-ylcarbonyl)phenyl]-3,4-dihydroisoquinolin-1(2H)-one;
2-(1-cyclopentylpiperidin-4-yl)-6-[4-(pyrrolidin-1-ylcarbonyl)phenyl]-3,4-dihydroisoquinolin-1(2H)-one;
2-[(3R)-1-cyclopentylpiperidin-3-yl]-6-[4-(pyrrolidin-1-ylcarbonyl)phenyl]-3,4-dihydroisoquinolin-1(2H)-one;
2-[(3R)-1-cyclobutylpiperidin-3-yl]-6-[4-(pyrrolidin-1-ylcarbonyl)phenyl]-3,4-dihydroisoquinolin-1(2H)-one;
2-[(3R)-1-cyclobutylpyrrolidin-3-yl]-6-(2-fluoro-4-{[(2S)-2-methylpyrrolidin-1-yl]carbonyl}phenyl)-3,4-dihydroisoquinolin-1(2H)-one;
N-cyclobutyl-4-{2-[(3R)-1-cyclobutylpyrrolidin-3-yl]-1-oxo-1,2,3,4-tetrahydroisoquinolin-6-yl}-3-fluorobenzamide;
4-{2-[(3R)-1-cyclobutylpyrrolidin-3-yl]-1-oxo-1,2,3,4-tetrahydroisoquinolin-6-yl}-3-fluoro-N-methylbenzamide;
2-[(3R)-1-cyclobutylpyrrolidin-3-yl]-6-(2-fluoro-4-{[(2R)-2-methylpyrrolidin-1-yl]carbonyl}phenyl)-3,4-dihydroisoquinolin-1(2H)-one;
4-{2-[(3R)-1-cyclobutylpyrrolidin-3-yl]-1-oxo-1,2,3,4-tetrahydroisoquinolin-6-yl}-N-ethyl-3-fluorobenzamide;
4-{2-[(3R)-1-cyclobutylpyrrolidin-3-yl]-1-oxo-1,2,3,4-tetrahydroisoquinolin-6-yl}-3-fluoro-N,N-dimethylbenzamide;
2-[(3R)-1-cyclobutylpyrrolidin-3-yl]-6-[2-fluoro-4-(pyrrolidin-1-ylcarbonyl)phenyl]-3,4-dihydroisoquinolin-1(2H)-one;
4-{2-[(3R)-1-cyclobutylpyrrolidin-3-yl]-1-oxo-1,2,3,4-tetrahydroisoquinolin-6-yl}-N-cyclopentyl-3-fluorobenzamide;
4-{2-[(3R)-1-cyclobutylpyrrolidin-3-yl]-1-oxo-1,2,3,4-tetrahydroisoquinolin-6-yl}-N-cyclopropyl-3-fluorobenzamide;
2-[(3R)-1-cyclobutylpyrrolidin-3-yl]-6-[4-fluoro-3-(pyrrolidin-1-ylcarbonyl)phenyl]-3,4-dihydroisoquinolin-1(2H)-one;
2-[(3R)-1-cyclobutylpyrrolidin-3-yl]-6-[3-fluoro-4-(pyrrolidin-1-ylcarbonyl)phenyl]-3,4-dihydroisoquinolin-1(2H)-one;
6-[3-chloro-4-(pyrrolidin-1-ylcarbonyl)phenyl]-2-[(3R)-1-cyclobutylpyrrolidin-3-yl]-3,4-dihydroisoquinolin-1(2H)-one;
2-(1-isopropylpiperidin-4-yl)-6-[4-(pyrrolidin-1-ylcarbonyl)phenyl]-3,4-dihydroisoquinolin-1(2H)-one;
2-[(3R)-1-isopropylpiperidin-3-yl]-6-[4-(pyrrolidin-1-ylcarbonyl)phenyl]-3,4-dihydroisoquinolin-1(2H)-one;
2-[(3R)-1-cyclobutylpyrrolidin-3-yl]-6-[3-(pyrrolidin-1-ylcarbonyl)phenyl]-3,4-dihydroisoquinolin-1(2H)-one;
4-{2-[(3R)-1-cyclobutylpyrrolidin-3-yl]-1-oxo-1,2,3,4-tetrahydroisoquinolin-6-yl}-N,N-dimethylbenzamide;
2-[(3R)-1-cyclobutylpyrrolidin-3-yl]-N-cyclopentyl-1-oxo-1,2,3,4-tetrahydroisoquinoline-6-carboxamide;
2-[(3R)-1-cyclobutylpyrrolidin-3-yl]-6-(pyrrolidin-1-ylcarbonyl)-3,4-dihydroisoquinolin-1(2H)-one;
N-ethyl-3-fluoro-4-{2-[(3R)-1-isopropylpyrrolidin-3-yl]-1-oxo-1,2,3,4-tetrahydroisoquinolin-6-yl}benzamide;
3-fluoro-4-{2-[(3R)-1-isopropylpyrrolidin-3-yl]-1-oxo-1,2,3,4-tetrahydroisoquinolin-6-yl}-N-methylbenzamide;
N-ethyl-4-{2-[(3R)-1-isopropylpyrrolidin-3-yl]-1-oxo-1,2,3,4-tetrahydroisoquinolin-6-yl}benzamide;
6-[2-fluoro-4-(pyrrolidin-1-ylcarbonyl)phenyl]-2-[(3R)-1-isopropylpyrrolidin-3-yl]-3,4-dihydroisoquinolin-1(2H)-one;
2-[(3R)-1-cyclobutylpyrrolidin-3-yl]-6-phenyl-3,4-dihydroisoquinolin-1(2H)-one;
2-[(3R)-1-cyclobutylpyrrolidin-3-yl]-6-(4-fluorophenyl)-3,4-dihydroisoquinolin-1(2H)-one;
2-[(3R)-1-cyclobutylpyrrolidin-3-yl]-5-[4-(pyrrolidin-1-ylcarbonyl)phenyl]isoindolin-1-one;
4-{2-[(3R)-1-cyclobutylpyrrolidin-3-yl]-1-oxo-2,3-dihydro-1H-isoindol-5-yl}-N-methylbenzamide;
2-[(3R)-1-cyclobutylpyrrolidin-3-yl]-3,3′,4,4′-tetrahydro-6,6′-biisoquinoline-1,1′(2H,2′H)-dione;
2-[(3R)-1-benzylpyrrolidin-3-yl]-6-[4-(pyrrolidin-1-ylcarbonyl)phenyl]-3,4-dihydroisoquinolin-1(2H)-one;
2-[(³R)-pyrrolidin-3-yl]-6-[4-(pyrrolidin-1-ylcarbonyl)phenyl]-3,4-dihydroisoquinolin-1(2H)-one;
4-{2-[(3S)-1-cyclobutylpyrrolidin-3-yl]-1-oxo-1,2,3,4-tetrahydroisoquinolin-6-yl}-N-methylbenzamide;
2-(1-benzylpiperidin-4-yl)-6-[4-(pyrrolidin-1-ylcarbonyl)phenyl]-3,4-dihydroisoquinolin-1(2H)-one;
2-[(3R)-1-benzylpiperidin-3-yl]-6-[4-(pyrrolidin-1-ylcarbonyl)phenyl]-3,4-dihydroisoquinolin-1(2H)-one;
(R)-4-(2-(1-cyclobutylpyrrolidin-3-yl)-1-oxo-1,2,3,4-tetrahydroisoquinolin-6-yl)-N-isopropylbenzamide;
(R)-4-(2-(1-cyclobutylpyrrolidin-3-yl)-1-oxo-1,2,3,4-tetrahydroisoquinolin-6-yl)-N-cyclopropylbenzamide;
(R)—N-cyclobutyl-4-(2-(1-cyclobutylpyrrolidin-3-yl)-1-oxo-1,2,3,4-tetrahydroisoquinolin-6-yl)benzamide;
(R)-6-(4-(azetidine-1-carbonyl)phenyl)-2-(1-cyclobutylpyrrolidin-3-yl)-3,4-dihydroisoquinolin-1(2H)-one;
(R)-4-(2-(1-cyclobutylpyrrolidin-3-yl)-1-oxo-1,2,3,4-tetrahydroisoquinolin-6-yl)-N-(2-fluoroethyl)benzamide;
(R)-4-(2-(1-cyclobutylpyrrolidin-3-yl)-1-oxo-1,2,3,4-tetrahydroisoquinolin-6-yl)-N-(2-methoxyethyl)benzamide;
4-(2-((R)-1-cyclobutylpyrrolidin-3-yl)-1-oxo-1,2,3,4-tetrahydroisoquinolin-6-yl)-N—((S)-1-methoxypropan-2-yl)benzamide;
(R)-4-(2-(1-cyclobutylpyrrolidin-3-yl)-1-oxo-1,2,3,4-tetrahydroisoquinolin-6-yl)-N-(2-isopropoxyethyl)benzamide;
(R)-2-(1-cyclobutylpyrrolidin-3-yl)-6-(4-(morpholine-4-carbonyl)phenyl)-3,4-dihydroisoquinolin-1(2H)-one;
(R)-2-(1-cyclobutylpyrrolidin-3-yl)-6-(4-(piperidine-1-carbonyl)phenyl)-3,4-dihydroisoquinolin-1(2H)-one;
(R)-2-(1-cyclobutylpyrrolidin-3-yl)-6-(2-fluoro-4-(piperidine-1-carbonyl)phenyl)-3,4-dihydroisoquinolin-1(2H)-one;
(R)-2-(1-cyclobutylpyrrolidin-3-yl)-6-(2-fluoro-4-(morpholine-4-carbonyl)phenyl)-3,4-dihydroisoquinolin-1(2H)-one;
(R)-4-(2-(1-cyclobutylpyrrolidin-3-yl)-1-oxo-1,2,3,4-tetrahydroisoquinolin-6-yl)-3-fluoro-N-(2-isopropoxyethyl)benzamide;
4-(2-((R)-1-cyclobutylpyrrolidin-3-yl)-1-oxo-1,2,3,4-tetrahydroisoquinolin-6-yl)-3-fluoro-N—((S)-1-methoxypropan-2-yl)benzamide;
(R)-4-(2-(1-cyclobutylpyrrolidin-3-yl)-1-oxo-1,2,3,4-tetrahydroisoquinolin-6-yl)-3-fluoro-N-(2-methoxyethyl)benzamide;
(R)-4-(2-(1-cyclobutylpyrrolidin-3-yl)-1-oxo-1,2,3,4-tetrahydroisoquinolin-6-yl)-3-fluoro-N-(2-fluoroethyl)benzamide;
(R)—N-cyclobutyl-4-(2-(1-cyclobutylpyrrolidin-3-yl)-1-oxo-1,2,3,4-tetrahydroisoquinolin-6-yl)-3-fluorobenzamide;
(R)-4-(2-(1-cyclobutylpyrrolidin-3-yl)-1-oxo-1,2,3,4-tetrahydroisoquinolin-6-yl)-N-cyclopropyl-3-fluorobenzamide;
(R)-4-(2-(1-cyclobutylpyrrolidin-3-yl)-1-oxo-1,2,3,4-tetrahydroisoquinolin-6-yl)-3-fluoro-N-isopropylbenzamide;
(R)-6-(4-(azetidine-1-carbonyl)-2-fluorophenyl)-2-(1-cyclobutylpyrrolidin-3-yl)-3,4-dihydroisoquinolin-1(2H)-one;
a stereoisomer thereof; and
a pharmaceutically acceptable salt thereof.

15. A method for the treatment of a patient suffering from a cognitive disorder related to or affected by the Histamine-3 (H₃) receptor comprising administering to the patient a compound of formula I 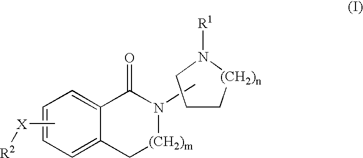
wherein
X is (CR³R⁴)ₚ, CO or O;
m is 0, 1 or 2;
n is 0, 1, 2 or 3;
p is 0, 1 or 2;
R¹ is an alkyl or cycloalkyl group each group optionally substituted;
R² is NR⁵R⁶ or an alkyl, cycloalkyl, cycloheteroalkyl, aryl or heteroaryl group each group optionally substituted with the proviso that when X is O then R² must be other than NR⁵R⁶;
R³ and R⁴ are each independently H, halogen or an optionally substituted alkyl or cycloalkyl group; and
R⁵ and R⁶ each independently H or an alkyl, alkenyl, alkoxy, cycloalkyl, cycloheteroalkyl, aryl or heteroaryl group each group optionally substituted or R⁵ and R⁶ may be taken together with the atom to which they are attached to form an optionally substituted 4- to 7-membered ring optionally containing one or two additional heteroatoms selected from N, O or S or an optionally substituted fused bicyclic or tricyclic 9- to 15-membered aromatic ring system optionally containing one to three additional heteroatoms selected from N, O or S; or
a stereoisomer thereof or a pharmaceutically acceptable salt thereof.

16. The method of claim 15, wherein said disorder is a neurodegenerative disorder.

17. The method of 15, wherein said disorder is mild cognitive impairment (MCI), dementia, delirium, amnestic disorder, Alzheimer's disease (AD), Parkinson's disease (PD), Huntington's disease (HD), memory disorder, memory deficits associated with depression, schizophrenia, a psychotic disorder, paranoia, mano-depressive illness, attention deficit hyperactivity disorder (ADHD), dyslexia, developmental disorders, Down's syndrome, Fragile X syndrome, loss of executive function, loss of learned information, vascular dementia, cognitive decline, neurodegenerative disorder, HIV-induced dimentia, head trauma, Pick's disease, Creutzfeldt-Jakob disease, Body dementia, vascular dementia, surgical procedure-induced cognitive dysfunction, traumatic brain injury or stroke.

18. The method of claim 15, wherein said disorder is selected from the group consisting of: Alzheimer's disease, attention deficit disorder, schizophrenia, cognitive dysfunction in schizophrenia, Parkinsons' disease, frontal temporal dementia or depression.

19. A pharmaceutical composition which comprises a pharmaceutically acceptable carrier and an effective amount of a compound of claim 1.

20. A process for the preparation of a compound of formula Ia′ 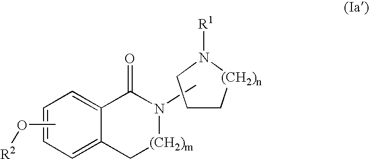
wherein
m is 0, 1 or 2;
n is 0, 1, 2 or 3;
R¹ is an alkyl or cycloalkyl group each group optionally substituted;
R² is an alkyl, cycloalkyl, cycloheteroalkyl, aryl or heteroaryl group each group optionally substituted;
which process comprises reacting a compound of formula II 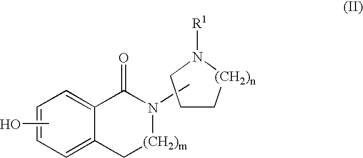
wherein R¹, m and n are as described hereinabove for formula Ia′ with a compound, R²-Hal, wherein Hal represents Cl, F, I or Br and R² is as described hereinabove for formula II in the presence of a base optionally in the presence of a solvent.
